(19) United States
(12) Patent Application Publication
(10) Pub. No.: US 2002/0192748 A1
(43) Pub. Date: Dec. 19, 2002

(54) Novel polynucleotides and polypeptides encoded thereby

(76) Inventors: Luca Rastelli, Guilford, CT (US); Richard A. Shimkets, West Haven, CT (US); Bryan Zerhusen, Branford, CT (US); Uriel M. Malyankar, Branford, CT (US); Muralidhara Padigaru, Bronx, NY (US)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 09/908,193

(22) Filed: Jul. 18, 2001

Related U.S. Application Data
(60) Provisional application No. 60/218,901, filed on Jul. 18, 2000.
(60) Provisional application No. 60/218,870, filed on Jul. 18, 2000.
(60) Provisional application No. 60/218,875, filed on Jul. 18, 2000.
(60) Provisional application No. 60/220,912, filed on Jul. 26, 2000.
(60) Provisional application No. 60/220,273, filed on Jul. 24, 2000.
(60) Provisional application No. 60/221,233, filed on Jul. 27, 2000.
(60) Provisional application No. 60/221,650, filed on Jul. 28, 2000.

(51) Int. Cl. C12Q 1/68; C07H 21/04; C12P 21/02; C12N 5/06; C07K 14/435

(52) U.S. Cl. 435/69.1; 435/325; 435/320.1; 435/6; 530/350; 536/23.2

(57) ABSTRACT

Disclosed herein are nucleic acid sequences that encode NOPE, cadherin, interferon-alpha-13, ADAM, ankyrin repeat-containing, transpanin, and semaphorin related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. Ser. No. 60/220,273, filed Jul. 24, 2000; U.S. Ser. No. 60/221,650, filed Jul. 28, 2000; U.S. Ser. No. 60/221,233, filed Jul. 27, 2000; U.S. Ser. No. 60/220,912, filed Jul. 26, 2000; U.S. Ser. No. 60/218,875, filed Jul. 18, 2000, U.S. Ser. No. 60/218,870, filed Jul. 18, 2000; and U.S. Ser. No. 60/218,901, filed Jul. 18, 2000 each of which is incorporated herein by reference in its entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4a, NOV4b, NOV5a, NOV5b, NOV6, and NOV7 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, and 17. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, and 18. The nucleic acid can be, for example, a genomic DNA fragment or a cDNA molecule that includes the nucleic acid sequence of any of SEQ ID NOS: 1, 3, 5, 7, 9, 11, 13, 15, and 17.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, and 17) or a complement of said oligonucleotide.

Also included in the invention are substantially purified NOVX polypeptides (SEQ ID NOS: 2, 4, 6, 8, 10, 12, 14, 16, and 18). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., developmental disorders, endocrine disorders, vascular disorders, infectious disease, anorexia, cancer, neurodegenerative disorders, lung disorders, reproductive disorders, Alzheimer's Disease, Parkinson's disease, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: endocrine disorders; developmental disorders; gastrointestinal diseases; lung diseases; respiratory disorders; vascular diseases; blood disorders; autoimmune and immune disorders; multiple sclerosis; inflammatory disorders and Hepatitis C; Trauma; regeneration (in vitro and in vivo); viral/bacterial/parasitic infections; hyperthyroidism; hypothyroidism; endometriosis; fertility; angiogenesis; hypertension; stroke; ischemia; arteriosclerosis; aneurysms; stroke; and bleeding disorders; Bare lymphocytic syndrome; type II; hereditary spherocytosis; elliptocytosis; pyropoikilocytosis; hemolytic anemia; Werner syndrome (scleroderma-like skin changes); juvenile rheumatoid arthritis; Graves disease; wound healing; X-linked mental retardation; and fertility disorders; psychotic and neurological disorders; neuronal degeneration; including but not limited to Parkinson's and Alzheimer's disease; dysplastic nevi and cancer; including but not limited to; glioma; leukemia; melanoma; pancreatic adenocarcinoma; non-Hodgkin's lymphoma; renal cancer; hepatocellular carcinomas; and myeloid leukemia lung or breast cancer.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from endocrine disorders; developmental disorders; gastrointestinal diseases; lung diseases; respiratory disorders; vascular diseases; blood disorders; autoimmune and immune disorders; multiple sclerosis; inflammatory disorders and Hepatitis C; Trauma; regeneration (in vitro and in vivo); viral/bacterial/parasitic infections; hyperthyroidism; hypothyroidism; endometriosis; fertility; angiogenesis; hypertension; stroke; ischemia; arteriosclerosis; aneurysms; stroke; and bleeding disorders; Bare lymphocytic syndrome; type II; hereditary spherocytosis; elliptocytosis; pyropoikilocytosis; hemolytic anemia; Werner syndrome (scleroderma-like skin changes); juvenile rheumatoid arthritis; Graves disease; wound healing; X-linked mental retardation; and fertility disorders; psychotic and neurological disorders; neuronal degeneration; including but not limited to Parkinson's and Alzheimer's disease; dysplastic nevi and cancer; including but not limited to; glioma; leukemia; melanoma; pancreatic adenocarcinoma; non-Hodgkin's lymphoma; renal cancer; hepatocellular carcinomas; and myeloid leukemia lung or breast cancer.

The invention further includes a method for screening for a modulator of disorders or syndromes including, e.g., developmental disorders, endocrine disorders, vascular disorders, infectious disease, anorexia, cancer, neurodegenerative disorders, lung disorders, reproductive disorders, Alzheimer's Disease, Parkinson's disease, immune and autoimmune disorders, and hematopoietic disorders, or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., developmental disorders, endocrine disorders, vascular disorders, infectious disease, anorexia, cancer, neurodegenerative disorders, lung disorders, reproductive disorders, Alzheimer's Disease, Parkinson's disease, immune and autoimmune disorders, and hematopoietic disorders, or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., developmental disorders, endocrine disorders, vascular disorders, infectious disease, anorexia, cancer, neurodegenerative disorders, lung disorders, reproductive disorders, Alzheimer's Disease, Parkinson's disease, immune and autoimmune disorders, and hematopoietic disorders, or other disorders related to cell signal processing and metabolic pathway modulation. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., developmental disorders, endocrine disorders, vascular disorders, infectious disease, anorexia, cancer, neurodegenerative disorders, lung disorders, reproductive disorders, Alzheimer's Disease, Parkinson's disease, immune and autoimmune disorders, and hematopoietic disorders, or other disorders related to cell signal processing and metabolic pathway modulation.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1 is homologous to members of the NOPE/PUNC immunoglobulin superfamily of cell surface proteins. NOPE protein is expressed during embryonic development in the notochord, in the developing skeletal muscles, and later in the ventricular zone of the nervous system. Thus, the NOVI nucleic acids, polypeptides, antibodies and related compounds according to the invention will be useful in therapeutic and diagnostic applications in disorders characterized by cell signaling, cell migration, invasion and tumor metastasis, e.g., nervous system dysfunction.

NOV2 is homologous to the Cadherin superfamily of cell adhesion proteins. Thus NOV2 may function similarly to other members of the Cadherin protein superfamily. Consequently, the NOV2 nucleic acids, polypeptides, antibodies and related compounds according to the invention will be useful in treating a variety of conditions, including, e.g., immune deficiencies and disorders, viral, bacterial and other infections, and cell proliferative disorders.

NOV3 is homologous to the Interferon superfamily of cytokines that includes interferon-alpha 13. Recombinant alpha interferons are approved worldwide for the treatment of a variety of cancers and diseases of virologic origin. Thus, the NOV3 nucleic acids and polypeptides, antibodies and related compounds according to the invention will be useful in therapeutic applications in various disorders involving interferon-alpha 13 and/or other members of the same family. The use of interferon in the treatment of non-Hodgkin's lymphomas is a specific example.

NOV4 is homologous to members of the ADAM transmembrane protein superfamily of cell surface proteins. The ADAM proteins contain both disintegrin and metalloprotease domains and, thus, these proteins potentially have both cell adhesion and protease activity. Accordingly, NOV4 nucleic acids, polypeptides, antibodies and related compounds according to the invention will be useful, for example, in therapeutic and diagnostic applications in various immune, developmental, and reproductive disorders.

NOV5 is homologous to the ankyrin repeat-containing family of proteins. Thus NOV5 nucleic acids, polypeptides, antibodies and related compounds according to the invention will be useful in treating a variety of conditions, including, e.g., immune deficiencies and disorders, viral, bacterial and other infections, and cell proliferative disorders.

NOV6 is homologous to the Tetraspanin superfamily of proteins shown to stimulate or modulate cell growth and control cell adhesion and movement. Thus, the NOV6 nucleic acids, polypeptides, antibodies and related compounds according to the invention will be useful in therapeutic and diagnostic applications in disorders characterized by cell signaling, cell migration, invasion and tumor metastasis, e.g., glioma.

NOV7 is homologous to members of the Semaphorin superfamily of proteins involved in cell signaling. Specifically, semaphorin-like proteins have been shown to play a critical role in the guidance of growth cones during neuronal development. Thus, the NOV7 nucleic acids, polypeptides, antibodies and related compounds according to the invention will be useful in therapeutic and diagnostic applications in disorders characterized by cell signaling, cell migration, invasion and tumor metastasis, e.g., glioma.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

The disclosed novel NOV1 nucleic acid (SEQ ID NO: 1) of 3741 nucleotides (also referred to AC068507A) is shown in Table 1A. Genomic DNA (GBNEW, Acc. No.:AC068507 and AC011846) were identified as having regions of high homology to the mouse NOPE gene. These sequences were initially identified on chromosome 15 by TblastN using GenBank file run against the Genomic files made available from GenBank. The genomic clone AC068507 contains the full length gene for human NOPE protein. An ORF begins with an ATG initiation codon at nucleotides 1-3 and ends with a TCT codon at nucleotides 3739-41.

TABLE 1A.
NOV1 Nucleotide Sequence (SEQ ID NO:1)

ATGGCGCGGGGGGACGCCGGCCGCGGCCGCGGGCTCCTCGCGTTGACCTTCTGCCTGTTGGCCGCGC
GCGGGGAGCTGCTGTTGCCCCAGGAGACGACTGTGGAGCTGAGCTGTGGAGTGGGGCCACTGCAAGT
GATCCTGGGCCCAGAGCAGGCTGCAGTGCTAAACTGTAGCCTGGGGGCTGCTGCCGCTGGACCCCCC
ACCAGGGTGACCTGGAGCAAGGATGGGGACACCCTGCTGGAGCACGACCACTTACACCTGCTGGCCA
ATGGTTCCCTGTGGCTGTCCCAGCCACTAGCACCCAATGGCAGTGACGAGTCAGTCCCTGAGGCTGT
GGGGGTCATTGAAGGCAACTATTCGTGCCTAGCCCACGGNCCCCCTCGAGTGCTGGCCAGCCAGACT
GCTGTCGTCAAGCTTGCCAGTCTCGCAGACTTCTCTCTGCACCCGGAGTCTCAGACGGTGGAGGAGA
ACGGGACAGCTCGCTTTGAGTGCCACATTGAAGGGCTGCCAGCTCCCATCATTACTTGGGAGAAGGA
CCAGGTGACATTGCCTGAGGAGCCTCGGCTCATCGTGCTTCCCAACGGCGTCCTTCAGATCCTGGAT
GTTCAGGAGAGTGATGCAGGCCCCTACCGCTGCGTGGCCACCAACTCAGCTCGCCAGCACTTCAGCC
AGGAGGCCCTACTCAGTGTGGCCCACAGAGGTTCCCTGGCGTCCACCAGGGGGCAGGACGTGGTCAT
TGTGGCAGCCCCAGAGAACACCACAGTGGTGTCTGGCCAGAGTGTGGTGATGGAATGTGTGGCCTCA
GCTGACCCCACCCCTTTTGTGTCCTGGGTCCGAGACGGGAAGCCCATCTCCACAGATGTCATCGTCC
TGGGCCGCACCAACCTACTAATTGCCAACGCGCAGCCCTGGCACTCCGGCGTCTATGTCTGCCGCGC
CAACAAGCCCCGCACGCGCGACTTCGCCACTGCAGCCGCTGAGCTCCGTGTGCTGCTAGCGGCTCCC
GCCATCACTCAGGCGCCCGAGGCGCTGTCGCGGACGCGGGCGAGCACAGCGCGCTTCGTGTGCCGCG
CGTCGGGGGAGCCGCGGCCAGCGCTGCGCTGGCTGCACAACGGGGCGCCGCTGCGGCCCAACGGGCG
CGTCAAGGTCCAGGGCGGCGGTGGCAGCCTGGTCATCACACAGATCGGCCTGCAGGACGCCGGCTAC
TACCAGTGCGTGGCTGAGAACAGCGCGGGAATGGCGTGCGCTGCCGCGTCGCTGGCCGTGGTGGTGC
GCGAGGGGCTGCCCAGCGCCCCCACGCGGGTCACTGCTACGCCACTGAGCAGCTCCGCTGTGTTGGT
GGCCTGGGAGCGGCCCGAGATGCACAGCGAGCAGATCATCGGCTTCTCTCTCCACTACCAGAAGGCA
CGGGGTATGGACAATGTGGAATACCAGTTTGCAGTGAACAACGACACCACAGAACTACAGGTTCGGG
ACCTGGAACCCAACACAGATTATGAGTTCTACGTGGTGGCCTACTCCCAGCTGGGAGCCAGCCGCAC
CTCCACCCCAGCACTGGTGCACACACTGGATGATGTCCCCAGTGCAGCACCCCAGCTCTCCCTGTCC
AGCCCCAACCCTTCGGACATCAGGGTGGCGTGGCTGCCCCTGCCCCCCAGCCTGAGCAATGGGCAGG
TGGTGAAGTACAAGATAGAATACGGTTTGGGAAAGGAAGATCAGATTTTCTCTACTGAGGTGCGAGG
AAATGAGACACAGCTTATGCTGAACTCGCTTCAGCCAAACAAGGTGTATCGAGTACGGATTTCGGCT
GGTACAGCAGCCGGCTTCGGGGCCCCCTCCCAGTGGATGCATCACAGGACGCCCAGTATGCACAACC
AGAGCCATGTCCCTTTTGCCCCTGCAGAGTTGAAGGTGCAGGCAAAGATGGAGTCCCTGGTCGTGTC
ATGGCAGCCACCCCCTCACCCCACCCAGATCTCTGGCTACAAACTATATTGGCGGGAGGTGGGGGCT
GAGGAGGAGGCCAATGGCGATCGCCTGCCAGGGGGCCGTGGAGACCAGGCTTGGGATGTGGGGCCTG
TCCGGCTCAAGAAGAAAGTGAAGCAGTATGAGCTGACCCAGCTAGTCCCTGGCCGGCTGTACGAGGT
GAAGCTCGTGGCTTTCAACAAACATGAGGATGGCTATGCAGCAGTGTGGAAGGGCAAGACGGAGAAG
GCGCCGGCACCAGACATGCCTATCCAGAGGGGACCACCCCTGCCTCCAGCCCACGTCCATGCGGAAT
CAAACAGCTCCACATCCATCTGGCTTCGGTGGAAAAAGCCAGATTTCACCACAGTCAAGATTGTCAA
CTACACTGTGCGCTTCAGCCCCTGGGGGCTCAGGAATGCCTCCCTGGTCACCTATTACAGTTCTGGA
GAAGACATCCTCATTGGCGGCTTGAAGCCATTCACCAAATACGAGTTTGCAGTGCAGTCTCACGGCG
TGGACATGGATGGGCCTTTCGGCTCTGTGGTGGAGCGCTCCACCCTGCCTGACCGTCCCTCCACACC
CCCATCCGACCTGCGACTGAGCCCCCTGACACCGTCCACGGTTCGGCTGCACTGGTGCCCCCCCACA
GAGCCCAACGGGGAGATCGTGGAGTATCTGATCCTGTACAGCAGCAACCACACGCAGCCTGAGCACC
AGTGGACCTTGCTCACCACGCAGGGTGAGGGAAACATCTTCAGTGCTGAGGTCCATGGCCTGGAGAG
CGACACTCGGTACTTCTTCAAGATGGGGGCGCGCACAGAGGTGGGACCTGGGCCTTTCTCCCGCCTG
CAGGATGTGATCACGCTCCAGGAGAAGCTGTCAGACTCGCTGGACATGCACTCAGTCACGGGCATCA
TCGTGGGTGTCTGCCTGGGCCTCCTCTGCCTCCTGGCCTGCATGTGTGCTGGCCTGCGCCGCAGCCC
CCACAGGGAATCCCTCCCAGGCCTGTCCTCCACCGCCACCCCCGGGAATCCCGCGCTGTACTCCAGA
GCTCGGCTTGGCCCCCCCAGCCCCCCAGCTGCCCATGAATTGGAGTCCCTTGTGCACCCCCATCCCC
AGGACTGGTCCCCGCCACCCTCAGACGTGGAGGACAGGGCTGAAGTGCACAGCCTTATGGGTGGCGG
TGTTTCTGAAGGCCGGAGTCACTCCAAAAGAAAGGTAAGTGCTCAACCAAGCGGGCTGAGCTGGGCT
GGTTCCTGGGCAGGCTGTGAGCTGCCCCAGGCAGGCCCCCGGCCGGCTCTGACCCGGGCCCTGCTGC
CCCCTGCTGGAACTGGGCAGACGCTGTTGCTGCAGGTTCTCTGCTCTGATCAGGGCAATGGGAGGAA
GAAGTCACCCCCAGCCTGCAGGAACCAGGTGGAGGCTGAAGTCATTGTCCACTCTGACTTTAGTGCA
TCTAACGGGAACCCTGACCTCCATCTCCAAGACCTGGAGCCTGAGGACCCCCTGCCTCCAGAGGCTC
CTGATCTCATCTCGGGTGTTGGGGATCCAGGGCAGGGGGCAGCCTGGCTGGACAGGGAGTTGGGAGG
GTGTGAGCTGGCAGCCCCCGGGCCAGACAGACTTACCTGCTTGCCAGAGGCAGCCAGTGCTTCCTGC
TCCTACCCGGACCTCCAGCCAGGCGAGGTGCTAGAGGAGACCCCTGGAGATAGCTGCCAGCTCAAAT
CCCCCTGCCCTCTAGGAGCCAGCCCAGGCCTGCCCAGATCCCCGGTCTCCTCCTCT

The NOV1 protein (SEQ ID NO:2) encoded by SEQ ID NO:1 is 1247 amino acid residues in length, has a molecular weight of 133821.8 Daltons, and is presented using the one-letter amino acid code in Table 1B. The SignalP and Psort indicate that this sequence has a signal peptide and is likely to be a Type I membrane protein. The Psort profile for NOV1 predicts that these sequences have a signal peptide and are likely to be localized at the plasma membrane with a certainty of 0.4600. The Signal P predicts a likely cleavage site for a NOV1 peptide is between positions 24 and 25, i.e., at the dash in the sequence ARG-EL.

TABLE 1B.
Encoded NOV1 protein sequence (SEQ ID NO:2)

MARGDAGRGRGLLALTFCLLAARGELLLPQETTVELSCGVGPLQVILGPEQAAVLNCSLGAA
AAGPPTRVTWSKDGDTLLEHDHLHLLANGSLWLSQPLAPNGSDESVPEAVGVIEGNYSCLAHGPP
RVLASQTAVVKLASLADFSLHPESQTVEENGTARFECHIEGLPAPIITWEKDQVTLPEEPRLIVL
PNGVLQILDVQESDAGPYRCVATNSARQHFSQEALLSVAHRGSLASTRGQDVVIVAAPENTTVVS
GQSVVMECVASADPTPFVSWVRDGKPISTDVIVLGRTNLLIANAQPWHSGVYVCRANKPRTRDFA
TAAAELRVLLAAPAITQAPEALSRTRASTARFVCRASGEPRPALRWLHNGAPLRPNGRVKVQGGG
GSLVITQIGLQDAGYYQCVAENSAGMACAAASLAVVVREGLPSAPTRVTATPLSSSAVLVAWERP
EMHSEQIIGFSLHYQKARGMDNVEYQFAVNNDTTELQVRDLEPNTDYEFYVVAYSQLGASRTSTP
ALVHTLDDVPSAAPQLSLSSPNPSDIRVAWLPLPPSLSNGQVVKYKIEYGLGKEDQIFSTEVRGN
ETQLMLNSLQPNKVYRVRISAGTAAGFGAPSQWMHHRTPSMHNQSHVPFAPAELKVQAKMESLVV
SWQPPPHPTQISGYKLYWREVGAEEEANGDRLPGGRGDQAWDVGPVRLKKKVKQYELTQLVPGRL
YEVKLVAFNKHEDGYAAVWKGKTEKAPAPDMPIQRGPPLPPAHVHAESNSSTSIWLRWKKPDFTT
VKIVNYTVRFSPWGLRNASLVTYYSSGEDILIGGLKPFTKYEFAVQSHGVDMDGPFGSVVERSTL
PDRPSTPPSDLRLSPLTPSTVRLHWCPPTEPNGEIVEYLILYSSNHTQPEHQWTLLTTQGEGNIF
SAEVHGLESDTRYFFKMGARTEVGPGPFSRLQDVITLQEKLSDSLDMHSVTGIIVGVCLGLLCLL
ACMCAGLRRSPHRESLPGLSSTATPGNPALYSRARLGPPSPPAAHELESLVHPHPQDWSPPPSDV
EDRAEVHSLMGGGVSEGRSHSKRKVSAQPSGLSWAGSWAGCELPQAGPRPALTRALLPPAGTGQT
LLLQVLCSDQGNGRKKSPPACRNQVEAEVIVHSDFSASNGNPDLHLQDLEPEDPLPPEAPDLISG
VGDPGQGAAWLDRELGGCELAAPGPDRLTCLPEAASASCSYPDLQPGEVLEETPGDSCQLKSPCP
LGASPGLPRSPVSSS

A search against the Patp database, a proprietary database that contains sequences published in patents and patent publications, yielded several homologous proteins shown in Table 1C.

TABLE 1C
Patp Results for NOV1

| | | | Smallest |
|---|---|---|---|
| | | | Sum |
| Sequences producing | Reading | High | Prob |
| High-scoring Segment Pairs: | Frame | Score | P(N) |
| >patp: AAR68553 Deleted in colorectal | +1 | 630 | 8e−72 |
| carcinoma (DCC) | | | |
| >patp: AAY33498 Human DCC protein | +1 | 1147 | 8e−72 |
| >patp: AAB50693 Human UNC-40 protein | +1 | 1447 | 8e−72 |
| >patp: AAR13144 Deleted in Colorectal | +1 | 1728 | 2.8e−71 |
| Carcinomas | | | |

In a BLAST search of public sequence databases, it was found, for example, that the nucleic acid sequence has 2865 of 3350 (85%), identical to a Mus musculus NOPE mRNA (GENBANK-ID: AF 176694). The full amino acid sequence of the protein of the invention was found to have 1083 of 1249 (87%) amino acid residues identical to, and 1137 of 1249 (91%) residues positive with, the 1252 amino acid residue protein from Mus musculus (ptnr:SPTREMBL-ACC:AAF65930).

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the IIT BLAST analysis, matched the Query IIT sequence purely by chance is the E value. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences. Blasting is performed against public nucleotide databases such as GenBank databases and the GeneSeq patent database. For example, BLASTX searching is performed against public protein databases, which include GenBank databases, SwissProt, PDB and PIR.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

NOV1 also has homology to the proteins shown in the BLASTP data in Table 1D.

TABLE 1D
BLAST results for NOV1

| Gene Index/ | | Length | Identity | Positives | |
|---|---|---|---|---|---|
| Identifier | Protein/Organism | (aa) | (%) | (%) | Expect |
| ptnr:SPTREMBL- | Mus musculus | 1252 | 1089/1249 | 1141/1249 | 0.0 |
| ACC:Q9EQS9 DDM36 | protein | | (87%) | (91%) | |
| ptnr:SPTREMBL- | Mus musculus | 1253 | 1089/1250 | 1141/1250 | 0.0 |
| ACC:Q9EQS8 DDM36E | protein | | (87%) | (91%) | |
| ptnr:SPTREMBL- | NEIGHBOR OF PUNC | 1252 | 1083/1249 | 1137/1249 | 0.0 |
| ACC:Q9JLI1 | Ell PROTEIN | | (86%) | (91%) | |
| [Mus musculus] | | | | | |
| ptnr:SPTREMBL- | KIAA1G28 PROTEIN | 980 | 918/961 | 927/961 | 0.0 |
| ACC:Q9HCE4 | [Homo sapiens] | | (95%) | (96%) | |
| ptnr:SPTREMBL- | PUTATIVE NEURONAL | 793 | 235/578 | 331/578 | 1.le- |
| ACC:O70246 | CELL ADHESION | | (40%) | (57%) | 100 |
| | MOLECULE (PUNC) | | | | |
| | (PUTATIVE NEURONAL | | | | |
| | CELL ADHESION | | | | |
| | MOLECULE, SHORT | | | | |
| | FORM) | | | | |
| | [Mus musculus] | | | | |

A multiple sequence alignment is given in Table 1E, with the NOV1 protein being shown on line 1 in Table 1E in a ClustalW analysis, and comparing the NOVI protein with the related protein sequences shown in Table ID. This BLASTP data is displayed graphically in the ClustalW in Table 1E.

TABLE 1E
ClustalW Analysis of NOV1

1) >NOV1; SEQ ID NO:1
2) >Q9EQS9/[Mus muscuulus]; SEQ ID NO:19
3) >Q9EQS8/[Mus musculus]; SEQ ID NO:20
4) >Q9JLI1/Neighbor of PUNC E11 protein [Mus musculus]; SEQ ID NO:21
5) >Q9HCE4/KIAA1628 protein [Homo sapiens]; SEQ ID NO:22
6) >O70246/Putative neuronal adhesion moleculae (PUNC)m short form [Mus musculus]; SEQ ID NO:23

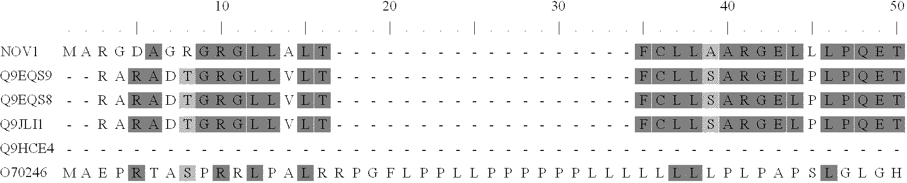

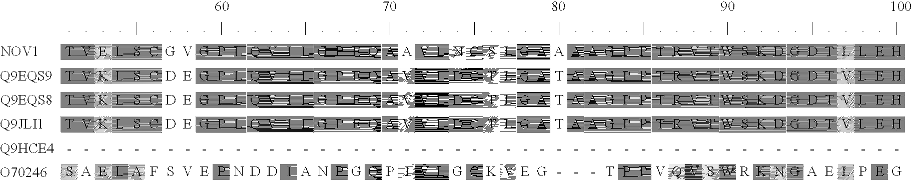

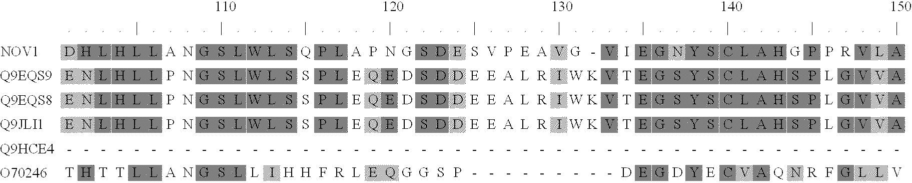

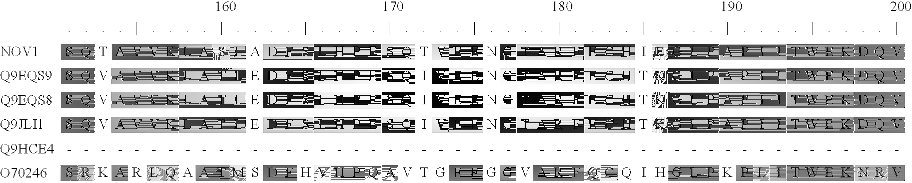

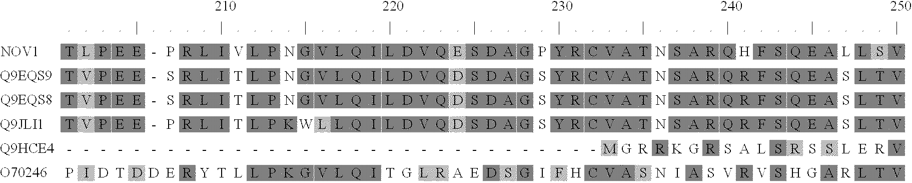

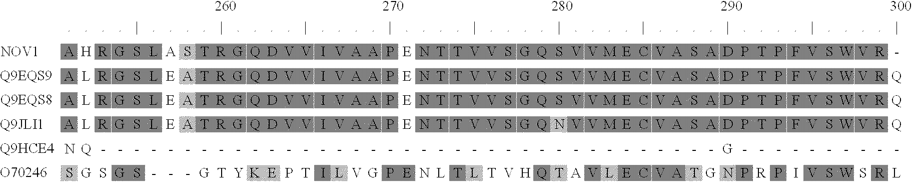

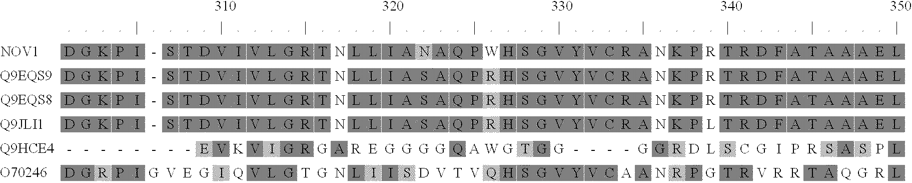

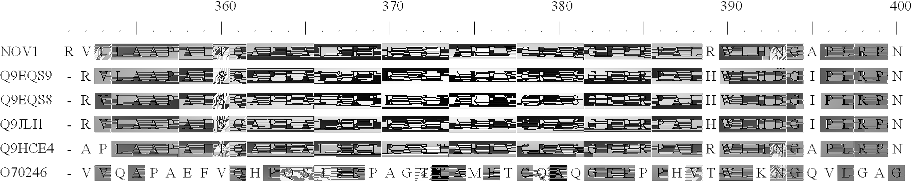

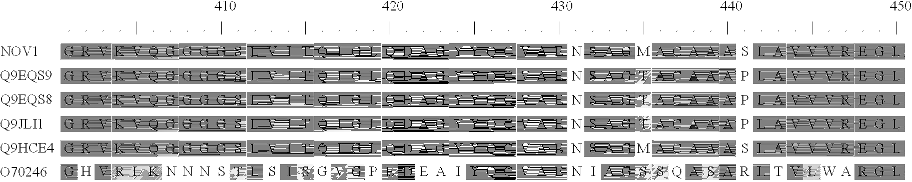

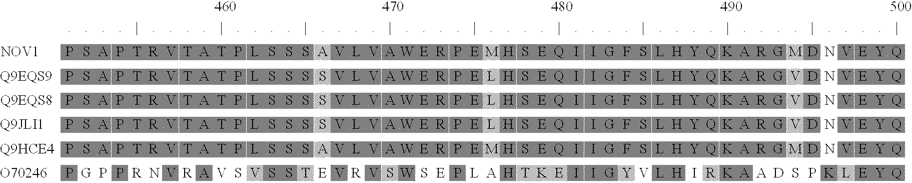

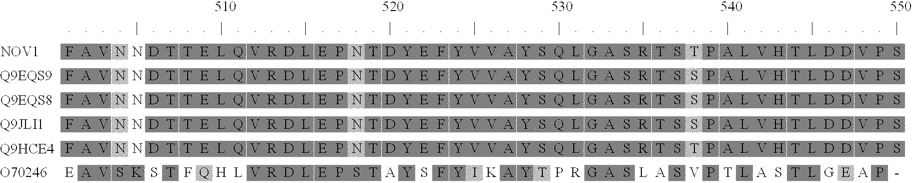

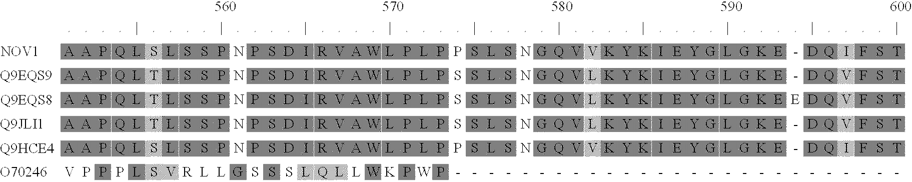

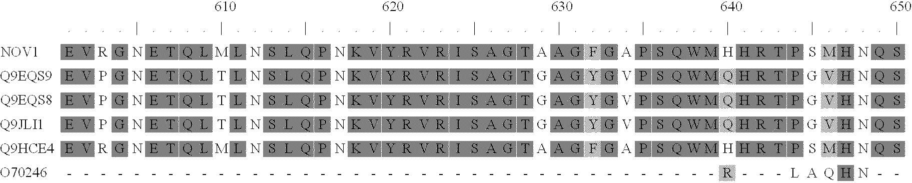

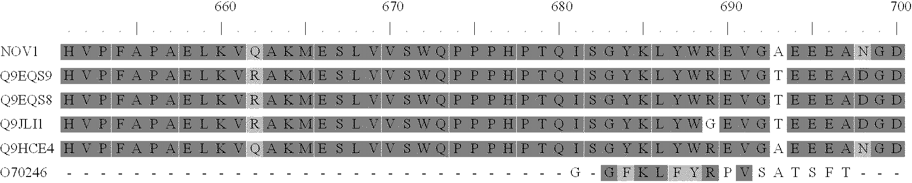

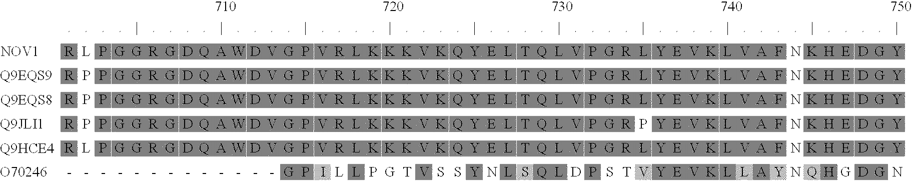

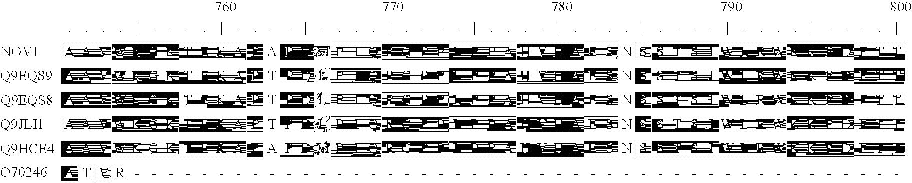

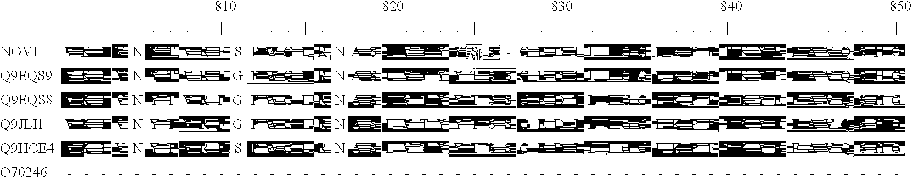

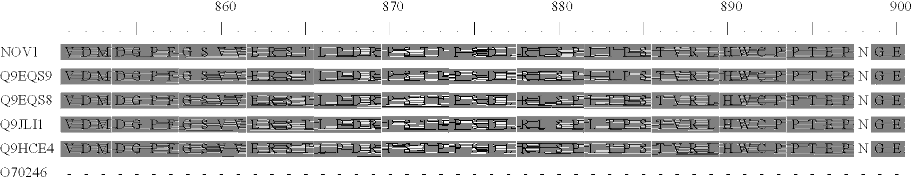

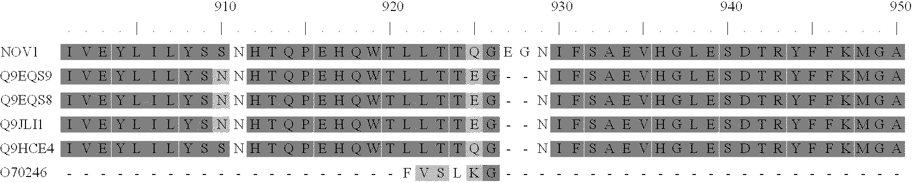

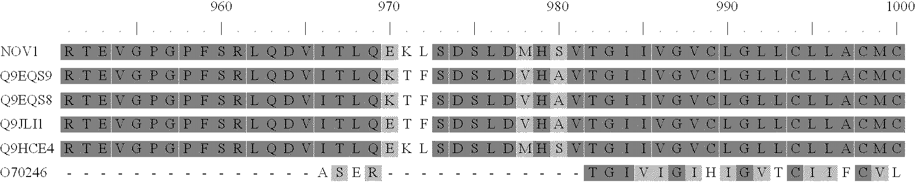

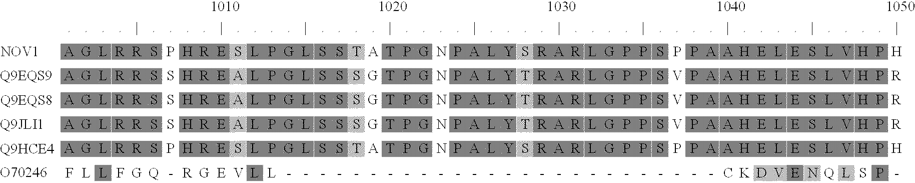

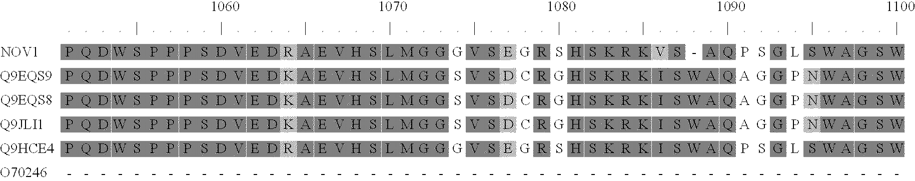

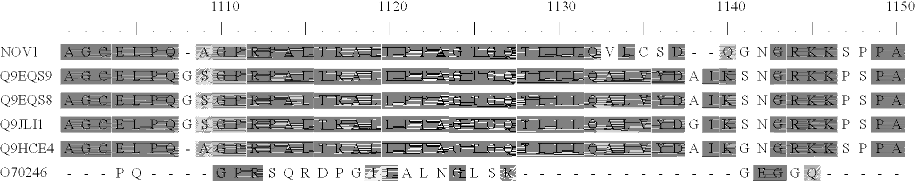

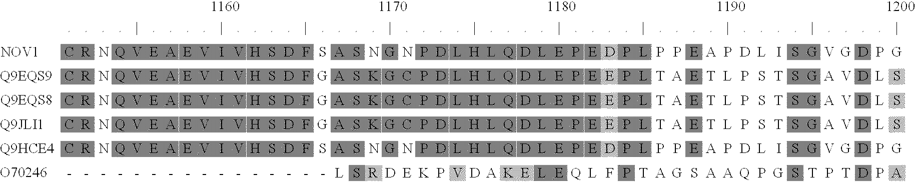

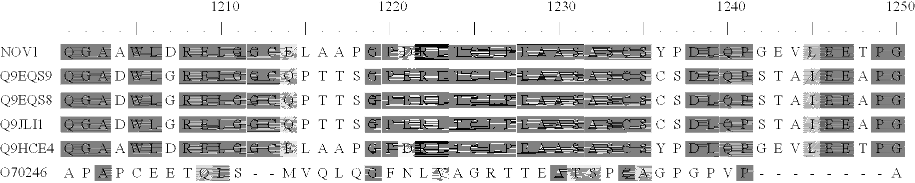

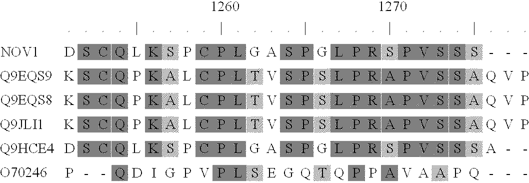

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 1F lists the domain description from DOMAIN analysis results against NOV1.

TABLE 1F
Domain Analysis of NOV1

| | Region of | | |
|---|---|---|---|
| Model | Homology | Score (bits) | E value |
| Immunoglobulin | 50-123 | 10.5 | 0.11 |
| Immunoglobulin | 157-214 | 35 | 2.6e−09 |
| Immunoglobulin | 258-313 | 39.5 | 1.1e−10 |
| Immunoglobulin | 349-407 | 30.9 | 5.2e−08 |
| Fibronectin type III | 429-515 | 84 | 3.1e−21 |
| Fibronectin type III | 527-613 | 54.5 | 2.3e−12 |
| Fibronectin type III | 630-729 | 42 | 1.4e−08 |
| Fibronectin type III | 750-934 | 39.3 | 8.9e−08 |
| Fibronectin type III | 846-936 | 60.8 | 2.9e−14 |

The presence of protein regions in NOV1 that are homologous to an immunoglobulin domain and a fibronectin type III (IPR003961) domain is consistent with the identification of NOV 1 protein as a NOPE/PUNC-like protein. This indicates that the NOV 1 sequence has properties similar to those of other proteins known to contain these domains.

The domain and protein similarity information for the invention suggests that this gene may function as “NOPE/PUNC.” As such, the NOV1 protein of the invention may function as a cell adhesion receptor, specifically a receptor tyrosine kinase class V, in the tissues of expression. NOV1 is implicated, therefore, in disorders involving these tissues, such as, for example, retinitis pigmentosa, polydactyly, obesity, hypogenitalism, mental retardation, and renal cancer.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

The novel nucleic acid encoding the NOV 1 of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOVI protein has multiple hydrophilic regions, each of which can be used as an immunogen. The hydropathy plot for invention shows that the protein sequence has an amino terminal hydrophobic region, which could function as a signal peptide to target this sequence to the plasma membrane.

NOV2

The disclosed novel NOV2 nucleic acid (SEQ ID NO:3) of 1857 nucleotides (also referred to SC101760703_A) is shown in Table 2A. In a search of CuraGen's proprietary human expressed sequence assembly database, s3aq: 101760703 (577 nucleotides) was identified as having 95% homology to this predicted gene sequence. An ORF begins with an ATG initiation codon at nucleotides 28-30 and ends with a TGA codon at nucleotides 1834-1836. A putative untranslated region and/or downstream from the termination codon is underlined in Table 2A, and the start and stop codons are in bold letters.

TABLE 2A.
NOV2 Nucleotide Sequence (SEQ ID NO:3)

CTGGGCATTGTGCCTTCCTGCCCTGGCATGAAGAGCCCCAGGCCCCACCTCCTGCTACCATTGCTGC
TGCTGCTGCTGCTGCTGCTGGGGGCTGGGGTGCCAGGTGCCTGGGGTCAGGCTGGGAGCCTGGACTT
GCAGATTGATGAGGAGCAGCCAGCGGGTACACTGATTGGCGACATCAGTGCGGGGCTTCCGGCAGGC
ACGGCAGCTCCTCTCATGTACTTCATCTCTGCCCAAGAGGGCAGCGGCGTGGGCACAGACCTGGCCA
TTGACGAACACAGTGGGGTCGTCCGTACAGCCCGTGTCTTGGACCGTGAGCAGCGGGACCGCTACCG
CTTCACTGCAGTCACTCCTGATGGTGCCACCGTAGAAGTTACAGTGCGAGTGGCTGACATCAACGAC
CATGCTCCAGCCTTCCCACAGGCTCGGGCTGCCCTGCAGGTACCTGAGCATACAGCTTTTGGCACCC
GCTACCCACTGGAGCCTGCTCGTGATGCAGATGCTGGGCGTCTGGGAACCCAGGGCTATGCGCTATC
TGGTGATGGGGCTGGAGAGACCTTCCGGCTGGAGACACGCCCCGGTCCAGATGGGACTCCAGTACCT
GAGCTGGTAGTTACTGGGGAACTGGACCGAGAGAACCGCTCACACTATATGCTACAGCTGGAGGCCT
ATGATGGTGGTTCACCCCCCCGGAGGGCCCAGGCCCTGCTGGACGTGACACTGCTGGACATCAATGA
CCATGCCCCGGCTTTCAATCAGAGCCGCTACCATGCTGTGGTGTCTGAGAGCCTGGCCCCTGGCAGT
CCTGTCTTGCAGGTGTTCGCATCTGATGCCGATGCTGGTGTCAATGGGGCTGTGACTTACGAGATCA
ACCGGAGGCAGAGCGAGGGTGATGGACCCTTCTCCATCGACGCACACACGGGGCTGCTGCAGTTAGA
GCGGCCACTGGACTTTGAGCAGCGGCGGGTCCATGAACTGGTGGTGCAAGCACGAGATGGTGGGGCT
CACCCTGAGCTGGGCTCGGCCTTTGTGACTGTGCATGTGCGAGATGCCAATGACAATCAGCCCTCCA
TGACTGTCATCTTTCTCAGTGCAGATGGCTCCCCCCAAGTGTCTGAGGCCGCCCCACCTGGACAGCT
CGTTGCTCGCATCTCTGTGTCAGACCCAGATGATGGTGACTTTGCCCATGTCAATGTGTCCCTGGAA
GGTGGAGAGGGCCACTTTGCCCTAAGCACCCAAGACAGCGTCATCTATCTGGTGTGTGTGGCTCGGC
GGCTGGATCGAGAGGAGAGGGATGCCTATAACTTGAGGGTTACAGCCACACACTCAGGCTCACCTCC
ACTGCGGGCTGAGGCTGCCTTTGTGCTGCACGTCACTGATGTCAACGACAATGCACCTGCCTTTGAC
CGCCAGCTCTACCGACCTGAGCCCCTGCCTGAGGTTGCGCTGCCTGGCAGCTTTGTAGTGCGGGTGA
CTGCTCGGGATCCTGACCAAGGCACCAATGGTCAGGTCACTTATAGCCTAGCCCCTGGCGCCCACAC
CCACTGGTTCTCCATTGACCCCACCTCAGGCATTATCACTACGGCTGCCTCACTGGACTATGAGTTG
GAACCTCAGCCACAGCTGATTGTGGTGGCCACAGATGGTGGCCTGCCCCCTCTAGCCTCCTCTGCCA
CAGTTAGCGTGGCCCTGCAAGATGTGAATGATAATGAGCCCCAATTCCAGAGGACTTTCTACAATGC
CTCACTGCCTGAGGGCACCCAGCCTGGAACTTGCTTCCTGCAGGTGGGACCAATGGGCTATGGCTTC
CAGACTTCTCTCCTCACTAGTGCCTGAAGCAAGAGGGCAACTCTCCAG

The NOV2 protein (SEQ ID NO:4) encoded by SEQ ID NO:3 is 602 amino acid residues in length, has a molecular weight of 64138.5 Daltons, and is presented using the one-letter amino acid code in Table 2B. Psort analysis predicts the protein of the invention to be localized outside the cell with a certainty of 0.8200. The Signal P predicts a likely cleavage site for a NOV2 peptide is between positions 29 and 30, i.e., at the dash in the sequence AWG-QA.

TABLE 2B.
Encoded NOV2 protein sequence (SEQ ID NO:4)

MKSPRPHLLLPLLLLLLLLLGAGVPGAWGQAGSLDLQIDEEQPAGTLIGDISAGLPAGTAAPLMY
FISAQEGSGVGTDLAIDEHSGVVRTARVLDREQRDRYRFTAVTPDGATVEVTVRVADINDHAPAF
PQARAALQVPEHTAFGTRYPLEPARDADAGRLGTQGYALSGDGAGETFRLETRPGPDGTPVPELV
VTGELDRENRSHYMLQLEAYDGGSPPRRAQALLDVTLLDINDHAPAFNQSRYHAVVSESLAPGSP
VLQVFASDADAGVNGAVTYEINRRQSEGDGPFSIDAHTGLLQLERPLDFEQRRVHELVVQARDGG
AHPELGSAFVTVHVRDANDNQPSMTVIFLSADGSPQVSEAAPPGQLVARISVSDPDDGDFAHVNV
SLEGGEGHFALSTQDSVIYLVCVARRLDREERDAYNLRVTATDSGSPPLRAEAAFVLHVTDVNDN
APAFDRQLYRPEPLPEVALPGSFVVRVTARDPDQGTNGQVTYSLAPGAHTHWFSIDPTSGIITTA
ASLDYELEPQPQLIVVATDGGLPPLASSATVSVALQDVNDNEPQFQRTFYNASLPEGTQPGTCFL
QVGPMGYGFQTSLLTSA

A search against the Patp database, a proprietary database that contains sequences published in patents and patent publications, yielded several homologous proteins shown in Table 2C.

TABLE 2C
Patp results for NOV2

| | | | |
|---|---|---|---|
| | | | Smallest |
| | | | Sum |
| Sequences producing | Reading | High | Prob |
| High-scoring Segment Pairs: | Frame | Score | P(N) |

| | | | |
|---|---|---|---|
| >patp: AAB95684 Human protein sequence | +1 | 812 | 1.9e−82 |
| SEQ ID NO: 18485 | | | |
| >patp: AAY41750 Human PRO731 protein | +1 | 810 | 3.1e−82 |
| sequence | | | |
| >patp: AAB44306 Human PRO731 | +1 | 810 | 3.1e−82 |
| (UNQ395) protein sequence | | | |

In a BLAST search of public sequence databases, it was found, for example, that the nucleic acid sequence has 968 of 1602 bases (60%) identical to a Drosophila melanogaster cadherin mRNA (GENBANK-ID: DRODACHSOU). The full amino acid sequence of the protein of the invention was found to have 276 of 576 amino acid residues (47%) identical to, and 361 of 576 residues (62%) positive with, the 3503 amino acid residue cadherin protein from Drosophila melanogaster (ptnr:SPTREMBL-ACC: Q24292). The global sequence homology (as defined by FASTA alignment with the full length sequence of this protein) is 54.762% amino acid homology and 47.789% amino acid identity.

NOV2 also has homology to the proteins shown in the BLASTP data in Table 2D.

TABLE 2D
BLAST results for NOV2

| Gene Index/ | | Length | Identity | Positives | |
|---|---|---|---|---|---|
| Identifier | Protein/Organism | (aa) | (%) | (%) | Expect |
| ptnr:SPTREMBL- | DACHSOUS PROTEIN | 3503 | 276/576 | 361/576 | 2.2e- |
| ACC:Q24292 | PRECURSOR | | (47%) | (62%) | 135 |
| | (ADHERIN) | | | | |
| | [Drosophila | | | | |
| | melanogaster] | | | | |
| ptnr:pir-id:IJFFTM | cadherin-related | 5147 | 205/550 | 287/550 | 1.0e- |
| | tumor suppressor | | (37%) | (52%) | 84 |
| | precursor | | | | |
| | [Drosophila | | | | |
| | melanogaster] | | | | |
| ptnr:SPTREMBL- | CDNA FLJ14078 FIS, | 1187 | 212/564 | 299/564 | 3.0e- |
| ACC:Q9H7Y6 | CLONE HEMBB1002044 | (37%) | (53%) | 82 | |
| | MODERATELY SIMILAR | | | | |
| | TO MUS MUSCULUS | | | | |
| | VASCULAR CADHERIN- | | | | |
| | 2 | | | | |
| | [Homo sapiens] | | | | |
| ptnr:SPTREMBL- | VASCULAR CADHERIN- | 1184 | 212/564 | 298/564 | 4.8e- |
| ACC:Q9NPG4 | 2 (PROTOCADHERIN | | (37%) | (52%) | 82 |
| 12) | (VASCULAR | | | | |
| ENDOTHELIAL | | | | | |
| CADHERIN 2) | | | | | |
| [Homo sapiens] | | | | | |
| ptnr:SPTREMBL- | PROTOCADHERIN2 | 932 | 222/581 | 317/581 | 8.5e- |
| ACC:O13129 | (PCDH2), COMPLETE | | (38%) | (54%) | 80 |
| | CDS | | | | |
| | [Gallus gallus] | | | | |

A multiple sequence alignment is given in Table 2E, with the NOV2 protein being shown on line 1 in Table 2E in a ClustalW analysis, and comparing the NOV2 protein with the related protein sequences shown in Table 2D. This BLASTP data is displayed graphically in the ClustalW in Table 2E.

TABLE 2E
ClustalW Analysis of NOV2

1) >NOV2; SEQ ID NO:3
2) >Q24292/Dachous protein precursor (adherin) [Drosophila melanogaster]; SEQ ID NO:24
3) >IJFFTM/Cahedrin-related tumor suppressor precurson [Drosophila melanogaster]; SEQ ID NO:25
4) >Q9H7Y6/cDNA similar to Mus musculus vascular cahedrin-2 [Homo sapiens]; SEQ ID NO:26
5) >Q9NPG4/Vascular cahedrin-2 (protocahedrin-2) [Homo sapiens]; SEQ ID NO:27
6) >O13129/Protocahedrin-2 (PCDH2) complete CDS [Gallus gallus]; SEQ ID NO:28

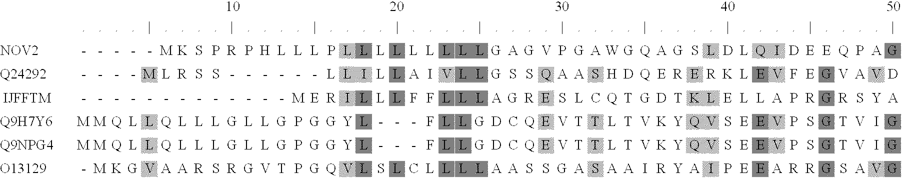

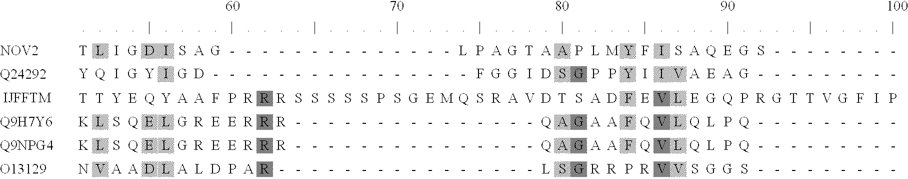

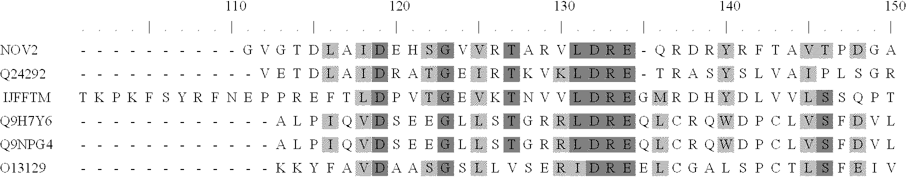

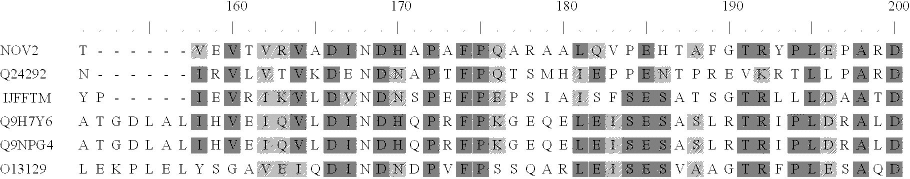

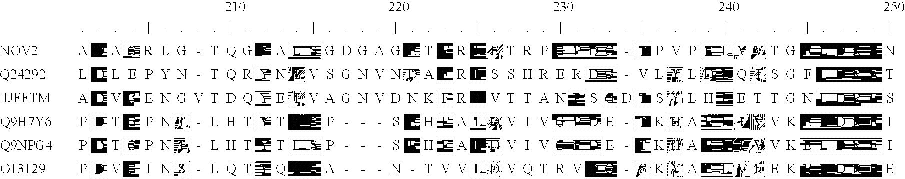

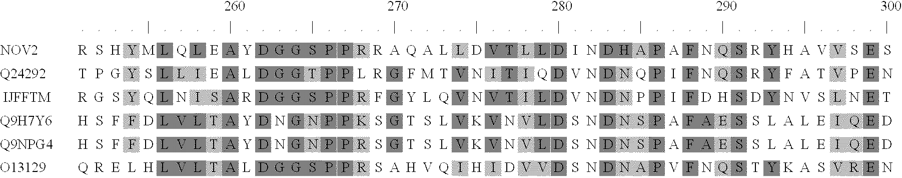

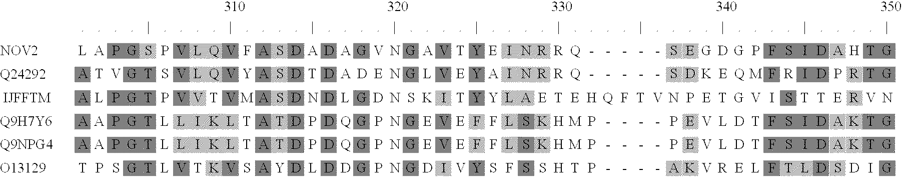

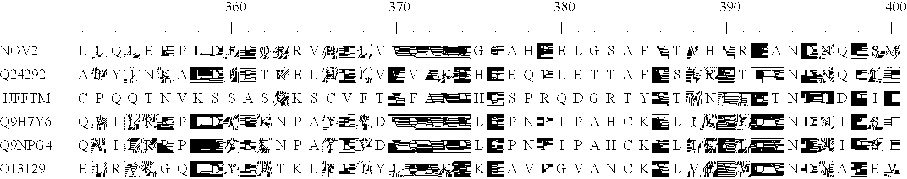

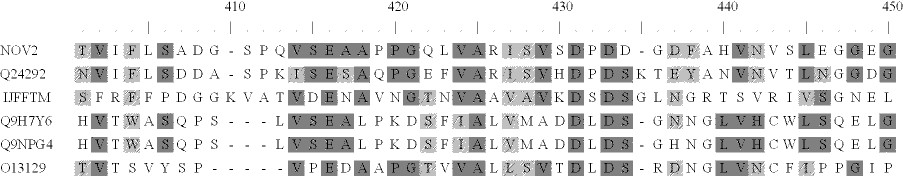

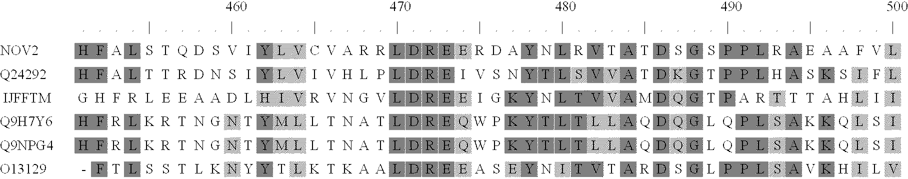

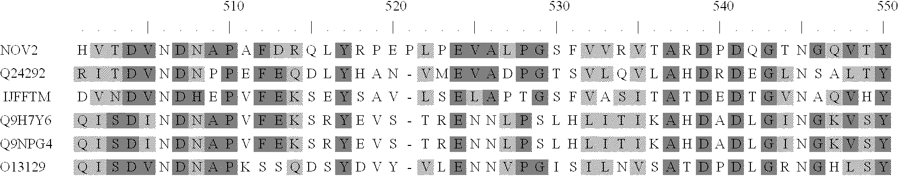

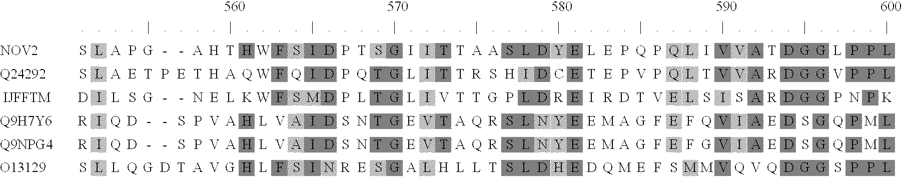

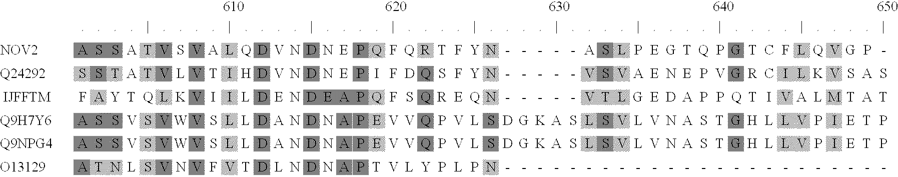

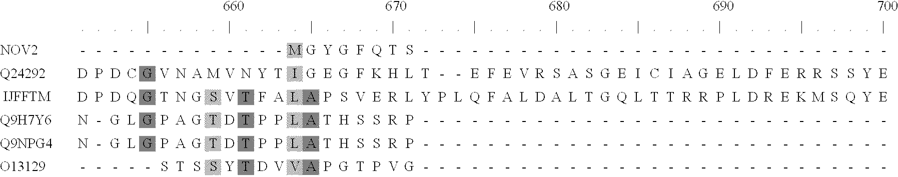

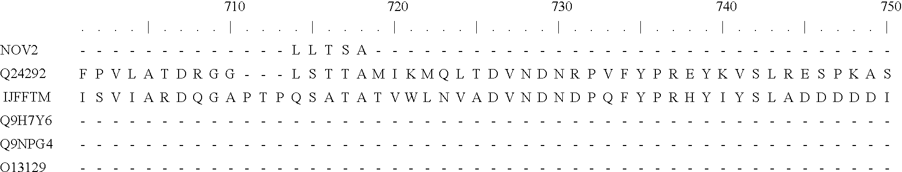

The NOV2 Clustal W alignment show in Table 2E was modified to end at amino residue 750. The data in Table 2E includes all of the regions overlapping with the 602 amino acid residues of the NOV2 protein sequence.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 2F lists the domain description from DOMAIN analysis results against NOV2. This indicates that the NOV2 sequence has properties similar to those of other proteins known to contain these domains.

TABLE 2F
Domain Analysis of NOV2

| | Region of | | |
|---|---|---|---|
| Model | Homology | Score (bits) | E value |
| Cadherin | 34-121 | 29.7 | 6.8e−05 |
| Cadherin | 135-233 | 44.2 | 2.9e−09 |
| Cadherin | 247-340 | 88.5 | 1.3e−22 |
| Cadherin | 358-450 | 59.9 | 5.6e−14 |
| Cadherin | 465-556 | 91.8 | 1.4e−23 |
| Gene66 | 542-560 | 5.1 | 1.7 |

The presence of protein regions in NOV2 that are homologous to cadherin domain (lPR002126) is consistent with the identification of NOV2 protein as a cadherin-like protein.

The above defined information for NOV2 suggests that this cadherin-like protein may function as a member of a “cadherin family.” Therefore, the NOV2 nucleic acids and proteins of the invention are useful in potential therapeutic applications implicated in various pathologies/disorders described and/or other pathologies/disorders. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

The novel nucleic acid encoding the cahedrin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen.

NOV3

The disclosed novel NOV3 nucleic acid (SEQ ID NO:5) of 632 nucleotides (also referred to GMba380p16_A) is shown in Table 3A. NOV3 encodes a novel interferon-alpha-13-like protein and was identified on chromosome 9 by TblastN using CuraGen Corporation's sequence file for interferon-alpha-13 precursor as run against the Genomic Daily Files made available by GenBank or from files downloaded from the individual sequencing centers. The nucleic acid sequence was predicted from the genomic Sequencing Center file ba380p16 by homology to a known interferon-alpha-13 precursor. An ORF begins with an ATG initiation codon at nucleotides 18-20 and ends with a TGA codon at nucleotides 618-620. A putative untranslated region upstream from the initiation codon and downstream from the termination codon is underlined in Table 3A, and the start and stop codons are in bold letters.

TABLE 3A.
NOV3 Nucleotide Sequence (SEQ ID NO:5)

e,uns CATCTGCAATATCTATGATGGCCTCGCCCTTTGCTTTACTGATGGCCCTGGTGGTGCTCAGCTGCAA
GTCAAGCTGCTCTCTGGGCTGTGATCTCCCTGAGACCCACAGCCTGGATAACAGGAGGACCTTGATG
CTCCTGGCACAAATGAGCAGAATCTCTCCTTCCTCCTGTCTGATGGACAGACATGACTTTGGATTTC
CCCAGGAGGAGTTTGATGGCAACCAGTTCCAGAAGGCTCCAGCCATCTCTGTCCTCCATGAGCTGAT
CCAGCAGATCTTCAACCTCTTTACCACAAAAGATTCATCTGCTGCTTGGGATGAGGACCTCCTAGAC
CAATTCTGCACCGAACTCTACCAGCAGCTGAATGACTTGGAAGCCTGTGTGATGCAGGAGGAGAGGG
TGGGAGAAACTCCCCTGATGAATGCGGACTCCATCTTGGCTGTGAAGAAATACTTCCGAAGAATCAC
TCTCTATCTGACAGAGAAGAAGTTAGGCCTGTGTGATTGGTGGGTTGCTAGAGCACCTATCCTGACA
ACCCTCTCTTTGTCCTGCAACTTGCAAGTAACTTTAGGTAGTAAGCTTTGCCTTCTGGTCCACCATG
TTACAATTGCTTTGTGACTCATAAAACAG

The NOV3 protein (SEQ ID NO:6) encoded by SEQ ID NO:5 is 200 amino acid residues in length, has a molecular weight of 22547.9 Daltons, and is presented using the one-letter amino acid code in Table 3B. The Psort profile for NOV3 predicts that this sequence is likely to be localized in the outside with a certainty of 0.5565. Using Signal P analysis, it is predicted that the protein of the invention has a signal peptide and that the likely cleavage site for a NOV3 peptide is between positions 23 and 24, i.e., at the dash in the sequence SLG-CD.

TABLE 3B.
Encoded NOV3 protein sequence (SEQ ID NO:6)

MASPFALLMALVVLSCKSSCSLGCDLPETHSLDNRRTLMLLAQMSRISPSSCLMDRHDFGFPQEE
FDGNQFQKAPAISVLHELIQQIFNLFTTKDSSAAWDEDLLDQFCTELYQQLNDLEACVMQEERVG
ETPLMNADSILAVKKYFRRITLYLTEKKLGLCDWWVARAPILTTLSLSCNLQVTLGSKLCLLVHH
VTIAL

A search against the Patp database, a proprietary database that contains sequences published in patents and patent publications, yielded several homologous proteins shown in Table 3C.

TABLE 3C
Patp results for NOV3

| | | | Smallest |
|---|---|---|---|
| | | | Sum |
| Sequences producing | Reading | High | Prob |
| High-scoring Segment Pairs: | Frame | Score | P(N) |
| >patp: AAP30182 Human lymphoblastoid | +1 | 874 | 2.1e−87 |
| interferon | | | |
| >patp: AAP40126 Human CG-pBR | +1 | 874 | 2.1e−87 |
| 322/HLycIFN-8′1 | | | |
| >patp: AAP10020 Human interferon | +1 | 870 | 5.5e−87 |
| (IFN)-alpha-1 | | | |
| >patp: AAW53119 Human interferon-alpha | +1 | 870 | 5.5e−87 |
| type I | | | |

In a BLAST search of public sequence databases, it was found, for example, that the NOV3 nucleic acid sequence has 691 of 741 bases (93%) identical to a homo sapiens Interferon-alpha 13 precursor mRNA (GENBANK-ID: HSIFR18|acc:X00803). The full amino acid sequence of the protein of the invention was found to have 172 of 188 amino acid residues (91%) identical to, and 175 of 188 residues (93%) positive with, the 189 amino acid residue Interferon-alpha 13 precursor protein from homo sapiens (ptnr: SPTREMBL-ACC: Q14605). The global sequence homology (as defined by FASTA alignment with the full length sequence of this protein) is 91.534% amino acid homology and 91.005% amino acid identity.

NOV3 also has homology to the proteins shown in the BLASTP data in Table 3D.

TABLE 3D
BLAST results for NOV3

| Gene Index/ | | Length | Identity | Positives | |
|---|---|---|---|---|---|
| Identifier | Protein/Organism | (aa) | (%) | (%) | Expect |
| ptnr:SPTREMBL- | INTERFERON- | 189 | 172/188 | 175/188 | 3.3e- |
| ACC:Q14605 | ALPHA-13 | | (91%) | (93%) | 87 |
| | PRECURSOR | | | | |
| | [Homo sapiens] | | | | |
| ptnr:SWISSNEW- | INTERFERON ALPHA | 189 | 171/188 | 174/188 | 8.7e- |
| ACC:P01562 | 1/13 PRECURSOR | | (90%) | (92%) | 87 |
| | (INTERFERON ALPHA- | | | | |
| | D) (LEIF D) | | | | |
| | [Homo sapiens] | | | | |
| ptnr:SPTREMBL- | IFNA PROTEIN | 166 | 149/165 | 152/165 | 2.4e- |
| ACC:Q9UMJ3 | [Homo sapiens] | | (90%) | (92%) | 75 |

A multiple sequence alignment is given in Table 3E, with the NOV3 protein being shown on line 1 in Table 3E in a ClustalW analysis, and comparing the NOV3 protein with the related protein sequences shown in Table 3D. This BLASTP data is displayed graphically in the ClustalW in Table 3E.

TABLE 3E
ClustalW Analysis of NOV3

1) NOV3; SEQ ID NO:5
2) >Q14605/Interferon-alpha-13 precurson [Homo sapiens]; SEQ ID NO:29
3) >P01562/Interferon alpha 1/13 (interferon-alpha-D) [Homo sapiens]; SEQ ID NO:30
4) >Q9UMJ3/INFA protein [Homo sapiens]; SEQ ID NO:31

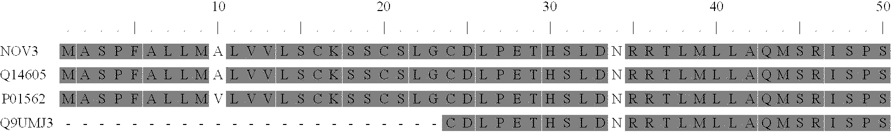

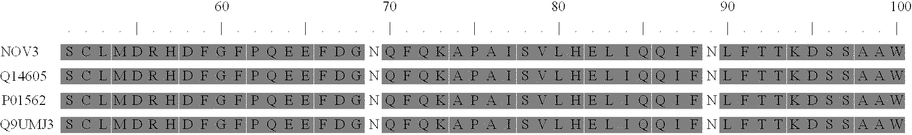

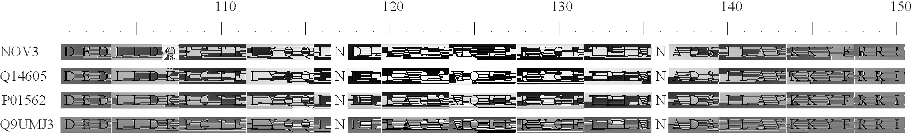

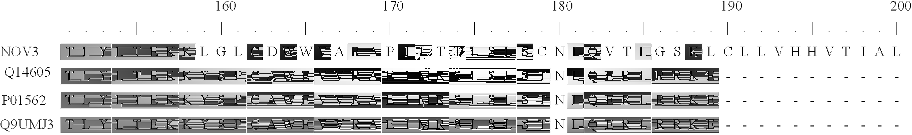

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 3F lists the domain description from DOMAIN analysis results against NOV3.

TABLE 3F
Domain Analysis of NOV3

| | Region of | | |
|---|---|---|---|
| Model | Homology | Score (bits) | E value |
| interferon | 1-189 | 373.4 | 2.4e−108 |

The presence of a protein region in NOV3 that is homologous to interferon alpha/beta domain (IPR00047 1) is consistent with the identification of NOV3 as an interferon-alpha-i 3-like protein. This indicates that the NOV3 sequence has properties similar to those of other proteins known to contain these domains.

The above defined information for this invention suggests that this interferon-alpha-13-like protein may function as a member of a “Interferon-alpha family.” Therefore, the nucleic acids and proteins of the invention are useful in potential therapeutic applications implicated in various pathologies/disorders described and/or other pathologies/disorders. For example, a cDNA encoding the interferon-alpha-13-like protein may be useful in gene therapy, and the interferon-alpha-13-like protein may be useful when administered to a subject in need thereof. By way of nonlimiting example, the compositions of the present invention will have efficacy for treatment of patients suffering cancer including but not limited to non-Hodgkin's lymphomas, renal cancer, hepatocellular carcinomas, and melanomas, myeloid leukemia, autoimmune and immune disorders, multiple sclerosis, inflammatory disorders, and Hepatitis C Virus. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

The novel nucleic acid encoding the interferon-alpha-13-like protein of the invention, or fragments thereof, may further be useful in diagnostic applications, wherein the presence or amount of the nucleic acid or the protein are to be assessed. The NOV3 polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen.

NOV4

A NOV4 polypeptide is an ADAM-like protein. The novel NOV4 nucleic acid sequences maps to the Unigene entry Hs.166003. Two alternative novel NOV4, NOV4a and NOV4b, nucleic acids and encoded polypeptides are provided.

NOV4a

A NOV4 variant is the novel NOV4a (alternatively referred to herein as SC30236456_EXT1), which includes the 2431 nucleotide sequence (SEQ ID NO:7) shown in Table 4A. The DNA sequence and protein sequence for a NOV4a gene or one of its splice forms was obtained by CAP extension of 30236456 using Spliced AC024958 83246-83346,84659-84760,89373-89450,47210-47290, 138925-139066,140841-140897,148510-148581,171191-171358,174324-174410,176236-176364,178206-178379,179845-179937, 180952-181146,181543-181647,182093-182281,188288-188368,189230-189307,4974-5075,5657-5758,58766-58701,57377-57274. A NOV4a ORF begins with a Kozak consensus ATG initiation codon at nucleotides 51-53 and ends with a TGA codon at nucleotides 2395-2397. Putative untranslated regions upstream from the initiation codon and downstream from the termination codon are underlined in Table 4A, and the start and stop codons are in bold letters.

TABLE 4A.
NOV4a Nucleotide Sequence (SEQ ID NO:7)

GAACTCCTTTTCTCAAGCACTTCTGCTCTCCTCTACCAGAATCACTCAGAATGCTTCCCGG
GTGTATATTCTTGATGATTTTACTCATTCCTCAGGTTAAAGAAAAGTTCATCCTTGGAGTAGAGGGT
CAACAACTGGTTCGTCCTAAAAAGCTTCCTCTGATACAGAAGCGAGATACTGGACACACCCATGATG
ATGACATAAAAACGTATGAAGAAGAATTGTTGTATGAAATAAAACTAAATAGAAAAACCTTAGTCCT
TCATCTTCTAAGATCCAGGAGGGAGTTCCTAGGCTCAAATTACAGTGAAACATTCTACTCCATGAAA
GGAGAAGCGTTCACCAGGCATCCTCAGATCATGGATCATTGTTTTTACCAAGGATCCATAGTACACG
AATATGATTCAGCTGCCAGTATCAGTACGTGTAATGGTCTAAGGGGATTCTTCAGAATAAACGACCA
AAGATACCTCATTGAACCAGTGAAATACTCAGATGAGGGAGAACATTTGGTGTTCAAATATAACCTG
AGGGTGCCGTATGGTGCCAATTATTCCTGTACAGAGCTTAATTTTACCAGAAAAACTGTTCCAGGGG
ATAATGAATCTGAAGAAGACTCCAAAATAAAACAGGGCATCCATGATGAAAAGTATGTTGAATTGTT
CATTGTTGCTGATGATACTGTGTATCGCAGAAATGGTCATCCTCACAATAAACTAAGGAACCGAATT
TGGGGAATGGTCAATTTTGTCAACATGATTTATAAAACCTTAAACATCCATGTGACGTTGGTTGGCA
TTGAAATATGGACACATGAAGATAAAATAGAACTATATTCAAATATAGAAACTACCTTATTGCGTTT
TTCATTTTGGCAAGAAAAGATCCTTAAAACACGGAAGGATTTTGATCATGTTGTATTACTCAGTGGG
AAGTGGCTCTACTCACATGTGCAAGGAATTTCTTATCCAGGGGGTATGTGCCTGCCCTATTATTCCA
CCAGTATCATTAAGGATCTTTTACCTGACACAAACATAATTGCAAACAGAATGGCACATCAACTGGG
GCATTACCTTGGGATGCAGCATGACGAGTTCCCATGCACCTGTCCTTCAGGAAAATGCGTGATGGAC
AGTGATGGAAGCATTCCTGCACTGAAATTCAGTAAATGCAGCCAAAACCAATACCACCAGTACTTGA
AGGATTATAAGCCAACATGCATGCTCAACATTCCATTTCCTTACAATTTTCATGATTTCCAATTTTG
TGGAAACAAGAAGTTGGATGAGGGTGAAGAGTGTGACTGTGGCCCTGCTCAGGAGTGTACTAATCCT
TGCTGTGATGCACACACATGTGTACTGAAGCCAGGATTTACTTGTGCAGAAGGAGAATGCTGTGAAT
CTTGTCAGATAAAAAAAGCAGGGTCCATATGCAGACCGGCGAAAGATGAATGTGATTTTCCTGAGAT
GTGCACTGGCCACTCGCCTGCCTGTCCTAAGGACCAGTTCAGGGTCAATGGATTTCCTTGCAAGAAC
TCAGAAGGCTACTGTTTCATGGGGAAATGTCCAACTCGTGAGGATCAGTGCTCTGAACTATTTGATG
ATGAGGCAATAGAGAGTCATGATATCTGCTACAAGATGAATACAAAAGGAAATAAATTTGGATACTG
CAAAAACAAGGAAAACAGATTTCTTCCCTGTGAGGAGAAGGATGTCAGATGTGGAAAGATCTACTGC
ACTGGAGGGGAGCTTTCCTCTCTCCTTGGAGAAGACAAGACTTATCACCTTAAGGATCCCCAGAAGA
ATGCTACTGTCAAATGCAAAACTATTTTTTTATACCATGATTCTACAGACATTGGCCTGGTGGCGTC
AGGAACAAAATGTGGAGAGGGAATGGTATGCAACAATGGTGAATGTCTAAACATGGAAAAGGTCTAT
ATCTCAACCAATTGCCCCTCTCAGTGCAATGAAAATCCTGTAGATGGCCACGGACTCCAGTGCCACT
GTGAGGAAGGACAGGCACCTGTAGCCTGTGAAGAAACCTTACATGTTACCAGTATCACCATCTTGGT
TGTTGTGCTTGTCCTGGTTATTGTCGGTATCGGAGTTCTTATACTATTAGTTCGTTACCGAAAATGT
ATCAAGTTGAAGCAAGTTCAGAGCCCACCTACAGAAACCCTGGGAGTGGAGAACAAAGGATACTTTG
GTGATGAGCAGCAGATAAGGACTGAGCCAATCCTGCCAGAAATTCATTTCCTAAATCAGAGAACTCC
AGAATCCTTGGAAAGCCTGCCCACTAGTTTTTCAAGTCCCCACTACATCACACTGAAACCTGCAAGT
AAAGATTCAAGAGGAATCGCAGATCCCAATCAAAGTGCCAAGTGGTAGGTTACCCTGACAGATAGTA
CCTCCCTTTTTTATTTTTCAAATGC

The NOV4a polypeptide (SEQ ID NO: 8) encoded by SEQ ID NO:7 is 778 amino acid residues in length, has a molecular weight of 88471.2 Daltons, and is presented using the one-letter amino acid code in Table 4B. The Psort profile for both NOV4a and NOV4b predicts that these sequences are likely to be localized at the endoplasmic reticulum (membrane) with a certainty of 0.9325. The Signal P predicts a likely cleavage site for a NOV4 peptide is between positions 18 and 19, i.e., at the dash in the sequence VKE-KF.

TABLE 4B.
NOV4a protein sequence (SEQ ID NO:8)

MLPGCIFLMILLIPQVKEKFILGVEGQQLVRPKKLPLIQKRDTGHTHDDDIKTYEEELLYEIKLNRK
TLVLHLLRSRREFLGSNYSETFYSMKGEAFTRHPQIMDHCFYQGSIVHEYDSAASISTCNGLRGFFR
INDQRYLIEPVKYSDEGEHLVFKYNLRVPYGANYSCTELNFTRKTVPGDNESEEDSKIKQGIHDEKY
VELFIVADDTVYRRNGHPHNKLRNRIWGMVNFVNMIYKTLNIHVTLVGIEIWTHEDKIELYSNIETT
LLRFSFWQEKILKTRKDFDHVVLLSGKWLYSHVQGISYPGGMCLPYYSTSIIKDLLPDTNIIANRMA
HQLGHNLGMQHDEFPCTCPSGKCVMDSDGSIPALKFSKCSQNQYHQYLKDYKPTCMLNIPFPYNFHD
FQFCGNKKLDEGEECDCGPAQECTNPCCDAHTCVLKPGFTCAEGECCESCQIKKAGSICRPAKDECD
FPEMCTGHSPACPKDQFRVNGFPCKNSEGYCFMGKCPTREDQCSELFDDEAIESHDICYKMNTKGNK
FGYCKNKENRFLPCEEKDVRCGKIYCTGGELSSLLGEDKTYHLKDPQKNATVKCKTIFLYHDSTDIG
LVASGTKCGEGMVCNNGECLNMEKVYISTNCPSQCNENPVDGHGLQCHCEEGQAPVACEETLHVTSI
TILVVVLVLVIVGIGVLILLVRYRKCIKLKQVQSPPTETLGVENKGYFGDEQQIRTEPILPEIHFLN
QRTPESLESLPTSFSSPHYITLKPASKDSRGIADPNQSAKW

NOV4b

Alternatively, a NOV4 variant is the novel NOV4b (alternatively referred to herein as AC024958_A), which includes the 2434 nucleotide sequence (SEQ ID NO:9) shown in Table 4C. NOV4b was created by splicing together regions of the genomic clone AC024958 (Spliced regions 83246-83346,84659-84760,89373-89450,47210-47290,111495-111572,138875-139066,140841-140897,148510-148581,171191-171358,174324-174410, 176236-176364,178206-178379,179845-179937,180952-181146,181543-181647, 182093-182281,188288-188368,189230-189307,4974-5075,5657-5758,58766-58701,57377-57274). The NOV4b ORF begins with a Kozak consensus ATG initiation codon at nucleotides 51-53 and ends with a TGA codon at nucleotides 2398-2400. Putative untranslated regions upstream from the initiation codon and downstream from the termination codon are underlined in Table 4C, and the start and stop codons are in bold letters.

TABLE 4C.
NOV4b Nucleotide Sequence (SEQ ID NO:9)

GAACTCCTTTTCTCAAGCACTTCTGCTCTCCTCTACCAGAATCACTCAGAATGCTTCCCGGGTGTAT
ATTCTTGATGATTTTACTCATTCCTCAGGTTAAAGAAAAGTTCATCCTTGGAGTAGAGGGTCAACAA
CTGGTTCGTCCTAAAAAGCTTCCTCTGATACAGAAGCGAGATACTGGACACACCCATGATGATGACA
TAAAAACGTATGAAGAAGAATTGTTGTATGAAATAAAACTAAATAGuAAAAACCTTAGTCCTTCATCT
TCTAAGATCCAGGAGGGAGTTCCTAGGCTCAAATTACAGTGAAACATTCTACTCCATGAAAGGAGAA
GCGTTCACCAGGCATCCTCAGATCATGGAACACTGTTACTATAAAGGAAACATCCTAAATGAAAAGA
ATTCTGTTGCCAGCATCAGTACTTGTGACGGGTTGAGGAGGGGATTCTTCAGAATAAACGACCAAAG
ATACCTCATTGAACCAGTGAAATACTCAGATGAGGGAGAACATTTGGTGTTCAAATATAACCTGAGG
GTGCCGTATGGTGCCAATTATTCCTGTACAGAGCTTAATTTTACCAGAAAAACTGTTCCAGGGGATA
ATGAATCTGAAGAAGACTCCAAAATAAAACAGGGCATCCATGATGAAAAGTATGTTGAATTGTTCAT
TGTTGCTGATGATACTGTGTATCGCAGAAATGGTCATCCTCACAATAAACTAAGGAACCGAATTTGG
GGAATGGTCAATTTTGTCAACATGATTTATAAAACCTTAAACATCCATGTGACGTTGGTTGGCATTG
AAATATGGACACATGAAGATAAAATAGAACTATATTCAAATATAGAAACTACCTTATTGCGTTTTTC
ATTTTGGCAAGAAAAGATCCTTAAAACACGGAAGGATTTTGATCATGTTGTATTACTCAGTGGGAAG
TGGCTCTACTCACATGTGCAAGGAATTTCTTATCCAGGGGGTATGTGCCTGCCCTATTATTCCACCA
GTATCATTAAGGATCTTTTACCTGACACAAACATAATTGCAAACAGAATGGCACATCAACTGGGGCA
TAACCTTGGGATGCAGCATGACGAGTTCCCATGCACCTGTCCTTCAGGAAAATGCGTGATGGACAGT
GATGGAAGCATTCCTGCACTGAAATTCAGTAAATGCAGCCAAAACCAATACCACCAGTACTTGAAGG
ATTATAAGCCAACATGCATGCTCAACATTCCATTTCCTTACAATTTTCATGATTTCCAATTTTGTGG
AAACAAGAAGTTGGATGAGGGTGAAGAGTGTGACTGTGGCCCTGCTCAGGAGTGTACTAATCCTTGC
TGTGATGCACACACATGTGTACTGAAGCCAGGATTTACTTGTGCAGAAGGAGAATGCTGTGAATCTT
GTCAGATAAAAAAAGCAGGGTCCATATGCAGACCGGCGAAAGATGAATGTGATTTTCCTGAGATGTG
CACTGGCCACTCGCCTGCCTGTCCTAAGGACCAGTTCAGGGTCAATGGATTTCCTTGCAAGAACTCA
GAAGGCTACTGTTTCATGGGGAAATGTCCAACTCGTGAGGATCAGTGCTCTGAACTATTTGATGATG
AGGCAATAGAGAGTCATGATATCTGCTACAAGATGAATACAAAAGGAAATAAATTTGGATACTGCAA
AAACAAGGAAAACAGATTTCTTCCCTGTGAGGAGAAGGATGTCAGATGTGGAAAGATCTACTGCACT
GGAGGGGAGCTTTCCTCTCTCCTTGGAGAAGACAAGACTTATCACCTTAAGGATCCCCAGAAGAATG
CTACTGTCAAATGCAAAACTATTTTTTTATACCATGATTCTACAGACATTGGCCTGGTGGCGTCAGG
AACAAAATGTGGAGAGGGAATGGTATGCAACAATGGTGAATGTCTAAACATGGAAAAGGTCTATATC
TCAACCAATTGCCCCTCTCAGTGCAATGAAAATCCTGTAGATGGCCACGGACTCCAGTGCCACTGTG
AGGAAGGACAGGCACCTGTAGCCTGTGAAGAAACCTTACATGTTACCAGTATCACCATCTTGGTTGT
TGTGCTTGTCCTGGTTATTGTCGGTATCGGAGTTCTTATACTATTAGTTCGTTACCGAAAATGTATC
AAGTTGAAGCAAGTTCAGAGCCCACCTACAGAAACCCTGGGAGTGGAGAACAAAGGATACTTTGGTG
ATGAGCAGCAGATAAGGACTGAGCCAATCCTGCCAGAAATTCATTTCCTAAATCAGAGAACTCCAGA
ATCCTTGGAAAGCCTGCCCACTAGTTTTTCAAGTCCCCACTACATCACACTGAAACCTGCAAGTAAA
GATTCAAGAGGAATCGCAGATCCCAATCAAAGTGCCAAGTGGTAGGTTACCCTGACAGATAGTACCT
CCCTTTTTTATTTTTCAAATGC

The NOV4b protein (SEQ ID NO: 10) encoded by SEQ ID NO:9 is 779 amino acid residues in length, has a molecular weight of 88668.5 Daltons, and is presented using the one-letter code in Table 4D.

TABLE 4D.
NOV4b protein sequence (SEQ ID NO:1O)

MLPGCIFLMILLIPQVKEKFILGVEGQQLVRPKKLPLIQKRDTGHTHDDDIKTYEEELLYEIKLNRK
TLVLHLLRSRREFLGSNYSETFYSMKGEAFTRHPQIMEHCYYKGNILNEKNSVASISTCDGLRRGFF
RINDQRYLIEPVKYSDEGEHLVFKYNLRVPYGANYSCTELNFTRKTVPGDNESEEDSKIKQGIHDEK
YVELFIVADDTVYRRNGHPHNKLRNRIWGMVNFVNMIYKTLNIHVTLVGIEIWTHEDKIELYSNIET
TLLRFSFWQEKILKTRKDFDHVVLLSGKWLYSHVQGISYPGGMCLPYYSTSIIKDLLPDTNIIANRM
AHQLGHNLGMQHDEFPCTCPSGKCVMDSDGSIPALKFSKCSQNQYHQYLKDYKPTCMLNIPFPYNFH
DFQFCGNKKLDEGEECDCGPAQECTNPCCDAHTCVLKPGFTCAEGECCESCQIKKAGSICRPAKDEC
DFPEMCTGHSPACPKDQFRVNGFPCKNSEGYCFMGKCPTREDQCSELFDDEAIESHDICYKMNTKGN
KFGYCKNKENRFLPCEEKDVRCGKIYCTGGELSSLLGEDKTYHLKDPQKNATVKCKTIFLYHDSTDI
GLVASGTKCGEGMVCNNGECLNMEKVYISTNCPSQCNENPVDGHGLQCHCEEGQAPVACEETLHVTS
ITILVVVLVLVIVGIGVLILLVRYRKCIKLKQVQSPPTETLGVENKGYFGDEQQIRTEPILPEIHFL
NQRTPESLESLPTSFSSPHYITLKPASKDSRGIADPNQSAKW

NOV4 Clones

Unless specifically addressed as NOV4a or NOV4b, any reference to NOV4 is assumed to encompass all variants. Residue differences between any NOV4 variant sequences herein are written to show the residue in the “a” variant, the residue position with respect to the “a” variant, and the residue in the “b” variant. For example, the NOV4 nucleic acid sequences differ at the following position: 441 to 443. The NOV4 polypeptides differ only at one residue, namely amino acid residue 118. The homologies shown above are shared by NOV4b insofar as NOV4a and 1b are homologous as shown in Table 4E and Table 4G.

A search against the Patp database, a proprietary database that contains sequences published in patents and patent publications, yielded several homologous proteins shown in Table 4E.

TABLE 4E
Patp results for NOV4

| | | | Smallest |
|---|---|---|---|
| | | | Sum |
| Sequences producing | Reading | High | Prob |
| High-scoring Segment Pairs: | Frame | Score | P(N) |
| >patp: AAB64744 Gene 16 human secreted | +1 | 2017 | 1.6e−208 |
| protein | | | |
| >patp: AAW90855 Human ADAM protein | +1 | 1446 | 5.0e−148 |
| #2 | | | |
| >patp: AAW90865 Human ADAM protein | +1 | 1446 | 5.0e−148 |
| #4 | | | |
| >patp: AAW90865 Human ADAM protein | +1 | 1123 | 8.5e−114 |
| #1 | | | |
| >patp: AAW90864 Human ADAM protein | +1 | 1123 | 8.5e−114 |
| #3 | | | |

In a BLAST search of public sequence databases, it was found, for example, that the nucleic acid sequence of this invention (SC30236456_EXT1) has 2264 of 2364 bases (95%) identical to a macaque mRNA (GENBANK-ID: X66139). The full amino acid sequence of the protein of the invention (SC30236456_EXT1) was found to have 727 of 777 amino acid residues (95%) identical to, and 739 of 777 residues (95%) similar to, the 778 amino acid residue protein from macaque (ptnr:SPTREMBL-ACC:Q28475).

Similarly for the alternative splice form, in a search of sequence databases, it was found, for example, that the nucleic acid sequence of this invention (AC024958_A) has 2211 of 2367 bases (93%) identical to a macaque mRNA (GENBANK-ID: X66139). The full amino acid sequence of the protein of the invention (AC024958_A) was found to have 727 of 777 amino acid residues (95%) identical to, and 739 of 777 residues (95%) similar to, the 778 amino acid residue protein from macaque (ptnr:SPTREMBL-ACC:Q28475).

Additional BLAST results are shown in Table 4F.

TABLE 4F
BLAST results for NOV4

| Gene Index/ | | Length | Identity | Positives | |
|---|---|---|---|---|---|
| Identifier | Protein/Organism | (aa) | (%) | (%) | Expect |
| ptnr:SPTREMBL- | A DISINTEGRIN AND | 754 | 735/746 | 739/746 | 0.0 |
| ACC:Q9R2U9 | METALLOPROTEINASE | | (98%) | (99%) | |
| | 7 | | | | |
| | [Homo sapiens] | | | | |
| ptnr:SPTREMBL- | EPIDIDYMAL APICAL | 776 | 727/777 | 750/777 | 0.0 |
| ACC:Q28475 | PROTEIN I- | | (93%) | (96%) | |
| | PRECURSOR | | | | |
| | [Macaca | | | | |
| | fascicularis | | | | |
| ptnr:SPTREMBL- | SPERM MATURATION- | 588 | 569/580 | 572/580 | 0.0 |
| ACC:075959 | RELATED | | (98%) | (98%) | |
| | GLYCOPROTEIN GP-83 | | | | |
| | [Home sapiens] | | | | |
| ptnr:SPTREMBL- | A DISINTEGRIN AND | 788 | 531/775 | 640/775 | 9.6e- |
| ACC:035227 | METALLOPROTEASE | | (68%) | (82%) | 310 |
| | DOMAIN (ADAM 7) | | | | |
| | [Mus museulus] | | | | |

A multiple sequence alignment is given in Table 4G, with the NOV4 protein of the invention being shown on line 1, in a ClustalW analysis comparing NOV4 with related protein sequences disclosed in Table 4F.

TABLE 4G
Information for the ClustalW proteins:

1. >NOV4a; SEQ ID NO:7
2. >NOV4b; SEQ ID NO:9
3. >Q9H2U9/A disintegrin and metalloproteinase 7 [Homo sapiens]; SEQ ID NO:32
4. >Q28475/Epididymal apical protein I precursor [Macaca fascicularis]; SEQ ID NO:33
5. >O75959/Sperm maturation-related glycoprotein GP-83 [Homo sapiens]; SEQ ID NO:34
6. >O35227/A disintegrin and metalloproteinase domain (ADAM 7) [Mus musculus]; SEQ ID NO:35

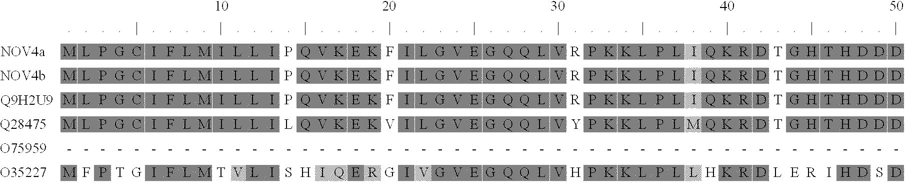

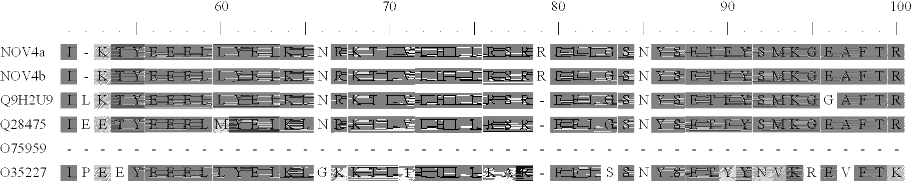

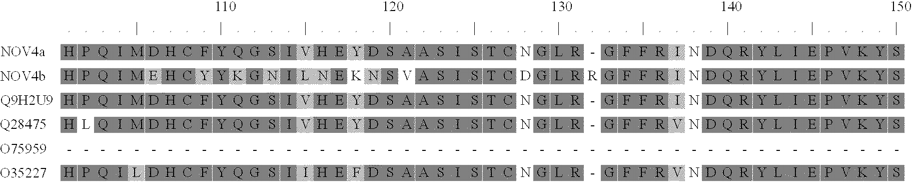

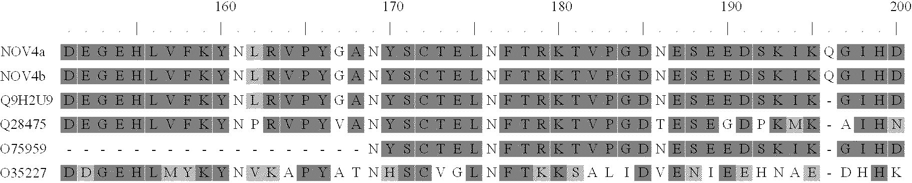

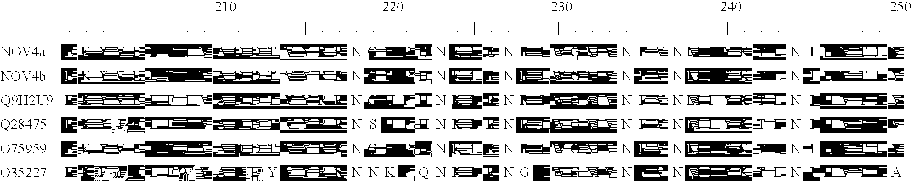

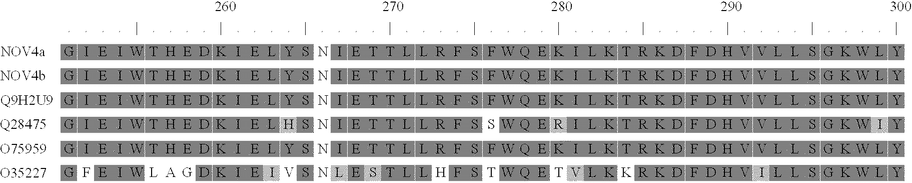

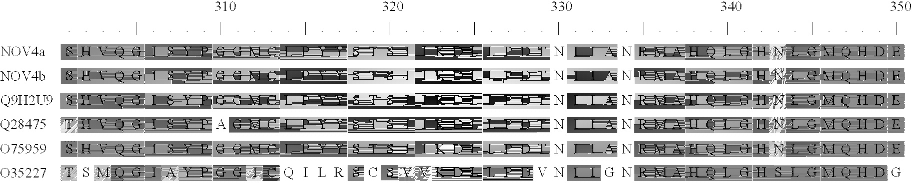

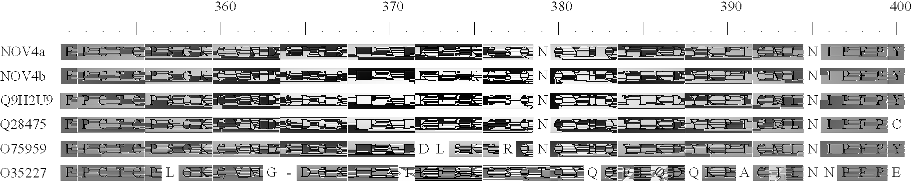

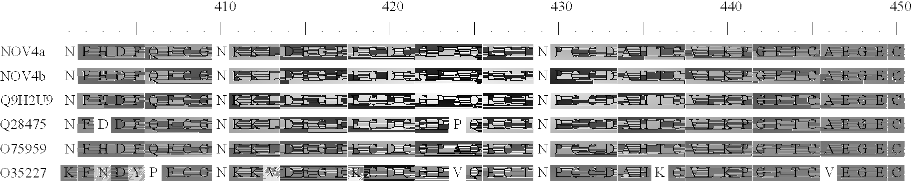

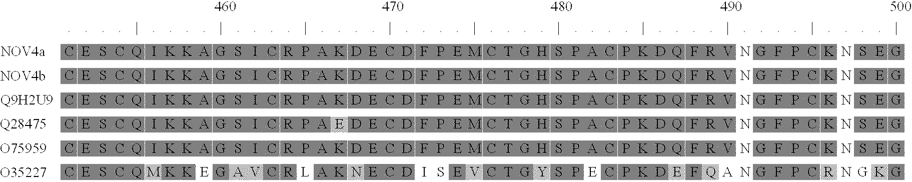

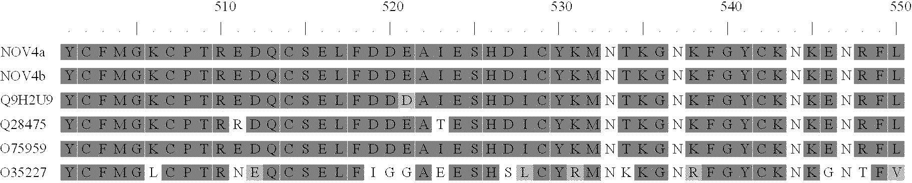

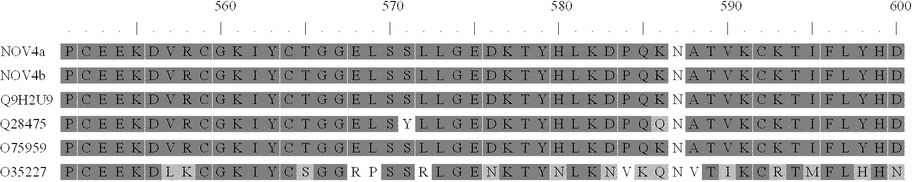

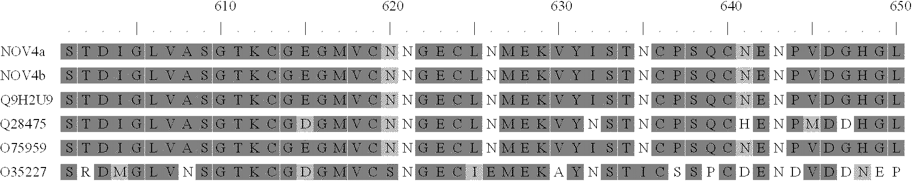

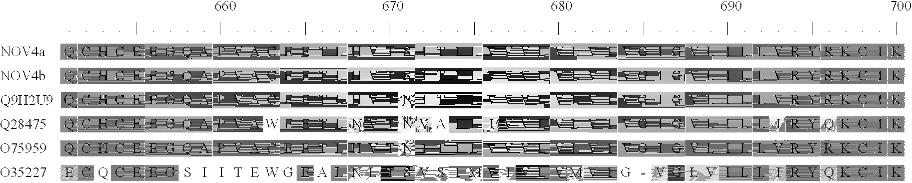

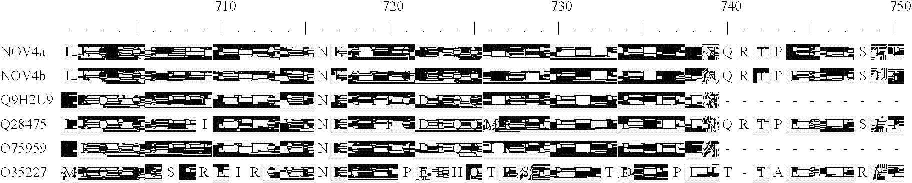

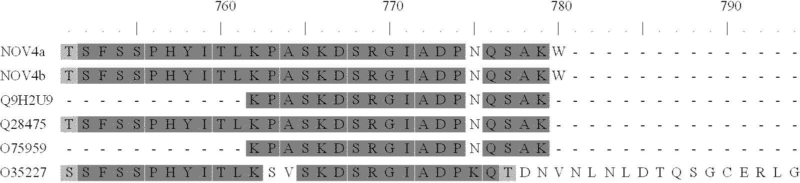

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 4H lists the domain description from DOMAIN analysis results against NOV4.

TABLE 4H
Domain Analysis of NOV4

| | Region of | | |
|---|---|---|---|
| Model | Homology | Score (bits) | E value |
| Pep_M12B_propep | 73-189 | 149.5 | 6e-41 |
| Reprolysin | 201-398 | 358.8 | 5.8e-104 |
| disintegrin | 413-488 | 96.2 | 6.8e-31 |

The characteristic feature of ADAM proteins is their unique domain organization, which includes the presence of a pro-, metalloprotease and disintegrin-like domains. The presence of protein regions on NOV4 that are homologous to the Pep_M12B_propep domain (IPR002870), the reprolysin (M12B) family zinc metalloproteinase domain (reprolysin; IPR001590), and the disintegrin domain (IPR001762) is consistent with the organization of members of the ADAM Protein Family. This indicates that the NOV4 sequence has properties similar to those of other ADAM-like proteins known to contain these domains.

The ADAM-like proteins disclosed in this invention are expressed in at least the following tissues: testis and several cell lines Caco2, MCF-7, TSC fibroblasts, HUVEC, HUAEC, OVCAR-3, IGROV-1, BT549, HS528T, Metastatic A5(Thyroid cancer), Follicular Adenoma (Thyroid cancer). Accordingly, the NOV4 nucleic acid and polypeptide are useful in identifying tissues. Specifically, the NOV4 nucleic acid and polypeptide in the diagnosis of thyroid cancer. The expression pattern, map location, domain analysis, and protein similarity information for the invention suggest that this NOV4 (SC30236456_EXT1 and AC024958 A) may function an ADAM-like proteins. The NOV4 nucleic acids and proteins of the invention, therefore, are useful in potential therapeutic applications implicated, for example but not limited to, in various pathologies/disorders as described below and/or other pathologies/disorders. Potential therapeutic uses for the invention(s) are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the ADAM-like protein may be useful in gene therapy, and the ADAM-like protein may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, hyperthyroidism, hypothyroidism, endometriosis, fertility, angiogenesis, hypertension, stroke, ischemia, atherosclerosis, aneurysms, stroke, bleeding disorders. The novel nucleic acid encoding the ADAM-like protein, and the ADAM-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV5

A protein of the invention, referred to herein as NOV5, is an Ankyrin Repeat-containing (ASB-1)-like protein. The ASB-1-like gene disclosed in this invention maps to chromosome X. This assignment was made using mapping information associated with genomic clones, public genes and ESTs sharing sequence identity with the disclosed sequence and CuraGen Corporation's Electronic Northern bioinformatic tool. Two alternative novel NOV5, NOV5a and NOV5b, nucleic acids and encoded polypeptides are provided.

NOV5a

A NOV5 variant is the novel NOV5a (alternatively referred to herein as SC_{—}86058175_A), which includes the 1069 nucleotide sequence (SEQ ID NO:11) shown in Table 5A. The nucleic acid sequence was predicted from the genomic file Sequencing Center accession number:#ba403e24 by homology to a known ASB-1 or homolog. A NOV5a ORF begins with a Kozak consensus ATG initiation codon at nucleotides 6-8 and ends with a TAA codon at nucleotides 1059-61. Putative untranslated regions upstream from the initiation codon and downstream from the termination codon are underlined in Table 5A, and the start and stop codons are in bold letters.

TABLE 5A
NOV5 Nucleotide Sequence (SEQ ID NO:11)

CATGAATGAGTAGCCTGATGGTGAAATGGAGGGAGATTTCAAGGAGCGTGCATGGTCAGGCTTTTGA
TGGGTACCCTCATATGAGAATAGTTCTCCAATTAGCCAAGATGAACCTCATGGACATCACCAAGATC
TTCTCCCTCCTGCAGCCCGACAAGGAGGAGGAGGACACTGACACAGAGGAGAAGCAGGCTCTCAATC
AAGCAGTGTATGACAACGACTCCTATACTTTGGACCAGCTTTTGCGCCAGGAGCGTTACAAACGTTT
CATCAACAGCAGGAGTGGCTGGGGTGTTCCTGGGACACCCTTGCGCTTGGCTGCTTCTTATGGCCAC
TTGAGCTGTTTGCAAGTCCTCTTAGCCCATGGTGCTGATGTTGACAGCTTGGATGTCAAGGCACAGA
CGCCACTTTTCACTGCTGTCAGTCATGGCCATCTGGACTGTGTACGTGTGCTTTTGGAAGCTGGTGC
CTCTCCTGGTGGTAGCATCTACAACAACTGTTCTCCCGTGCTCACAGCTGCCCGTGATGGTGCTGTT
GCTATCCTGCAGGAGCTCCTAGACCATGGTGCAGAGGCCAACGTCAAAGCTAAACTACCAGTCTGGG
CATCAAACATAGCTTCATGTTCTGGCCCCCTCTATTTGGCCGCAGTCTACGGGCACCTGGACTGTTT
CCGCCTGCTTTTGCTCCACGGGGCAGACCCTGACTACAACTGCACTGACCAGGGCCTATTGGCTCGT
GTCCCAAGACCCCGCACCCTCCTTGAAATCTGCCTCCATCATAATTGTGAGCCAGAGTATATCCAGC
TGTTAATCGATTTTGGTGCTAATATCTACCTTCCATCTCTCTCCCTTGACCTGACCTCACAAGATGA
TAAAGGCATTGCATTGCTGCTACAGGCCCGAGGTGAGCTGTTTCTTCTTGCTGTAGCCACTCCACGG
TCACTTCTATCACAGGTCCGTTTAGTCGTCCGCAGAGCCTTGTGCCAGGCTGGCCAGCCACAAGCCA
TCAACCAGCTGGATATTCCTCCCATGTTGATTAGCTACCTAAAACACCAACTGTAATCTTGCAG

The NOV5a polypeptide (SEQ ID NO: 12) encoded by SEQ ID NO: 11 is 351 amino acid residues in length, has a molecular weight of 38740 Daltons, and is presented using the one-letter amino acid code in Table 5B.

TABLE 5B
NOV5a protein sequence (SEQ ID NO:2)

MSSLMVKWREISRSVHGQAFDGYPHMRIVLQLAKMNLMDITKIFSLLQPDKEEEDTDTEEKQALNQA
VYDNDSYTLDQLLRQERYKRFINSRSGWGVPGTPLRLAASYGHLSCLQVLLAHGADVDSLDVKAQTP
LFTAVSHGHLDCVRVLLEAGASPGGSIYNNCSPVLTAARDGAVAILQELLDHGAEANVKAKLPVWAS
NIASCSGPLYLAAVYGHLDCFRLLLLHGADPDYNCTDQGLLARVPRPRTLLEICLHHNCEPEYIQLL
IDFGANIYLPSLSLDLTSQDDKGIALLLQARGELFLLAVATPRSLLSQVRLVVRRALCQAGQPQAIN
QLDIPPMLISYLKHQL

NOV5b

A NOV5 variant is the novel NOV5b (alternatively referred to herein as CG57600-01), which includes the 1222 nucleotide sequence (SEQ ID NO:13) shown in Table 5C. NOV5b differs from NOV5a in being a splice variant with 17 extra amino acids toward the N-terminal region, 8 amino acids missing toward the C-terminal region and having one different amino acid. NOV5b was created by laboratory cloning of cDNA fragments, by in silico prediction of the sequence. Complimentary DNA fragments covering either the full length of the DNA sequence, or part of the sequence, or both, were cloned. In silico prediction based on sequences available in provided either the full length DNA sequence, or some portion thereof. The NOV5b ORF begins with a Kozak consensus ATG initiation codon at nucleotides 6-8 and ends with a TAA codon at nucleotides 1086-1088. Putative untranslated regions upstream from the initiation codon and downstream from the termination codon are underlined in Table 5C, and the start and stop codons are in bold letters.

TABLE 5C
NOV5b Nucleotide Sequence (SEQ ID NO:13)

CATGAATGAGTAGCCTGATGGTGAAATGGAGGGAGATTTCAAGGAGCGTGCATGGTCAGGCTTTTGA
TGGGTACCCTCATTCTCTCCCACTTGTTTGTCACCCACAGATCTGGCATTTCCTTGTGCTCATAATG
AGAATAGTTCTCCAATTAGCCAAGATGAACCTCATGGACATCACCAAGATCTTCTCCCTCCTGCAGC
CCGACAAGGAGGAGGAGGACACTGACACAGAGGAGAAGCAGGCTCTCAATCAAGCAGTGTATGACAA
CGACTCCTATACTTTGGACCAGCTTTTGCGCCAGGAGCGTTACAAACGTTTCATCAACAGCAGGAGT
GGCTGGGGTGTTCCTGGGACACCCTTGCGCTTGGCTGCTTCTTATGGCCACTTGAGCTGTTTGCAAG
TCCTCTTAGCCCATGGTGCTGATGTTGACAGCTTGGATGTCAAGGCACAGACGCCACTTTTCACTGC
TGTCAGTCATGGCCATCTGGACTGTGTACGTGTGCTTTTGGAAGCTGGTGCCTCTCCTGGTGGTAGC
ATCTACAACAACTGTTCTCCCGTGCTCACAGCTGCCCGTGATAGTGCTGTTGCTATCCTGCAGGAGC
TCCTAGACCATGGTGCAGAGGCCAACGTCAAAGCTAAACTACCAGTCTGGGCATCAAACATAGCTTC
ATGTTCTGGCCCCCTCTATTTGGCCGCAGTCTACGGGCACCTGGACTGTTTCCGCCTGCTTTTGCTC
CACGGGGCAGACCCTGACTACAACTGCACTGACCAGGGCCTATTGGCTCGTGTCCCAAGACCCCGCA
CCCTCCTTGAAATCTGCCTCCATCATAATTGTGAGCCAGAGTATATCCAGCTGTTAATCGATTTTGG
TGCTAATATCTACCTTCCATCTCTCTCCCTTGACCTGACCTCACAAGATGATAAAGGCATTGCATTG
CTGCTACAGGCCCGAGCCACTCCACGGTCACTTCTATCACAGGTCCGTTTAGTCGTCCGCAGAGCCT
TGTGCCAGGCTGGCCAGCCACAAGCCATCAACCAGCTGGATATTCCTCCCATGTTGATTAGCTACCT
AAAACACCAACTGTAATCTTGCAGTCTCCCCAGGAACTTATGATGCCTCCGAAAACCACCTGGGGAC
TCACGTAGCTGGAGAGCATTACAGCCTCATCCACTTACCTGGAGCTGCTCTCCTGTATTATCCTCCA
CAATAAAATTCTCCAG

The NOV5b protein (SEQ ID NO: 14) encoded by SEQ ID NO: 13 is 360 amino acid residues in length, has a molecular weight of 39924.5 Daltons, and is presented using the one-letter code in Table 5D. The Psort profile for both NOV 5a and NOV5b predicts that these sequences are likely to be localized to the mitochondrial matrix space with a certainty of 0.5160. Based upon SignalP analysis, NOV5b peptide likely contains a cleavage site between positions 50 and 51, i.e., at the dash in the sequence QLA-KM.

TABLE 5D
NOV5b protein sequence (SEQ ID NO:14)

MSSLMVKWREISRSVHGQAFDGYPHSLPLVCHPQIWHFLVLIMRIVLQLAKMNLMDITKIFSLLQPD
KEEEDTDTEEKQALNQAVYDNDSYTLDQLLRQERYKRFINSRSGWGVPGTPLRLAASYGHLSCLQVL
LAHGADVDSLDVKAQTPLFTAVSHGHLDCVRVLLEAGASPGGSIYNNCSPVLTAARDSAVAILQELL
DHGAEANVKAKLPVWASNIASCSGPLYLAAVYGHLDCFRLLLLHGADPDYNCTDQGLLARVPRPRTL
LEICLHHNCEPEYIQLLIDFGANIYLPSLSLDLTSQDDKGIALLLQARATPRSLLSQVRLVRRALC
QAGQPQAINQLDIPPMLISYLKHQL

NOV5 Clones

Unless specifically addressed as NOV5a or NOV5b, any reference to NOV5 is assumed to encompass all variants. Residue differences between any NOV5 variant sequences herein are written to show the residue in the “a” variant, the residue position with respect to the “a” variant, and the residue in the “b” variant. For example, NOV5b differs from NOV5a in being a splice variant with 17 extra amino acids toward the N-terminal region, 8 amino acids missing toward the C-terminal region and having one different amino acid. The homologies shown above are shared by NOV5a insofar as NOV5a and NOV5b are homologous as shown in Table 5E and Table 5G.

A search against the Patp database, a proprietary database that contains sequences published in patents and patent publications, yielded several homologous proteins shown in Table 5E.

TABLE 5E
Patp results for NOV5

| | | | Smallest |
|---|---|---|---|
| | | | Sum |
| Sequences producing | Reading | High | Prob |
| High-scoring Segment Pairs: | Frame | Score | P(N) |
| >patp: AAW62621 Mus musculus SOCS7 | +1 | 460 | 1.5e−43 |
| protein | | | |
| >patp: AAY53886 Human cytokine | +1 | 200 | 7.8e−15 |
| suppressor protein HSCOP-6 | | | |
| >patp: AAB95322 Human protein sequence | +1 | 194 | 9.4e−14 |
| SEQ ID NO: 17580 | | | |
| >patp: AAB93879 Human protein sequence | +1 | 203 | 9.4e−14 |
| SEQ ID NO: 13792 | | | |

In a BLAST search of public sequence databases, it was found, for example, that the nucleic acid sequence has 192 of 301 bases (63%) identical to Mus musculus ankyrin repeat-containing protein ASB-1 mRNA (GENEBANK-ID: AF155352). The full amino acid sequence of the protein of the invention was found to have 117 of 299 amino acid residues (39%) identical to, and 175 of 299 residues (58%) positive with, the 355 amino acid residues of ASB-1 PROTEIN protein from Homo sapiens (SPTREMBL-ACC:Q9Y576). The global sequence homology (as defined by FASTA alignment with the full-length sequence of this protein) is 51% amino acid homology and 40% amino acid identity.

Additional BLAST results are shown in Table 5F.

TABLE 5F
BLAST results for NOV5

| Gene Index/ | | Length | Identity | Positives | |
|---|---|---|---|---|---|
| Identifier | Protein/Organism | (aa) | (%) | (%) | Expect |
| ptnr:SPTREMBL- | 2310036C05RIK | 308 | 273/316 | 290/316 | 3.2e- |
| ACC:Q9D738 | PROTEIN | | (86%) | (91%) | 144 |
| | [Mus musculus] | | | | |
| ptnr:SPTREMBL- | ASB-1 PROTEIN | 335 | 117/299 | 175/299 | 3.0e- |
| ACC:Q9Y576 | [Homo sapiens] | | (39%) | (58%) | 45 |
| ptnr:SPTREMBL- | Ankyrin repeat- | 336 | 112/295 | 172/295 | 1.9e- |
| ACC:Q9WV74 | containing protein | | (37%) | (58%) | 43 |
| | ASB-1 | | | | |
| | [Mus musculus] | | | | |
| ptnr:SPTREMBL- | 1700029O08RIK | 327 | 109/271 | 161/271 | 3.1e- |
| ACC:Q9D9S9 | PROTEIN | | (40%) | (59%) | 43 |
| | [Mus musculus] | | | | |
| ptnr:SPTREMSL- | KIAA1146 PROTEIN | 271 | 106/270 | 160/270 | 1.8e- |
| ACC:Q9ULS4 | [Homo sapiens] | | (39%) | (59%) | 40 |

A multiple sequence alignment is given in Table 5G, with the NOV5 protein of the invention being shown on line 1, in a ClustalW analysis comparing NOV5 with related protein sequences disclosed in Table 5F.

TABLE 5G
Information for the ClustalW proteins:

1. >NOV5a; SEQ ID NO:11
2. >NOV5b; SEQ ID NO:13
3. >QD738/2310036C05RIK protein [Mus musculus]; SEQ ID NO:36
4. >Q9Y576/ASB-1 protein [Homo sapiens]; SEQ ID NO:37
5. >Q9WV74/Ankyrin repeat-containing protein ASB-1 [Mus musculus]; SEQ ID NO:38
6. >Q9D9S9/1700029O08RIK protein [Mus musculus]; SEQ ID NO:39
7. >Q9ULS4/KIAA1146 protein [Homo sapiens]; SEQ ID NO:40

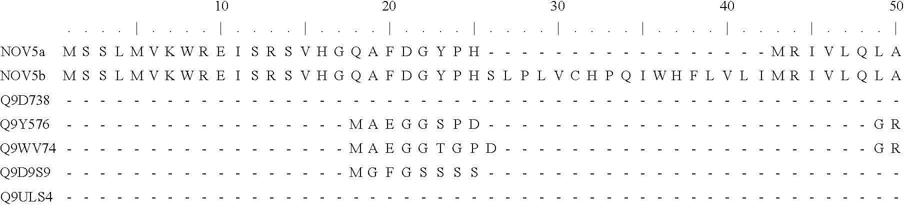

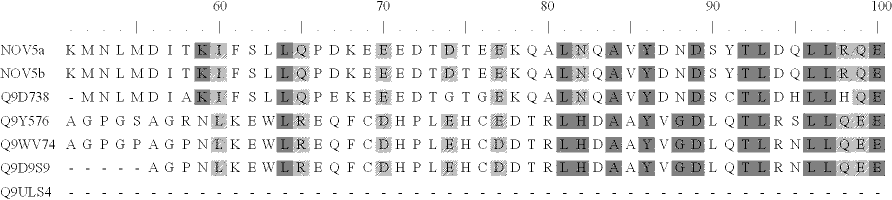

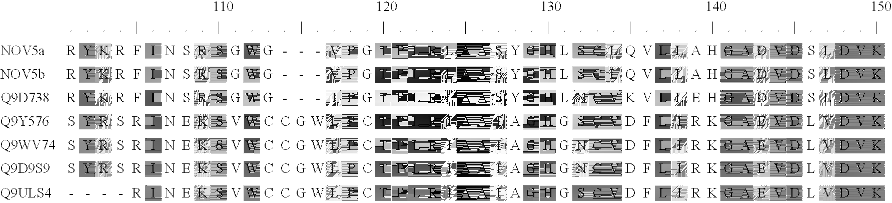

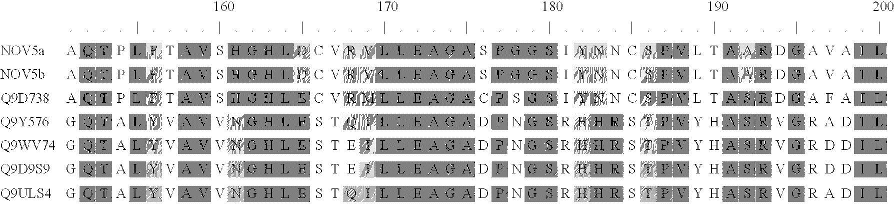

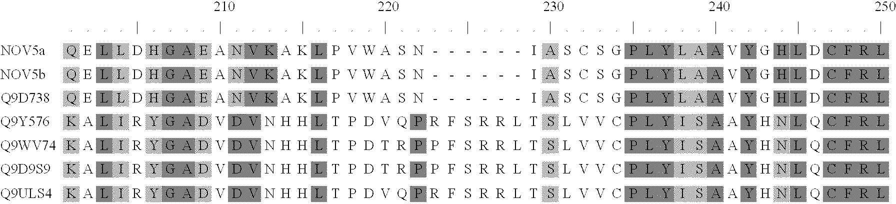

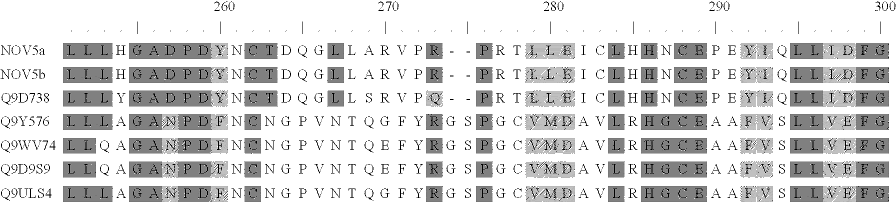

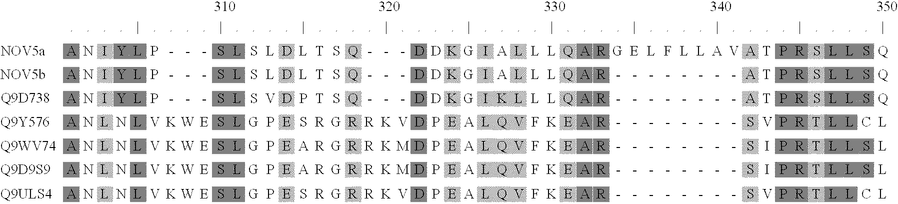

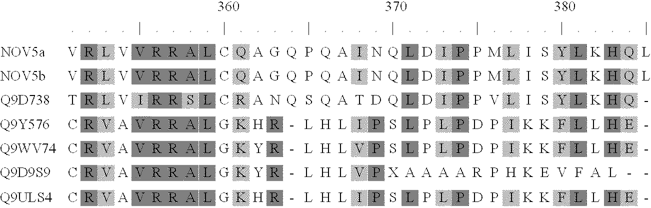

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 5H lists the domain description from DOMAIN analysis results against NOV5.

TABLE 5H
Domain Analysis of NOV5

| | Region of | Region of | | |
|---|---|---|---|---|
| | Homology to | Homology to | | |
| Model | NOV5a | NOV5b | Score (bits) | E value |
| Ank | 97-129 | 114-146 | 30.6 | 3.5e−05 |
| Ank | 130-162 | 147-179 | 26.4 | 6.9e−04 |
| Ank | 163-195 | 180-212 | 22.5 | 1e−02 |
| Ank | 205-237 | 222-254 | 24.7 | 2.1e−03 |
| Ank | 247-280 | 264-297 | 4.3 | 63 |

The presence of protein regions on NOV5 that are homologous to ankyrin repeat protein domains (Ank; IPR002110) is consistent with the identification of NOV5 as an ASB-1-like protein. This indicates that the NOV5 sequence has properties similar to those of other proteins known to contain these domains.

The above defined information for this invention suggests that NOV5 polypeptide is a member of a “ASB-1 family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in Bare lymphocyte syndrome, type II, glioma, nonsmall cell lung cancer, leukemia, and melanoma, pancreatic adenocarcinoma, dysplastic nevi, hereditary spherocytosis, elliptocytosis, pyropoikilocytosis , hemolytic anemia, Werner syndrome (scleroderma-like skin changes), and other pathologies and disorders. For example, a cDNA encoding the NOV5 may be useful in gene therapy, and the NOV5 may be useful when administered to a subject in need thereof. By way of nonlimiting example, the compositions of the present invention will have efficacy for treatment of patients suffering from Bare lymphocyte syndrome, type II, glioma, nonsmall cell lung cancer, leukemia, and melanoma, pancreatic adenocarcinoma, dysplastic nevi, hereditary spherocytosis, elliptocytosis, pyropoikilocytosis, hemolytic anemia, Werner syndrome (scleroderma-like skin changes). The novel nucleic acid encoding NOV5, and the NOV5 polypeptide of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding the NOV5 polypeptide of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV5 protein has multiple hydrophilic regions, each of which can be used as an immunogen. In particular, a region of strong hydrohilicity located at amino acids 60 to 125 may serve in this capacity.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

One or more consensus positions (Cons. Pos.) of the nucleotide sequence have been identified as SNPs as shown in Table 5I. “Depth” represents the number of clones covering the region of the SNP. The Putative Allele Frequency (Putative Allele Freq.) is the fraction of all the clones containing the SNP. A dash (“-”), when shown, means that a base is not present. The sign “>” means “is changed to”. The SNPs were detected using the minus strain.

A potential SNP is summarized in Table 51.

TABLE 5I
SNP Analysis of NOV5

| Consensus Positions | Depth | Change | Putative Allele Frequency |
|---|---|---|---|
| 653 | 38 | C > T | 0.447 |

NOV6

The disclosed novel NOV6 nucleic acid (SEQ ID NO:15) of 758 nucleotides (also referred to SC124881299_A) is shown in Table 6A. NOV6 encodes a novel neuronal tetraspanin-like protein and was identified on chromosome 11 by TblastN using CuraGen Corporation's sequence file for Neuronal Transpanin or homolog run against Genomic Daily Files made available by GenBank or from files download from the individual sequencing centers. The nucleic acid sequence was predicted from the genomic file GenBank accession number: AC016702.2 by homology to a known Neuronal Transpanin or homolog. An ORF begins with an ATG initiation codon at nucleotides 7-9 and ends with a TAG codon at nucleotides 754-56. A putative untranslated region upstream from the initiation codon and downstream from the termination codon is underlined in Table 6A, and the start and stop codons are in bold letters.

TABLE 6A
NOV6 Nucleotide Sequence (SEQ ID NO:15)

AGCACCATGGAAGGCGACTGTCTGAGCTGCATGAAGTATCTGATGTTTGTATTCAATTTCTTCATAT
TTCTGGGCGGGGCCTGCCTGCTGGCCATCGGCATCTGGGTCATGGTGGACCCCACCGGCTTCCGGGA
GATCGTGGCTGCCAATCCTCTGCTCCTCACGGGCGCCTACATCCTCCTGGCCATGGGGGGCCTGCTC
TTTCTGCTCGGCTTCCTGGGCTGCTGCGGGGCCGTCCGTGAGAACAAGTGTCTGCTGCTATTTTTCT
TCCTGTTCATCCTGATCATCTTCCTGGCAGAGCTCTCAGCAGCCATCCTGGCCTTCATCTTCAGGGA
AAATGTACTCACCCGAGAATTCTTCACCAAGCTCACCAAGCACTACCAGGGCAATAACGACACAGAC
GTCTTCTCTGCCACCTGGAACTCGGTCATGATCACATTTGGTTGCTGCGGGGTCAACGGGCCTGAAG
ACTTTAAGTTTGCATCTGTGTTTCGACTCCTGACCCTGGATAGTGAAGAGGTGCCGGAGCCTGCTGC
CTCGGGACGGGGTCAAAGTCGGGACGGGGTCCTGCTGAGCCGGGAGGAGTGCCTCCTGGGAAGGAGC
CTATTCCTAAACAAGCAGCAGGGCTGTTACACGGTGATCCTCAACACCTTCGAGACCTACGTCTACT
TGGCCGGAGCCCTTGCCATCGGGGTACTGGCCATCGAGCTTTTCGCCATGATCTTTGCCATGTGCCT
CTTCCGGGGCATCCAGTAGAG

The NOV6 protein (SEQ ID NO:16) encoded by SEQ ID NO:15 is 249 amino acid residues in length, has a molecular weight of 27588.3 Daltons, and is presented using the one-letter amino acid code in Table 6B. The Psort profile for NOV6 predicts that this sequence is likely to be localized in the plasma membrane with a certainty of 0.6400. Using Signal P analysis, it is predicted that the protein of the invention has a signal peptide and that the likely cleavage site for a NOV6 peptide is between positions 27 and 28, i.e., at the dash in the sequence ACL-LA.

TABLE 6B
Encoded NOV6 protein sequence (SEQ ID NO:16)

MEGDCLSCMKYLMFVFNFFIFLGGACLLAIGIWVMVDPTGFREIVAANPLLLTGAYILLAMGGLL
FLLGFLGCCGAVRENKCLLLFFFLFILIIFLAELSAAILAFIFRENVLTREFFTKLTKHYQGNND
TDVFSATWNSVMITFGCCGVNGPEDFKFASVFRLLTLDSEEVPEPAASGRGQSRDGVLLSREECL
LGRSLFLNKQQGCYTVILNTFETYVYLAGALAIGVLAIELFAMIFAMCLFRGIQ

A search against the Patp database, a proprietary database that contains sequences published in patents and patent publications, yielded several homologous proteins shown in Table 6C.

TABLE 6C
Patp results for NOV6

| | | | Smallest |
|---|---|---|---|
| | | | Sum |
| | Reading | High | Prob |
| Sequences producing High-scoring Segment Pairs: | Frame | Score | P(N) |
| >patp:AAB49503 Human protein clone HCE1K90 #1 | +1 | 1213 | 2.5e−123 |
| >patp:AAB93282 Human protein sequence SEQ ID NO: 12330 | +1 | 1143 | 2.5e−123 |
| >patp:AAH88457 Human protein clone P5EC0247 | +1 | 1144 | 5.1e−116 |
| >patp:AAB49509 Human protein clone ECE1E90 #2 | +1 | 833 | 4.6e−83 |

In a BLAST search of public sequence databases, it was found, for example, that the nucleic acid sequence has 582 of 755 bases (77%) identical to a Gallus gallus species, neuronal tetraspanin mRNA (GENBANK-ID: AF206661). The full amino acid sequence of the protein of the invention was found to have 196 of 249 amino acid residues (78%) identical to, and 214 of 249 residues (85%) positive with, the 247 amino acid residue NEURONAL TETRASPANIN-protein from Gallus gallus (ptnr:SPTREMBL-ACC: Q9PTE0). The global sequence homology (as defined by FASTA alignment with the full length sequence of this protein) is 82% amino acid homology and 79% amino acid identity. NOV6 also has homology to the proteins shown in the BLASTP data in Table 6D.

TABLE 6D
BLAST results for NOV6

| Gene Index/ | | Length | Identity | Positives | |
|---|---|---|---|---|---|
| Identifier | Protein/ Organism | (aa) | (%) | (%) | Expect |
| ptnr: TREMBLNEW- | CDNA FLJ14809 | 248 | 240/249 | 240/249 | 3.9e-123 |
| ACC: BAB55318 | FIS, CLONE | | (96%) | (96%) | |
| | NT2RP4001822, | | | | |
| | WEAKLY SIMILAR | | | | |
| | TO PLATELET- | | | | |
| | ENDOTHELIAL | | | | |
| | TETRASPAN | | | | |
| | ANTIGEN 3 | | | | |
| | [Homo sapiens] | | | | |
| ptnr: SPTREMBL- | NEURONAL | 247 | 196/249 | 214/249 | 8.9e-101 |
| ACC: Q9PTE0 | TETRASPANIN | | (78%) | (85%) | |
| | [Gallus gallus] | | | | |
| ptnr: SPTREMBL- | SIMILAR TO | 240 | 93/241 | 136/241 | 1.3e-35 |
| ACC: Q99J59 | TETRASPAN 1 | | (38%) | (56%) | |
| | [Mus musculus] | | | | |
| ptnr: SWISSNEW- | TETEASPANIN 1 | 241 | 85/245 | 130/245 | 3.7e-31 |
| ACC: O60635 | (TSPAN-1) | | (34%) | (53%) | |
| | (TETRASPAN NET-1) | | | | |
| | (TETRASPANIN TM4-C) | | | | |
| | [Homo sapiens] | | | | |

A multiple sequence alignment is given in Table 6E, with the NOV6 protein being shown on line 1 in Table 6E in a ClustalW analysis, and comparing the NOV6 protein with the related protein sequences shown in Table 6D. This BLASTP data is displayed graphically in the ClustalW in Table 6E.

TABLE 6E
ClustalW Analysis of NOV6

1) >NOV6; SEQ ID NO:15
2) >BAB55318/cDNA FLJ14809 FIS, Clone NT2RP4001822 weakly similar to platelet-endothelial
tetraspan antigen 3 [Homo sapiens]; SEQ ID NO:41
3) >Q9PTE0/Neuronal tetraspanin [Gallus gallus]; SEQ ID NO:41
4) >Q99J59/Similar to tetraspan 1 [Mus musculus]; SEQ ID NO:43
5) >O60635/Tetraspanin 1 (TSPAN-1) (TETRASPAN NET-1) (TETRASPANIN TM4-C) [Homo
sapiens]; SEQ ID NO:44

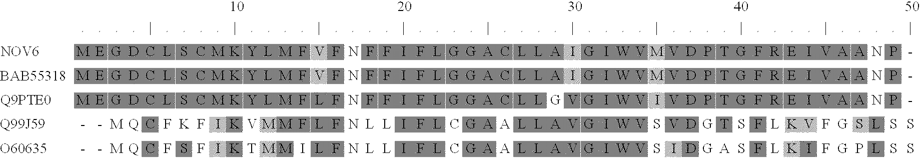

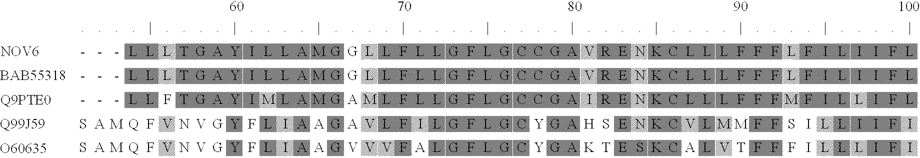

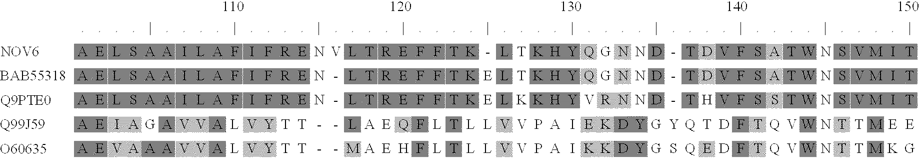

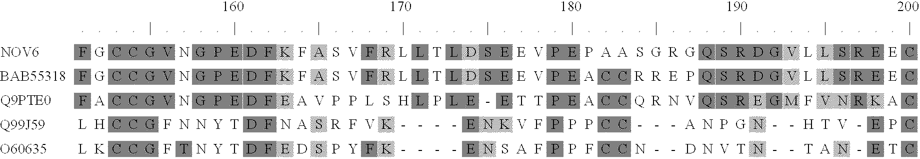

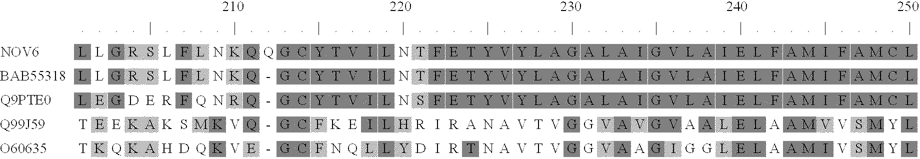

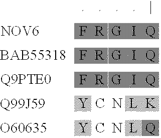

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 6F lists the domain description from DOMAIN analysis results against NOV6. This indicates that the NOV6 sequence has properties similar to those of other proteins known to contain these domains.

TABLE 6F
Domain Analysis of NOV6

| | Region of | | |
|---|---|---|---|
| Model | Homology | Score (bits) | E value |
| Transmembrane4 | 10-157 | 170.1 | 3.Ee−51 |
| Transmembrane4 | 205-254 | 47.4 | 1.5e−13 |

The presence of two NOV6 protein regions that are homologous to transmembrane 4 family domains (IPR000301) is consistent with the identification of NOV6 as a Neuronal Transpanin-like protein. This indicates that the NOV6 sequence has properties similar to those of other proteins known to contain these domains.

The above defined information for NOV6 suggests that NOV6 is a member of a “Transpanin family.” Therefore, the nucleic acids and proteins of the invention are useful in potential therapeutic applications implicated in various pathologies /disorders described and/or other pathologies/disorders. For example, a cDNA encoding the NOV6 may be useful in gene therapy, and the NOV6 may be useful when administered to a subject in need thereof. The nucleic acids and proteins of the invention are useful in potential therapeutic applications implicated in immune disorders, cancers, blood disorders, juvenile rheumatoid arthritis, Graves disease or immunocompromised disease, wound healing, X-linked mental retardation, fertility disorders, neurological disorders, and/or other pathologies and disorders. For example, a cDNA encoding the NOV6 may be useful in gene therapy, and the NOV6 may be useful when administered to a subject in need thereof. By way of nonlimiting example, the compositions of the present invention will have efficacy for treatment of patients suffering from immunedisorders, cancers, blood disorders, juvenile rheumatoid arthritis, Graves disease or immunocompromised disease, wound healing, X-linked mental retardation, fertility disorders, neurological disorders. The NOV6 nucleic acids, and the NOV6 polypeptide of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding the NOV6 of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen.

NOV7

The disclosed novel NOV7 nucleic acid (SEQ ID NO: 17) of 2390 nucleotides (also referred to SC_{—}18468704_A) is shown in Table 7A. NOV7 encodes a novel Semaphorin-like protein. An ORF begins with an ATG initiation codon at nucleotides 1-3 and ends with a TGA codon at nucleotides 2374-76. A putative untranslated region downstream from the termination codon is underlined in Table 7A, and the start and stop codons are in bold letters.

TABLE 7A
NOV7 Nucleotide Sequence (SEQ ID NO:17)

ATGGGAGGGGTGGCCCCAGGCTCTGAAGAGCGGCCATTCCTCAGATTCGAAGCTGAACACATCTCCA
ACTACACAGCCCTTCTGCTGAGCAGGGATGGCAGGACCCTGTACGTGGGTGCTCGAGAGGCCCTCTT
TGCACTCAGTAGCAACCTCAGCTTCCTGCCAGGCGGGGAGTACCAGGAGGTGCTTTGGGGTGCAGAC
GCAGAGAAGAAACAGCAGTGCAGCTTCAAGGGCAAGGACCCACAGCGCGACTGTCAAAACTACATCA
AGATCCTCCTGCCGCTCAGCGGCAGTCACCTGTTCACCTGTGGCACAGCAGCCTTCAGCCCCATGTG
TACCTACATCAACATGGAGAACTTCACCCTGGCAAGGGACGAGAAGGGGAACGCTTCTTCGGAAGAT
GGCAAGGGCCGTTGTCCCTTCGACCCGAATTTCAAGTCCACTGCCCTGGTGGTTGATGGCGAGCTCT
ACACTGGAACAGTCAGCAGCTTCCAAGGGAATGACCCGGCCATCTCGCGGAGCCAAAGCCTTCGCCC
CACCAAGACCGAGAGCTCCCTCAACTGGCTGCAAGACCCAGCTTTTGTGGCCTCAGCCTACATTCCT
GAGAGCCTGGGCAGCTTGCAAGGCGATGATGACAAGATCTACTTTTTCTTCAGCGAGACTGGCCAGG
AATTTGAGTTCTTTGAGAACACCATTGTGTCCCGCATTGCCCGCATCTGCAAGGGCGATGAGGGTGG
AGAGCGGGTGCTACAGCAGCGCTGGACCTCCTTCCTCAAGGCCCAGCTGCTGTGCTCACGGCCCGAC
GATGGCTTCCCCTTCAACGTGCTGCAGGATGTCTTCACGCTGAGCCCCAGCCCCCAGGACTGGCGTG
ACACCCTTTTCTATGGGGTCTTCACTTCCCAGTGGCACAGGGGAACTACAGAAGGCTCTGCCGTCTG
TGTCTTCACAATGAAGGATGTGCAGAGAGTCTTCAGCGGCCTCTACAAGGAGGTGAACCGTGAGACA
CAGCAGTGGTACACCGTGACCCACCCGGTGCCCACACCCCGGCCTGGAGCGTGCATCACCAACAGTG
CCCGGGAAAGGAAGATCAACTCATCCCTGCAGCTCCCAGACCGCGTGCTGAACTTCCTCAAGGACCA
CTTCCTGATGGACGGGCAGGTCCGAAGCCGCATGCTGCTGCTGCAGCCCCAGGCTCGCTACCAGCGC
GTGGCTGTACACCGCGTCCCTGGCCTGCACCACACCTACGATGTCCTCTTCCTGGGCACTGGTGACG
GCCGGCTCCACAAGGCAGTGAGCGTGGGCCCCCGGGTGCACATCATTGAGGAGCTGCAGATCTTCTC
ATCGGGACAGCCCGTGCAGAATCTGCTCCTGGACACCCACAGGGGGCTGCTGTATGCGGCCTCACAC
TCGGGCGTAGTCCAGGTGCCCATGGCCAACTGCAGCCTGTACAGGAGCTGTGGGGACTGCCTCCTCG
CCCGGAACCCCTACTGTGCTTGGAGCGGCTCCAGCTGCAAGCACGTCAACCTCTACCAGCCTCAGCT
GGCCACCAGGCCGTGGATCCAGGACATCGAGGGAGCCAGCGCCAAGGACCTTTGCAGCGCGTCTTCG
GTTGTGTCCCCGTCTTTTGTACCAACAGGGGAGAAACCATGTGAGCAAGTCCAGTTCCAGCCCAACA
CAGTGAACACTTTGGCCTGCCCGCTCCTCTCCAACCTGGCGACCCGACTCTGGCTACGCAACGGGGC
CCCCGTCAATGCCTCGGCCTCCTGCCACGTGCTACCCACTGGGGACCTGCTGCTGGTGGGCACCCAA
CAGCTGGGGGAGTTCCAGTGCTGGTCACTAGAGGAGGGCTTCCAGCAGCTGGTAGCCAGCTACTGCC
CAGAGGTGGTGGAGGACGGGGTGGCAGACCAAACAGATGAGGGTGGCAGTGTACCCGTCATTATCAG
CACATCGCGTGTGAGTGCACCAGCTGGTGGCAAGGCCAGCTGGGGTGCAGACAGGTCCTACTGGAAG
GAGTTCCTGGTGATGTGCACGCTCTTTGTGCTGGCCGTGCTGCTCCCAGTTTTATTCTTGCTCTACC
GGCACCGGAACAGCATGAAAGTCTTCCTGAAGCAGGGGGAATGTGCCAGCGTGCACCCCAAGACCTG
CCCTGTGGTGCTGCCCCCTGAGACCCGCCCACTCAACGGCCTAGGGCCCCCTAGCACCCCGCTCGAT
CACCGAGGGTACCAGTCCCTGTCAGACAGCCCCCCGGGTTCCCGAGTCTTCACTGAGTCAGAGAAGA
GGCCACTCAGCATCCAAGACAGCTTCGTGGAGGTATCCCCAGTGTGCCCCCGGCCCCGGGTCCGCCT
TGGTTCGGAGATCCGTGACTCTGTGGTGTGAGAGCTGACTTCCAG

The NOV7 protein (SEQ ID NO: 18) encoded by SEQ ID NO: 17 is 791 amino acid residues in length, has a molecular weight of 87484.2 Daltons, and is presented using the one-letter amino acid code in Table 7B. The Psort profile for NOV7 predicts that this sequence is likely to be localized in the plasma membrane with a certainty of 0.7000. Using Signal P analysis, it is predicted that the protein of the invention has a signal peptide and that the likely cleavage site for a NOV7 peptide is between positions 59 and 60, i.e., at the dash in the sequence GEY-QE.

TABLE 7B
Encoded NOV7 protein sequence (SEQ ID NO:18)

MGGVAPGSEERPFLRFEAEHISNYTALLLSRDGRTLYVGAREALFALSSNLSFLPGGEYQEVLWG
ADAEKKQQCSFKGKDPQRDCQNYIKILLPLSGSHLFTCGTAAFSPMCTYINMENFTLARDEKGNA
SSEDGKGRCPFDPNFKSTALVVDGELYTGTVSSFQGNDPAISRSQSLRPTKTESSLNWLQDPAFV
ASAYIPESLGSLQGDDDKIYFFFSETGQEFEFFENTIVSRIARICKGDEGGERVLQQRWTSFLKA
QLLCSRPDDGFPFNVLQDVFTLSPSPQDWRDTLFYGVFTSQWHRGTTEGSAVCVFTMKDVQRVFS
GLYKEVNRETQQWYTVTHPVPTPRPGACITNSARERKINSSLQLPDRVLNFLKDHFLMDGQVRSR
MLLLQPQARYQRVAVHRVPGLHHTYDVLFLGTGDGRLHKAVSVGPRVHIIEELQIFSSGQPVQNL
LLDTHRGLLYAASHSGVVQVPMANCSLYRSCGDCLLARNPYCAWSGSSCKHVNLYQPQLATRPWI
QDTEGASAKDLCSASSVVSPSFVPTGEKPCEQVQFQPNTVNTLACPLLSNLATRLWLRNGAPVNA
SASCHVLPTGDLLLVGTQQLGEFQCWSLEEGFQQLVASYCPEVVEDGVADQTDEGGSVPVIISTS
RVSAPAGGKASWGADRSYWKEFLVMCTLFVLAVLLPVLFLLYRHRNSMKVFLKQGECASVHPKTC
PVVLPPETRPLNGLGPPSTPLDHRGYQSLSDSPPGSRVFTESEKRPLSIQDSFVEVSPVCPRPRV
RLGSEIRDSVV

A search against the Patp database, a proprietary database that contains sequences published in patents and patent publications, yielded several homologous proteins shown in Table 7C.

TABLE 7C
Patp results for NOV7

| | | | Smallest |
|---|---|---|---|
| | | | Sum |
| | Reading | High | Prob |
| Sequences producing High-scoring Segment Pairs: | Frame | Score | P(N) |
| >patp:AAY99410 Human PRO1480 (UNQ749) | +1 | 4133 | 0.0 |
| >patp:AAB66159 Protein of the invention #71 | +1 | 4133 | 0.0 |
| >patp:AAB01396 Human Neuron-associated protein | +1 | 3592 | 0.0 |
| >patp:AAB41825 Human ORFX ORF1589 polypeptide | +1 | 1426 | 6.6e−146 |

In a search of sequence databases, it was found, for example, that the nucleic acid sequence has 1960 of 2362 bases (82%) identical to a Mus musculus Semaphorin mRNA (GENBANK-ID: X85992). The full amino acid sequence of the protein of the invention was found to have 660 of 783 amino acid residues (84%) identical to, and 716 of 783 residues (91%) positive with, the 782 amino acid residue protein Semaphorin from Mus musculus (ptnr:SPTREMBL-ACC: Q62179). The global sequence homology (as defined by FASTA alignment with the full length sequence of this protein) is 86% amino acid homology and 84% amino acid identity. NOV7 also has homology to the proteins shown in the BLASTP data in Table 7D.

TABLE 7D
BLAST results for NOV7

| Gene Index/ | | Length | Identity | Positives | |
|---|---|---|---|---|---|
| Identifier | Protein/Organism | (aa) | (%) | (%) | Expect |
| ptnr:SPTREMBL- | KIAA1745 PROTEIN | 893 | 286/311 | 286/311 | 0.0 |
| ACC:Q9C0B8 | [Homo sapiens] | | (91%) | (91%) | |
| ptnr:SWISSNEW- | SEMAPHORIN 4B | 673 | 667/672 | 669/672 | 0.0 |
| ACC:Q9NPR2 | [Homo sapiens] | | (99%) | (99%) | |
| ptnr:SWISSNEW- | SEMAPHORIN 4B | 782 | 660/783 | 716/783 | 0.0 |
| ACC:Q62179 | (SEMAPHORIN C) | | (84%) | (91%) | |
| | (SEMA C) | | | | |
| | [Mus musculus] | | | | |

A multiple sequence alignment is given in Table 7E, with the NOV7 protein being shown on line 1 in Table 7E in a ClustalW analysis, and comparing the NOV7 protein with the related protein sequences shown in Table 7D. This BLASTP data is displayed graphically in the ClustalW in Table 7E.

TABLE 7E
ClustalW Analysis of NOV7

1) >NOV7; SEQ ID NO:17
2) >Q9C0B8/KIAA1745 protein [Homo sapiens]; SEQ ID NO:45
3) >Q9NPR2/Semaphorin 4B [Homo sapiens]; SEQ ID NO:46
4) >Q62179/Semaphorin 4B (Semaphorin C) [Homo sapiens]; SEQ ID NO:47

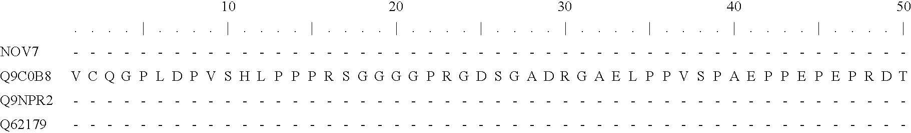

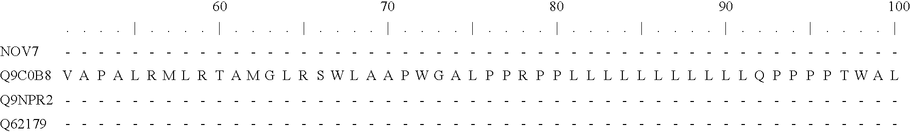

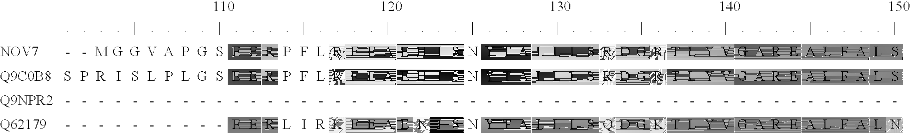

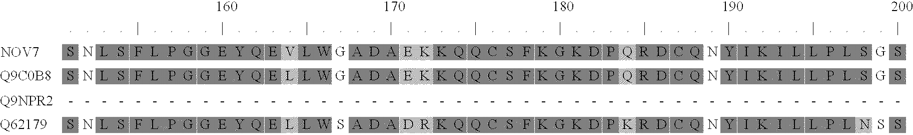

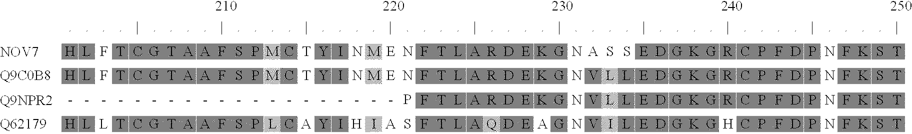

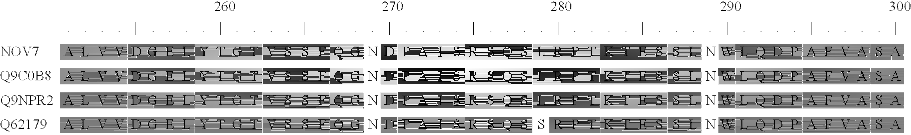

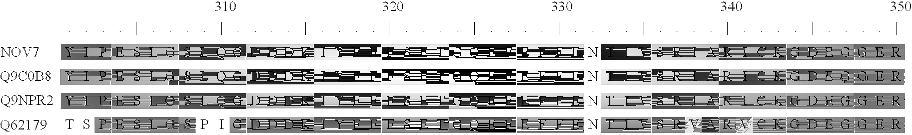

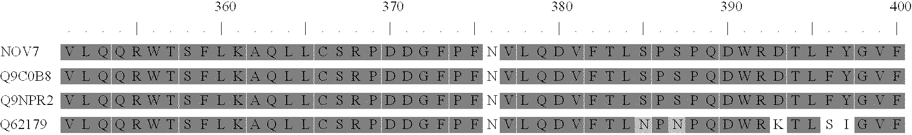

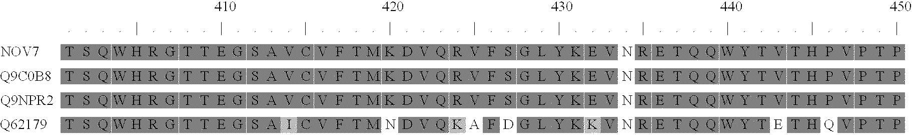

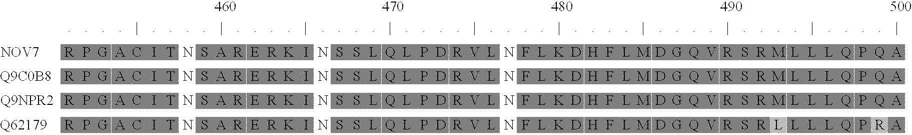

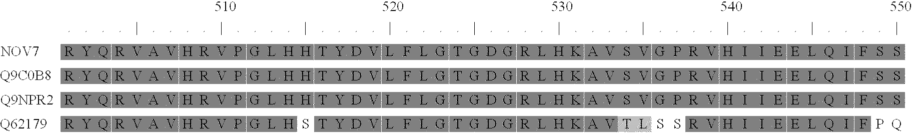

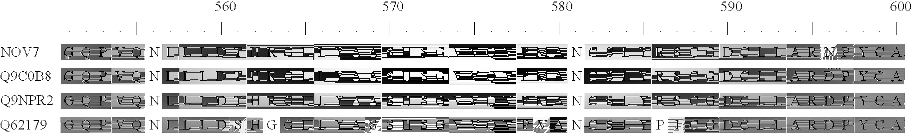

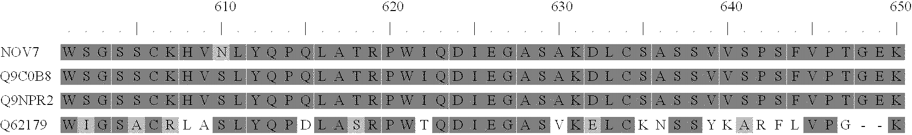

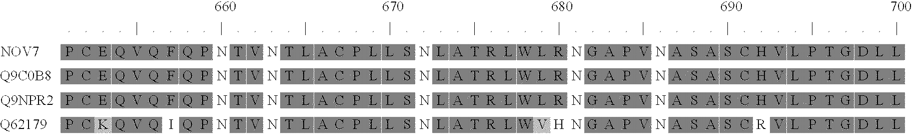

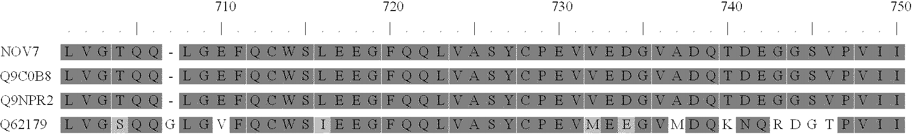

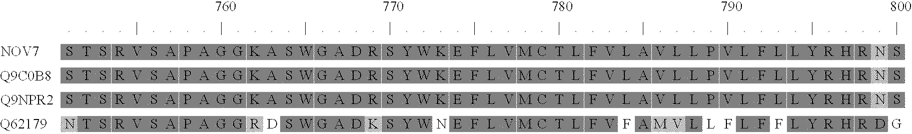

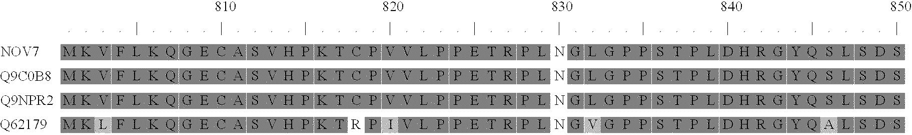

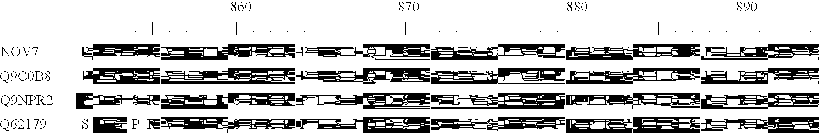

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 7F lists the domain description from DOMAIN analysis results against NOV7.

TABLE 7F
Domain Analysis of NOV7

| | Region of | | |
|---|---|---|---|
| Model | Homology | Score (bits) | E value |
| Sema | 24-461 | 545.9 | 2.8e−160 |
| Integrin B | 485-512 | 9.1 | 7.7e−03 |
| Plexin repeat | 479-533 | 18 | 3.4e−2 |

The presence of protein regions on NOV7 that are homologous to the the Sema domain (IPR001627) is consistent with identification of NOV7 as a Semaphorin-like protein. NOV7 also has protein regions similar to an integrin B domain and a Plexin repeat (IPR002165). This indicates that the NOV7 sequence has properties similar to those of other proteins known to contain these domains.

The above defined information for this invention suggests that a NOV7 is a semaphorin-like protein. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in Parkinson's disease, psychotic and neurological disorders, Alzheimers disease, cancer including but not limited to lung or breast cancer, endocrine disorders, inflammatory disorders, gastrointestinal disorders and disorders of the respiratory system and/or other pathologies and disorders. For example, a cDNA encoding the NOV7 pretein may be useful in gene therapy, and the NOV7 protein may be useful when administered to a subject in need thereof. By way of nonlimiting example, the compositions of the present invention will have efficacy for treatment of patients suffering from Parkinson's disease, psychotic and neurological disorders, Alzheimers disease, cancer including but not limited to lung or breast cancer, endocrine disorders, inflammatory disorders, gastro-intestinal disorders and disorders of the respiratory system. The novel nucleic acid encoding NOV7, and the NOV7 protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding the NOV7 of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

Table 8 provides a summary of the NOVX nucleic acids and their encoded polypeptides.

TABLE 8
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1 | AC068507A | 1 | 2 | NOPE-like protein |
| 2 | SC10176073_A | 3 | 4 | Cadherin-like protein |
| 3 | GMba380p16_A | 5 | 6 | Interferon-alpha-13-like protein |
| 4a | SC30236456_EXT1 | 7 | 8 | ADAM-like protein |
| 4b | AC024958_A | 9 | 10 | ADAM-like protein |
| 5a | SC_86058175_A | 11 | 12 | Ankyrin Repeat-containing (ASB- |
| | | | | 1)-like protein |
| 5b | CG7600-01 | 13 | 14 | Ankyrin Repeat-containing (ASB- |
| | | | | 1)-like protein |
| 6 | SC_124881299_A | 15 | 16 | Neuronal transpanin-like protein |
| 7 | SC_18468704_A | 17 | 18 | Semaphorin-like protein |

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17 or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, and 17, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, and 17, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, or 17 is one that is sufficiently complementary to the nucleotide sequence shown SEQ ID NOS: 1, 3, 5, 7, 9, 11, 13, 15, or 17 that it can hydrogen bond with little or no mismatches to the nucleotide sequence shown SEQ ID NOS: 1, 3, 5, 7, 9, 11, 13, 15, and 17, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, and 17, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, or 17; or an anti-sense strand nucleotide sequence of SEQ ID NOS: 1, 3, 5, 7, 9, 11, 13, 15, 17; or of a naturally occurring mutant of SEQ ID NOS: 1, 3, 5, 7, 9, 11, 13, 15, and 17.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, or 17 that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, and 17 due to degeneracy of the genetic code and thus encode the same NOVX proteins as that encoded by the nucleotide sequences shown in SEQ ID NOS: 1, 3, 5, 7, 9,11, 13, 15, and 17. In another embodiment, an isolated nucleic acid molecule of the invention has a nucleotide sequence encoding a protein having an amino acid sequence shown in SEQ ID NOS: 2,4, 6, 8, 10, 12, 14, 16, and 18.

In addition to the human NOVX nucleotide sequences shown in SEQ ID NOS: 1, 3, 5, 7, 9, 11, 13, 15, and 17, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, and 17 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, and 17. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2X SSC, 0.01% BSA at 50° C. An isolated nucleic acid molecule of the invention that hybridizes under stringent conditions to the sequences SEQ ID NOS: 1, 3, 5, 7, 9, 11, 13, 15, and 17, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, and 17, or fragments, analogs or derivatives thereof, under conditions of moderate stringency is provided. A non-limiting example of moderate stringency hybridization conditions are hybridization in 6X SSC, 5X Denhardt's solution, 0.5% SDS and 100 mg/ml denatured salmon sperm DNA at 55° C., followed by one or more washes in 1X SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, N.Y.

In a third embodiment, a nucleic acid that is hybridizable to the nucleic acid molecule comprising the nucleotide sequences SEQ ID NOS: 1, 3, 5, 7, 9, 11, 13, 15, and 17, or fragments, analogs or derivatives thereof, under conditions of low stringency, is provided. A non-limiting example of low stringency hybridization conditions are hybridization in 35% formamide, 5X SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2X SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, and 17, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, and 18. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 2, 4, 6, 8, 10, 12, 14, 16, or 18 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, or 18. Preferably, the protein encoded by the nucleic acid molecule is at least about 60% homologous to SEQ ID NOS: 2, 4, 6, 8, 10, 12, 14, 16, or 18; more preferably at least about 70% homologous SEQ ID NOS: 2, 4, 6, 8, 10, 12, 14, 16, and 18; still more preferably at least about 80% homologous to SEQ ID NOS: 2 , 4 , 6 , 8, 10, 12, 14, 16, and 18; even more preferably at least about 90% homologous to SEQ ID NOS: 2, 4, 6, 8, 10, 12, 14, 16, and 18; and most preferably at least about 95% homologous to SEQ ID NOS: 2, 4, 6, 8, 10, 12, 14, 16, and 18.

An isolated nucleic acid molecule encoding an NOVX protein homologous to the protein of SEQ ID NOS: 2, 4, 6, 8, 10, 12, 14, 16, and 18 can be created by introducing one or more nucleotide substitutions, additions or deletions into the nucleotide sequence of SEQ ID NOS: 1, 3, 5, 7, 9, 11, 13, 15, and 17, such that one or more amino acid substitutions, additions or deletions are introduced into the encoded protein.

Mutations can be introduced into SEQ ID NOS: 2, 4, 6, 8, 10, 12, 14, 16, and 18 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, and 17, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, and 17, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, and 18, or antisense nucleic acids complementary to an NOVX nucleic acid sequence of SEQ ID NOS: 1, 3, 5, 7, 9, 11, 13, 15, and 17, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, and 17). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996.supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, and 18. The invention also includes a mutant or variant protein any of whose residues may be changed from the corresponding residues shown in SEQ ID NOS: 2, 4, 6, 8, 10, 12, 14, 16, and 18 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, and 18) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, and 18. In other embodiments, the NOVX protein is substantially homologous to SEQ ID NOS: 2, 4, 6, 8, 10, 12, 14, 16, and 18, and retains the functional activity of the protein of SEQ ID NOS: 2, 4, 6, 8, 10, 12, 14, 16, and 18, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, and 18, and retains the functional activity of the NOVX proteins of SEQ ID NOS: 2, 4, 6, 8, 10, 12, 14, 16, and 18.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, and 17.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, and 18), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nuci. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS: 2, 4, 6, 8, 10, 12, 14, 16, and 18 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J Mol. Biol. 157: 105-142, each incorporated herein by reference in their entirety).

As disclosed herein, NOVX protein sequences of SEQ ID NOS: 2, 4, 6, 8, 10, 12, 14, 16, 18, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F_{(ab′)2} fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS: 2, 4, 6, 8, 10, 12, 14, 16, 18, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: Monoclonal Antibodies and Cancer Therapy, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: Monoclonal Antibodies and Cancer Therapy, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufmnan, et al., 1987. EMBO J 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryoric and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, and 17 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, and 17), but more preferably, is a non-human homologue of a human NOVX gene. For example, a mouse homologue of human NOVX gene of SEQ ID NOS: 1, 3, 5, 7, 9, 11, 13, 15, and 17 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for an example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Patent No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g., developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease(possesses anti-microbial activity). In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997.,Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Tritone® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, and 17, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, and 17 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease (possesses anti-microbial activity). The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, and 17, or aportion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Nat. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders [the disorders include developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease (possesses anti-microbial activity)]. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include endocrine disorders; developmental disorders; gastrointestinal diseases; lung diseases; respiratory disorders; vascular diseases; blood disorders; autoimmune and immune disorders; multiple sclerosis; inflammatory disorders and Hepatitis C; Trauma; regeneration (in vitro and in vivo); viral/bacterial/parasitic infections; hyperthyroidism; hypothyroidism; endometriosis; fertility; angiogenesis; hypertension; stroke; ischemia; arteriosclerosis; aneurysms; stroke; and bleeding disorders; Bare lymphocytic syndrome; type II; hereditary spherocytosis; elliptocytosis; pyropoikilocytosis; hemolytic anemia; Werner syndrome (scleroderma-like skin changes); juvenile rheumatoid arthritis; Graves disease; wound healing; X-linked mental retardation; and fertility disorders; psychotic and neurological disorders; neuronal degeneration; including but not limited to Parkinson's and Alzheimer's Disease; dysplastic nevi and cancer; including but not limited to; glioma; leukemia; melanoma; pancreatic adenocarcinoma; non-Hodgkin's lymphoma; renal cancer; hepatocellular carcinomas; and myeloid leukemia lung or breast cancer, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: developmental disorders, endocrine disorders, vascular disorders, infectious disease, anorexia, cancer, neurodegenerative disorders, lung disorders, reproductive disorders, Alzheimer's Disease, Parkinson's disease, immune and autoimmune disorders, and hematopoietic disorders, or other disorders related to cell signal processing and metabolic pathway modulation.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: developmental disorders, endocrine disorders, vascular disorders, infectious disease, anorexia, cancer, neurodegenerative disorders, lung disorders, reproductive disorders, Alzheimer's Disease, Parkinson's Disease, immune and autoimmune disorders, and hematopoietic disorders, or other disorders related to cell signal processing and metabolic pathway modulation.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLE 1

### Quantitative expression analysis of clones in various cells and tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to constitutively expressed genes such as b-actin and GAPDH. RNA (˜50 ng total or ˜1 ng polyA+) was converted to cDNA using the TAQMAN( Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 480C. cDNA (5 ul) was then transferred to a separate plate for the TAQMAN® reaction using b-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 500C; 10 min. at 950C; 15 sec. at 950C/1 min. at 600C (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their b-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version 1 for Apple Computer's Macintosh Power PC) or a similar algorithm using the target sequence as input. Default settings were used for reaction conditions and the following parameters were set before selecting primers: primer concentration=250 nM, primer melting temperature (Tm) range=58-60° C., primer optimal Tm=59° C., maximum primer difference=2° C., probe does not have 5′ G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1X TaqManÔ PCR Master Mix for the PE Biosystems 7700, with 5 mM MgC12, dNTPs (dA, G, C, U at 1:1:1:2 ratios), 0.25 U/ml AmpliTaq GoldÔ(PE Biosystems), and 0.4 U/ml RNase inhibitor, and 0.25 U/ml reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl efflusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 mM non essential amino acids (Gibco/Life Technologies, Rockville, Md.), 1 mM sodium pyruvate (Gibco), mercaptoethanol 5.5×10-5 M (Gibco), and 10 mM Hepes (Gibco) and Interleukin 2 for 4-6 days. Cells were then either activated with 10-20 ng/ml PMA and 1-2 mg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco) with PHA (phytohemagglutinin) or PWM (pokeweed mitogen) at approximately 5 mg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×106 cells/ml in DMEM 5% FCS (Hyclone), 100 mM non essential amino acids (Gibco), 1 mM sodium pyruvate (Gibco), mercaptoethanol (5.5×10-5 M) (Gibco), and 10 mM Hepes (Gibco). The MLR was cultured and samples taken at various time points ranging from 1- 7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 mM non essential amino acids (Gibco), 1 mM sodium pyruvate (Gibco), mercaptoethanol 5.5×10-5 M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 mM non essential amino acids (Gibco), 1 mM sodium pyruvate (Gibco), mercaptoethanol 5.5×10-5 M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 mg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CDl9 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco) and plated at 106 cells/ml onto Falcon 6 well tissue culture plates that had been coated overnight with 0.5 mg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 106 cells/ml in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco). To activate the cells, we used PWM at 5 mg/ml or anti-CD40 (Pharmingen) at approximately 10 mg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 ,ug/ml OKT3 (ATCC), and then washed twice with PBS. Umbilical cord blood CD4 lymphocytes (Poietic Systems, German Town, Md.) were cultured at 105-106 cells/ml in DMEM 5% FCS (Hyclone), 100 mM non essential amino acids (Gibco), 1 mM sodium pyruvate (Gibco), mercaptoethanol 5.5×10-5 M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 mM non essential amino acids (Gibco), 1 mM sodium pyruvate (Gibco), mercaptoethanol 5.5×10-5 M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×105 cells/ml for 8 days, changing the media every 3 days and adjusting the cell concentration to 5×105 cells/ml. For the culture of these cells, we used DMEM or RPMI (as recommended by the ATCC), with the addition of 5% FCS (Hyclone), 100 mM non essential amino acids (Gibco), 1 mM sodium pyruvate (Gibco), mercaptoethanol 5.5×10-5 M (Gibco), 10 mM Hepes (Gibco). RNA was either prepared from resting cells or cells activated with PMA at 10 ng/ml and ionomycin at 1 mg/ml for 6 and 14 hours. Keratinocyte line CCD106 and an airway epithelial tumor line NCI-H292 were also obtained from the ATCC. Both were cultured in DMEM 5% FCS (Hyclone), 100 mM non essential amino acids (Gibco), 1 mM sodium pyruvate (Gibco), mercaptoethanol 5.5×10-5 M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 107 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 ml of RNAse-free water and 35 ml buffer (Promega) 5 ml DTT, 7 ml RNAsin and 8 ml DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

### EXAMPLE 2

### Quantitative expression analysis of NOV1 expression in various cells and tissues

Expression of gene AC068507A was assessed using the primer-probe set Ag902, described in Table AA. Results of the RTQ-PCR runs are shown in Tables AB, AC, AD, AE.

Table 9. Probe Name Ag902

| | | | | Start |
|---|---|---|---|---|
| Primers | Sequences | TM | Length | Position |
| Forward | 5′-ATCAAACAGCTCCACATCCAT-3′ | 59.4 | 21 | 2277 |
| | SEQ ID NO: 48 | | | |
| Probe | AAAAGCCAGATTTCACCACAGTCAAG- | 65.7 | 26 | 2312 |
| | 3′-TAMRA | | | |
| | SEQ ID NO: 49 | | | |
| Reverse | 5′-AGCGCACAGTGTAGTTGACAAT-3′ | 59.9 | 22 | 2338 |
| | SEQ ID NO: 50 | | | |

TABLE 10
Panel 1.2

| | Relative Expression (%) | |
|---|---|---|
| | 1.2tm1045f_{—} | 1.2tm1325f_{—} |
| Tissue Name | ag902 | ag902 |
| Endothelial cells | 0.0 | 0.0 |
| Fetal heart | 2.9 | 12.0 |
| Pancreas | 5.1 | 3.8 |
| Pancreatic ca. CAPAN 2 | 0.0 | 20.3 |
| Adrenal Gland (new lot*) | 20.7 | 55.9 |
| Thyroid | 15.4 | 14.4 |
| Salavary gland | 2.4 | 5.5 |
| Pituitary gland | 14.5 | 11.7 |
| Brain (fetal) | 27.9 | 26.4 |
| Brain (whole) | 6.0 | 8.5 |
| Brain (amygdala) | 5.1 | 7.9 |
| Brain (cerebellum) | 2.0 | 8.7 |
| Brain (bippocampus) | 6.1 | 10.0 |
| Brain (thalamus) | 5.6 | 8.9 |
| Cerebral Cortex | 7.2 | 23.5 |
| Spinal cord | 6.7 | 15.2 |
| CNS ca. (glio/astro) U87-MG | 2.3 | 2.7 |
| CNS ca. (glio/astro) U-118-MG | 3.3 | 4.8 |
| CNS ca. (astro) SW1783 | 0.0 | 0.0 |
| CNS ca.* (neuro; met ) SK-N-AS | 7.2 | 7.7 |
| CNS ca. (astro) SF-539 | 5.1 | 5.9 |
| CNS ca. (astro) SNB-75 | 0.1 | 1.1 |
| CNS ca. (glio) SNB-19 | 0.7 | 2.6 |
| CNS ca. (glio) U251 | 4.3 | 11.7 |
| CNS ca. (glio) SF-295 | 0.9 | 1.0 |
| Heart | 7.2 | 10.0 |
| Skeletal Muscle (new lot*) | 100.0 | 100.0 |
| Bone marrow | 0.0 | 0.3 |
| Thymus | 1.3 | 2.7 |
| Spleen | 0.2 | 0.4 |
| Lymph node | 2.4 | 5.2 |
| Colorectal | 0.3 | 1.6 |
| Stomach | 6.2 | 14.2 |
| Small intestine | 5.9 | 7.3 |
| Colon ca. SW480 | 0.4 | 0.6 |
| Colon ca.* (SW480 met)SW620 | 1.6 | 1.9 |
| Colon ca. HT29 | 0.0 | 0.0 |
| Colon ca. HCT-116 | 0.2 | 0.2 |
| Colon ca. CaCo-2 | 3.2 | 4.1 |
| 83219 CC Well to Mod Diff | 0.5 | 1.3 |
| (ODO3866) | | |
| Colon ca. HCC-2998 | 0.1 | 0.3 |
| Gastric ca.* (liver met) NCI-N87 | 0.0 | 0.3 |
| Bladder | 3.5 | 4.9 |
| Trachea | 1.1 | 3.0 |
| Kidney | 0.2 | 0.8 |
| Kidney (fetal) | 7.7 | 18.8 |
| Renal ca. 786-0 | 0.0 | 0.0 |
| Renal ca. A498 | 0.4 | 0.9 |
| Renal ca. RXF 393 | 5.0 | 15.1 |
| Renal ca. ACEN | 1.8 | 5.4 |
| Renal ca. UO-31 | 8.6 | 13.3 |
| Renal ca. TK-10 | 8.4 | 13.4 |
| Liver | 0.4 | 0.8 |
| Liver (fetal) | 4.4 | 6.2 |
| Liver ca. (hepatoblast) HepG2 | 0.0 | 0.2 |
| Lung | 0.4 | 0.5 |
| Lung (fetal) | 13.4 | 12.2 |
| Lung ca. (small cell) LX-1 | 1.5 | 3.3 |
| Lung ca. (small cell) NCI-H69 | 0.4 | 0.3 |
| Lung ca. (s.cell var.) SEP-77 | 0.2 | 0.0 |
| Lung ca. (large cell)NCI-H460 | 0.4 | 1.7 |
| Lung ca. (non-sm. cell) A549 | 2.8 | 5.0 |
| Lung ca. (non-s.cell) NCI-H23 | 1.6 | 2.7 |
| Lung ca (non-s.cell) HOP-62 | 2.7 | 4.5 |
| Lung ca. (non-s.d) NCI-H522 | 33.9 | 50.3 |
| Lung ca. (squam.) SW 900 | 2.2 | 4.3 |
| Lung ca. (squam.) NCI-H596 | 0.2 | 0.2 |
| Mammary gland | 7.3 | 7.7 |
| Breast ca.* (pl. effusion) MCF-7 | 0.0 | 0.0 |
| Breast ca.* (pl.ef) MDA-MB-231 | 1.1 | 2.3 |
| Breast ca.* (pl. effusion) T47D | 0.6 | 0.7 |
| Breast ca. BT-549 | 0.0 | 0.0 |
| Breast ca. MDA-N | 0.0 | 0.0 |
| Ovary | 9.0 | 33.2 |
| Ovarian Ca. OVCAR-3 | 0.5 | 0.8 |
| Ovarian Ca. OVCAR-4 | 1.1 | 1.7 |
| Ovarian Ca. OVCAR-5 | 4.4 | 3.8 |
| Ovarian Ca. OVCAR-8 | 4.6 | 14.1 |
| Ovarian Ca. IGROV-l | 0.4 | 0.5 |
| Ovarian Ca.* (asCites) SK-OV-3 | 1.1 | 2.1 |
| Uterus | 3.2 | 5.4 |
| Placenta | 36.9 | 50.0 |
| Prostate | 1.2 | 2.5 |
| Prostate ca.* (bone met)PC-3 | 1.6 | 3.8 |
| Testis | 4.1 | 5.1 |
| Melanoma Hs688CA) .T | 1.7 | 2.0 |
| Melanoma* (met) Hs688CB).T | 2.2 | 2.7 |
| Melanoma UACC-62 | 0.3 | 1.0 |
| Melanoma M14 | 0.0 | 0.0 |
| Melanoma LOX IMVI | 0.0 | 0.0 |
| Melanoma* (met) SK-MEL-5 | 0.2 | 0.9 |
| Adipose | 0.8 | 9.1 |

TABLE 11
Panel 1.2

| | Relative Expression (%) | |
|---|---|---|
| | 1.2tm1045f_{—} | 1.2tm1325f_{—} |
| Tissue Name | ag902 | ag902 |
| Endothelial cells | 0.0 | 0.0 |
| Fetal heart | 2.9 | 12.0 |
| Pancreas | 5.1 | 3.8 |
| Pancreatic Ca. CAPAN 2 | 0.0 | 20.3 |
| Adrenal Gland (new lot*) | 20.7 | 55.9 |
| Thyroid | 15.4 | 14.4 |
| Salavary gland | 2.4 | 5.5 |
| Pituitary gland | 14.5 | 11.7 |
| Brain (fetal) | 27.9 | 26.4 |
| Brain (whole) | 6.0 | 8.5 |
| Brain (amygdala) | 5.1 | 7.9 |
| Brain (cerebellum) | 2.0 | 8.7 |
| Brain (hippocampus) | 6.1 | 10.0 |
| Brain (thalamus) | 5.6 | 8.9 |
| Cerebral Cortex | 7.2 | 23.5 |
| Spinal cord | 6.7 | 15.2 |
| CNS ca. (glio/astro) U87-MG | 2.3 | 2.7 |
| CNS ca. (glio/astro) U-118--MG | 3.3 | 4.8 |
| CNS ca. (astro) SW1783 | 0.0 | 0.0 |
| CNS ca.* (neuro; met ) 9K-N-AS | 7.2 | 7.7 |
| CNS ca. (astro) SF-539 | 5.1 | 5.9 |
| CNS ca. (astro) SNB-75 | 0.1 | 1.1 |
| CNS ca. (qua) SNB-19 | 0.7 | 2.6 |
| CNS ca. (quo) U251 | 4.3 | 11.7 |
| CNS ca. (glio) SF-295 | 0.9 | 1.0 |
| Heart | 7.2 | 10.0 |
| Skeletal Muscle (new lot*) | 100.0 | 100.0 |
| Bone marrow | 0.0 | 0.3 |
| Thymus | 1.3 | 2.7 |
| Spleen | 0.2 | 0.4 |
| Lymph node | 2.4 | 5.2 |
| Colorectal | 0.3 | 1.6 |
| Stomach | 6.2 | 14.2 |
| Small intestine | 5.9 | 7.3 |
| Colon ca. SW480 | 0.4 | 0.6 |
| Colon ca.* (SW480 met)SW620 | 1.6 | 1.9 |
| Colon ca. HT29 | 0.0 | 0.0 |
| Colon ca. HCT-116 | 0.2 | 0.2 |
| Colon ca. CaCo-2 | 3.2 | 4.1 |
| 83219 CC Well to Mod Diff | 0.5 | 1.3 |
| (ODO3866) | | |
| Colon ca. HCC-2998 | 0.1 | 0.3 |
| Gastric ca.* (liver met) NCI-N87 | 0.0 | 0.3 |
| Bladder | 3.5 | 4.9 |
| Trachea | 1.1 | 3.0 |
| Kidney | 0.2 | 0.8 |
| Kidney (fetal) | 7.7 | 18.8 |
| Renal ca. 786-0 | 0.0 | 0.0 |
| Renal ca. A498 | 0.4 | 0.9 |
| Renal ca. RXF 393 | 5.0 | 15.1 |
| Renal Ca. ACHN | 1.8 | 5.4 |
| Renal Ca. UO-31 | 8.6 | 13.3 |
| Renal Ca. TK-l0 | 8.4 | 13.4 |
| Liver | 0.4 | 0.8 |
| Layer (fetal) | 4.4 | 6.2 |
| Liver Ca. (hepatoblast) HepG2 | 0.0 | 0.2 |
| Lung | 0.4 | 0.5 |
| Lung (fetal) | 13.4 | 12.2 |
| Lung ca. (small cell) LX-1 | 1.5 | 3.3 |
| Lung ca. (small cell) NCI-H69 | 0.4 | 0.3 |
| Lung ca. (s.cell var.) SHP-77 | 0.2 | 0.0 |
| Lung ca. (large cell)NCI-H460 | 0.4 | 1.7 |
| Lung ca. (non-sm. cell) A549 | 2.8 | 5.0 |
| Lung ca. (non-s.cell) NCI-H23 | 1.6 | 2.7 |
| Lung ca (non-s.cell) HOP-62 | 2.7 | 4.5 |
| Lung ca. (non-s.d) NCI-H522 | 33.9 | 50.3 |
| Lung ca. (squam.) SW 900 | 2.2 | 4.3 |
| Lung ca. (squam.) NCI-H596 | 0.2 | 0.2 |
| Mammary gland | 7.3 | 7.7 |
| Breast ca.* (pl. effusion) MCF-7 | 0.0 | 0.0 |
| Breast ca.* (pl.ef) MDA-MB-231 | 1.1 | 2.3 |
| Breast ca.* (pl. effusion) T47D | 0.6 | 0.7 |
| Melanoma* (met) Hs68B(B).T | 2.2 | 2.7 |
| Melanoma UACC-62 | 0.3 | 1.0 |
| Melanoma M14 | 0.0 | 0.0 |
| Melanoma LOX IMVI | 0.0 | 0.0 |
| Melanoma* (met) SK-MEL-5 | 0.2 | 0.9 |
| Adipose | 0.8 | 9.1 |

TABLE 12
Panel 2D

| | Relative Expression (%) | |
|---|---|---|
| | 2Dtm2526f_{—} | 2Dtm3155f_{—} |
| Tissue Name | ag902 | ag902 |
| Normal Colon GENPAK 061003 | 24.1 | 28.9 |
| 83219 CC Well to Mod Diff | 4.1 | 5.0 |
| (ODO3866) | | |
| 83220 CC NAT (ODO3866) | 5.9 | 3.6 |
| 83221 CC Gr.2 rectosigmoid | 4.6 | 4.0 |
| (ODO3868) | | |
| 83222 CC NAT (ODO3868) | 1.3 | 1.2 |
| 83235 CC Mod Diff (ODO3920) | 19.6 | 19.5 |
| 83236 CC NAT (ODO3920) | 6.2 | 9.3 |
| 83237 CC Gr.2 ascend colon | 25.9 | 34.2 |
| (ODO3921) | | |
| 83238 CC NAT (ODO3921) | 6.7 | 7.2 |
| 83241 CC from Partial Hepatectomy | 3.3 | 2.6 |
| (ODO4309) | | |
| 83242 Liver NAT (ODO4309) | 0.8 | 0.6 |
| 87472 Colon mets to lung | 5.0 | 3.2 |
| (OD04451-01) | | |
| 87473 Lung NAT (OD04451-02) | 0.6 | 0.0 |
| Normal Prostate Clontech A+ 6546-1 | 4.8 | 3.0 |
| 84140 Prostate Cancer (ODO4410) | 3.5 | 3.9 |
| 84141 Prostate NAT (ODO4410) | 6.0 | 6.8 |
| 87073 Prostate Cancer (ODO4720-01) | 0.9 | 1.2 |
| 87074 Prostate NAT (ODO4720-02) | 6.3 | 2.4 |
| Normal Lung GENPAK 061010 | 16.5 | 18.6 |
| 83239 Lung Met to Muscle (ODO4286) | 31.4 | 28.1 |
| 83240 Muscle NAT (ODO4286) | 53.6 | 66.4 |
| 84136 Lung Malignant Cancer | 8.7 | 10.0 |
| (ODO3126) | | |
| 84137 Lung NAT (ODO3126) | 8.8 | 4.6 |
| 84871 Lung Cancer (ODO4404) | 7.2 | 9.2 |
| 84872 Lung NAT (ODO4404) | 6.0 | 7.9 |
| 84875 Lung Cancer (OD04565) | 4.6 | 4.3 |
| 84876 Lung NAT (ODO4565) | 1.7 | 0.9 |
| 85950 Lung Cancer (ODO4237-01) | 22.4 | 16.8 |
| 85970 Lung NAT (ODO4237-02) | 1.5 | 1.9 |
| 83255 Ocular Mel Met to | 1.4 | 1.4 |
| Liver (ODO4310) | | |
| 83256 Liver NAT (ODO4310) | 0.1 | 0.2 |
| 84139 Melanoma Mets to | 0.3 | 0.6 |
| Lung (ODO4321) | | |
| 84138 Lung NAT (ODO4321) | 1.2 | 2.1 |
| Normal Kidney GENPAK 061008 | 3.6 | 4.7 |
| 83786 Kidney Ca, Nuclear | 78.5 | 72.2 |
| grade 2 (ODO4338) | | |
| 83787 Kidney NAT (ODO4338) | 4.2 | 6.0 |
| 83788 Kidney Ca Nuclear | 62.0 | 54.3 |
| grade 1/2 (ODO4339) | | |
| 83789 Kidney NAT (ODO4339) | 3.0 | 2.9 |
| 83790 Kidney Ca, Clear cell | 0.5 | 1.2 |
| type (ODO4340) | | |
| 83791 Kidney NAT (ODO4340) | 3.5 | 3.7 |
| 83792 Kidney Ca, Nuclear | 7.5 | 9.5 |
| grade 3 (ODO4348) | | |
| 83793 Kidney NAT (ODO4348) | 3.1 | 4.2 |
| 87474 Kidney Cancer CODO4622-01) | 100.0 | 100.0 |
| 87475 Kidney NAT (ODO4622-03) | 0.0 | 0.6 |
| 85973 Kidney Cancer (ODO4450-01) | 11.2 | 12.7 |
| 85974 Kidney NAT (ODO4450-03) | 2.0 | 3.2 |
| Kidney Cancer Clontech 8120607 | 7.1 | 6.7 |
| Kidney NAT Clontech 8120608 | 0.5 | 0.8 |
| Kidney Cancer Clontech 8120613 | 1.6 | 0.9 |
| Kidney NAT Clontech 8120614 | 1.5 | 1.2 |
| Kidney Cancer Clontech 9010320 | 90.8 | 88.3 |
| Kidney NAT Clontech 9010321 | 5.1 | 4.1 |
| Normal Uterus GENPAK 061018 | 9.5 | 6.7 |
| Uterus Cancer GENPAK 064011 | 20.9 | 18.9 |
| Normal Thyroid Clontech A+ 6570-1 | 42.9 | 41.2 |
| Thyroid Cancer GENPAK 064010 | 11.8 | 16.6 |
| Thyroid Cancer INVITROGEN | 12.8 | 9.9 |
| A302152 | | |
| Thyroid NAT INVITROGEN A302153 | 9.5 | 14.4 |
| Normal Breast GENPAK 061019 | 16.5 | 18.9 |
| 84877 Breast Cancer (ODO4566) | 2.5 | 4.0 |
| 85975 Breast Cancer (ODO4590-01) | 5.7 | 3.1 |
| 85976 Breast Cancer Mets | 8.2 | 6.9 |
| (ODO4590-03) | | |
| 87070 Breast Cancer | 5.4 | 3.4 |
| Metastasis (ODO4655-05) | | |
| GENPAK Breast Cancer 064006 | 11.3 | 8.4 |
| Breast Cancer Res. Gen. 1024 | 28.7 | 34.4 |
| Breast Cancer Clontech 9100266 | 11.1 | 12.2 |
| Breast NAT Clontech 9100265 | 21.9 | 27.9 |
| Breast Cancer TNVTTROGEN | 27.2 | 19.5 |
| A209073 | | |
| Breast NAT INVITROGEN A2090734 | 20.4 | 17.1 |
| Normal Liver GENPAK 061009 | 0.4 | 0.3 |
| Liver Cancer GENPAK 064003 | 0.3 | 0.8 |
| Liver Cancer Research | 0.0 | 0.0 |
| Genetics RNA1025 | | |
| Liver Cancer Research | 2.8 | 3.2 |
| Genetics PNA1026 | | |
| Paired Liver Cancer Tissue Research | 0.2 | 0.4 |
| Genetics PNA 6004-T | | |
| Paired Liver Tissue Research | 2.1 | 0.3 |
| Genetics RNA 6004-N | | |
| Paired Liver Cancer Tissue Research | 1.1 | 3.9 |
| Genetics RNA 6005-T | | |
| Paired Liver Tissue Research | 0.5 | 0.0 |
| Genetics RNA 6005-N | | |
| Normal Bladder GENPAK 061001 | 13.1 | 10.7 |
| Bladder Cancer Research | 1.2 | 2.5 |
| Genetics PNA1023 | | |
| Bladder Cancer INVITROGEN | 3.5 | 4.7 |
| A302173 | | |
| 87071 Bladder Cancer (ODO4718-01) | 6.1 | 7.6 |
| 87072 Bladder Normal Adjacent | 33.9 | 24.7 |
| (ODO4718-03) | | |
| Normal Ovary Res. Gen. | 43.5 | 48.0 |
| Ovarian Cancer GENPAK 064008 | 32.1 | 26.4 |
| 87492 Ovary Cancer (ODO4768-07) | 4.6 | 3.3 |
| 87493 Ovary NAT (ODO4768-08) | 5.7 | 4.7 |
| Normal Stomach GENPAK 061017 | 13.6 | 16.7 |
| Gastric Cancer Clontech 9060358 | 12.1 | 16.5 |
| NAT Stomach Clontech 9060359 | 8.5 | 9.9 |
| Gastric Cancer Clontech 9060395 | 14.5 | 12.9 |
| NAT Stomach Clontech 9060394 | 14.1 | 14.1 |
| Gastric Cancer Clontech 9060397 | 6.3 | 8.2 |
| NAT Stomach Clontech 9060396 | 3.0 | 3.3 |
| Gastric Cancer GENPAK 064005 | 11.9 | 6.4 |

TABLE 13
Panel 3D

| | Relative | | Relative |
|---|---|---|---|
| | Expression (%) | | Expression (%) |
| | 3dx4tm5l35f | | 3dx4tm5135f |
| Tissue Name | _ag902_a1 | Tissue Name | _ag902_a1 |
| 94905 Daoy_Medulloblast | 4.8 | 94954_Ca Ski_Cervical | 0.0 |
| oma/Cerebellum sscDNA | | epidermoid carcinoma | |
| | | (metastasis)_sscDNA | |
| 94906_TE671_Medulloblas | 39.9 | 94955_ES-2_Ovarian | 0.0 |
| tom/Cerebellum sscDNA | | clear cell | |
| | | carcinoma_sscDNA | |
| 94907_D283 | 49.5 | 94957_Ramos/6h stim_“; | 0.0 |
| Med_Medulloblastoma/Cer | | Stimulated with | |
| ebellum_sscDNA | | PMA/ionomycin 6h_sscDNA | |
| 94908_PFSK-1_Primitive | 0.0 | 94958_Ramos/14h stim_“; | 0.0 |
| Neuroectodermal/Cerebel | | Stimulated with | |
| lum_sscDNA | | PMA/ionomycin | |
| | | 14h_sscDNA | |
| 94909 XF-498_CNS_sscDNA | 0.0 | 94962_MEG-01_Chronic | 2.2 |
| | | myelogenous leukemia | |
| | | (megokaryoblast)_sscDNA | |
| 94910_SNB- | 12.6 | 94963_Raji_Burkitt's | 0.0 |
| 78_CNS/glioma_sscDNA | | lymphoma_sscDNA | |
| 94911_SF- | 0.8 | 94964_Daudi_Burkitt's | 0.8 |
| 268_CNS/glioblastoma_ss | | lymphoma_sscDNA | 0.8 |
| cDNA | | | |
| 94912_T98G_Glioblastoma | 0.0 | 94965_U266_B-cell | 0.0 |
| _sscDNA | | plasmacytoma/myeloma_ss | |
| | | cDNA | |
| 96776_SK-N- | 0.5 | 94968_CA46_Burkitt's | 1.3 |
| SH_Neuroblastoma | | lymphoma_sscDNA | 1.3 |
| (metastasis)_sscDNA | | | |
| 94913_SF- | 1.2 | 94970_RL_non-Hodgkin's | 0.0 |
| 295_CNS/glioblastoma_ss | | B-cell lymphoma_sscDNA | |
| cDNA | | | |
| 94914_Cerebellum_sscDNA | 8.1 | 94972_JM1_pre-B-cell | 0.0 |
| | | lymphoma/leukemia_sscDNA | |
| 96777_Cerebellum_sscDNA | 4.4 | 94973_Jurkat_T_cell | 0.0 |
| | | leukemia_sscDNA | |
| 94916_NCI- | 0.7 | 94974_TF- | 0.6 |
| H292_Mucoepidermoid | | 1_Erythroleukemia_sscDNA | |
| lung carcinoma_sscDNA | | | |
| 94917_DMS-114_Small | 9.7 | 94975_HUT 78_T-cell | 0.8 |
| cell lung cancer_sscDNA | | lymphoma_sscDNA | |
| 94918_DMS-79_Small cell | 24.5 | 94977_U937_Histiocytic | 0.7 |
| lung | | lymphoma_sscDNA | |
| cancer/neuroendocrine_s | | | |
| scDNA | | | |
| 94919_NCI-H146_Small | 2.2 | 94980_KU- | 0.0 |
| cell lung | | 812_Myelogenous | |
| cancer/neuroendocrine_s | | leukemia_sscDNA | |
| scDNA | | | |
| 94920_NCI-H526_Small | 11.4 | 94981_769-P_Clear cell | 0.0 |
| cell lung | | renal carcinoma_sscDNA | |
| cancer/neuroendocrine_s | | | |
| scDNA | | | |
| 94921_NCI-N417_Small | 5.3 | 94983_Caki-2_Clear cell | 5.8 |
| cell lung | | renal carcinoma_sscDNA | |
| cancer/neuroendocrine_s | | | |
| scDNA | | | |
| 94923_NCI-H82_Small | 13.2 | 94984_SW 839_Clear cell | 1.7 |
| cell lung | | renal carcinoma sscDNA | |
| cancer/neuroendocrine_s | | | |
| scDNA | | | |
| 94924_NCI-H157_Squamous | 0.0 | 94986_G401_Wilms | 25.2 |
| cell lung cancer | | tumor_sscDNA | |
| (metastasis)_sscDNA | | | |
| 94925_NCI-H11S5 Large | 16.2 | 94987_Hs766T_Pancreatic | 3.9 |
| cell lung | | carcinoma (LN | |
| cancer/neuroendocrine_s | | metastasis)_sscDNA | |
| scDNA | | | |
| 94926_NCI-H1299_Large | 2.4 | 94988_CAPAN- | 3.1 |
| cell lung | | 1_Pancreatic | |
| cancer/neuroendocrine_s | | adenocarcinoma (liver | |
| scDNA | | metastasis)_sscDNA | |
| 94927_NCI-H727_Lung | 1.7 | 94989_SU86.86_Pancreatic | 26.0 |
| carcinoid_sscDNA | | carcinoma (liver | |
| | | metastasis)_sscDNA | |
| 94928_NCI-UMC-11_Lung | 3.1 | 94990_BxPC-3_Pancreatic | 0.0 |
| carcinoid_sscDNA | | adenocarcinoma_sscDNA | |
| 94929_LX-1_Small cell | 5.2 | 94991_HPAC_Pancreatic | 2.9 |
| lung cancer_sscDNA | | adenocarcinoma_sscDNA | |
| 94930_Colo-205_Colon | 1.6 | 94992_MIA PaCa- | 0.0 |
| cancer_sscDNA | | 2_Pancreatic | |
| | | carcinoma_sscDNA | |
| 94931_KM12_Colon | 0.0 | 94993_CFPAC- | 6.3 |
| cancer_sscDNA | | 1_Pancreatic ductal | |
| | | adenocarcinoma_sscDNA | |
| 94932_KM20L2_Colon | 3.4 | 94994_PANC-1_Pancreatic | 8.5 |
| cancer_sscDNA | | epithelioid ductal | |
| | | carcinoma_sscDNA | |
| 94933_NCI-H716_Colon | 100.0 | 94996_T24_Bladder | 0.0 |
| cancer_sscDNA | | carcinma (transitional | |
| | | cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 0.0 |
| adenocarcinoma_sscDNA | | carcinoma_sscDNA | |
| 94937_LS 174T_Colon | 8.8 | 94999_UM-UC-3_Bladder | 0.0 |
| adenocarcinoma_sscDNA | | carcinma (transitional | |
| | | cell)_sscDNA | |
| 94938_SW-948_Colon | 0.0 | 95000_A204_Rhabdomyosar | 0.5 |
| adenocarcinoma_sscDNA | | coma_sscDNA | |
| 94939_SW-480_Colon | 0.0 | 95001_HT- | 3.7 |
| adenocarcinoma_sscDNA | | 1080_Fibrosarcoma_sscDNA | |
| 94940_NCI-SNU-5_Gastric | 5.4 | 95002_MG- | 7.9 |
| carcinoma_sscDNA | | 63_Osteosarcoma | |
| | | (bone)_sscDNA | |
| 94941_KATO III_Gastric | 1.9 | 95003_SK-LMS- | 2.2 |
| carcinoma_sscDNA | | 1_Leiomyosarcoma | |
| | | (vulva)_sscDNA | |
| 94943_NCI-SNU- | 10.7 | 95004_SJRH30_Rhabdomyos | 33.4 |
| 16_Gastric | | arcoma (met to bone | |
| carcinoma_sscDNA | | marrow)_sscDNA | |
| 94944_NCT-SNU-1_Gastric | 0.0 | 95005_A431_Epidermoid | 0.0 |
| carcinoma_sscDNA | | carcinoma_sscDNA | |
| 94946_RF-1_Gastric | 0.7 | 95007_WM266- | 0.8 |
| adenocarcinoma_sscDNA | | 4_Melanoma_sscDNA | |
| 94947_RF-48_Gastric | 1.4 | 95010_DU 145_Prostate | 0.8 |
| adenocarcinoma_sscDNA | | carcinoma (brain | |
| | | metastasis)_sscDNA | |
| 96778_MKN-45_Gastric | 0.9 | 95012_MDA-MB-468_Breast | 0.0 |
| carcinoma_sscDNA | | adenocarcinoma_sscDNA | |
| 94949_NCI-N87_Gastric | 0.0 | 95013_SCC-4 Squamous | 1.3 |
| carcinoma_sscDNA | | cell carcinoma of | |
| | | tongue_sscDNA | |
| 94951_OVCAR-5_Ovarian | 1.0 | 95014_SCC-9_Squamous | 0.0 |
| carcinoma_sscDNA | | cell carcinoma of | |
| | | tongue_sscDNA | |
| 94952_RL95-2_Uterine | 0.0 | 95015_SCC-15_Squamous | 0.0 |
| carcinoma_sscDNA | | cell carcinoma of | |
| | | tongue_sscDNA | |
| 94953_HelaS3_Cervical | 0.7 | 95017_CAL 27_Squamous | 0.0 |
| adenocarcinoma_sscDNA | | cell carcinoma of | |
| | | tongue_sscDNA | |

TABLE 14
Panel 4.1D

| | Relative | | Relative |
|---|---|---|---|
| | Expression (%) | | Expression (%) |
| | 4.1dx4tm662 | | 4.1dx4tm662 |
| Tissue Name | 2f_ag902_a1 | Tissue Name | 2f_ag902_a1 |
| 93768_Secondary | 0.0 | 93100_HUVEC | 0.0 |
| Th1_anti-CD28/anti-CD3 | | (Endothelial)_IL-1b | |
| 93769_Secondary | 0.0 | 93779_HUVEC | 1.5 |
| Th2_anti-CD28/anti-CD3 | | (Endothelial)_IFN gamma | |
| 93770_Secondary | 0.2 | 93102_HUVEC | 0.0 |
| Tri_anti-CD28/anti-CD3 | | (Endothelial)_TNF alpha + | |
| | | IFN gamma | |
| 93573_Secondary | 0.2 | 93101_HUVEC | 0.0 |
| Th1_resting day 4-6 in | | (Endothelial)_TNF alpha + | |
| IL-2 | | IL4 | |
| 93572_Secondary | 0.3 | 93781_HUVEC | 0.0 |
| Th2_resting day 4-6 in | | (Endothelial)_IL-11 | |
| IL-2 | | | |
| 93571_Secondary | 0.0 | 93583_Lung | 0.4 |
| Tr1_resting day 4-6 in | | Microvascular | |
| IL-2 | | Endothelial Cells_none | |
| 93568_primary Th1_anti- | 0.0 | 93584_Lung | 0.3 |
| CD28/anti-CD3 | | Microvascular | |
| | | Endothelial Cells_TNFa | |
| | | (4 ng/ml) and IL1b (1 | |
| | | ng/ml) | |
| 93569_primary Th2_anti- | 0.1 | 92662_Microvascular | 0.0 |
| CD28/anti-CD3 | | Dermal endothelium_none | |
| 93570_primary Tr1_anti- | 0.0 | 92663_Microsvasular | 0.0 |
| CD28/anti-CD3 | | Dermal endothelium_TNFa | |
| | | (4 ng/ml) and IL1b (1 | |
| | | ng/ml) | |
| 93565_primary | 0.4 | 93773_Bronchial | 2.3 |
| Th1_resting dy 4-6 in | | epithelium_TNFa (4 | |
| IL-2 | | ng/ml) and IL1b (1 | |
| | | ng/ml) ** | |
| 93566_primary | 0.0 | 93347_Small Airway | 1.6 |
| Th2_resting dy 4-6 in | | Epithelium none | |
| IL-2 | | | |
| 93567_primary | 0.0 | 93348_Small Airway | 0.4 |
| Tr1_resting dy 4-6 in | | Epithelium_TNFa (4 | |
| IL-2 | | ng/ml) and IL1b (1 | |
| | | ng/ml) | |
| 93351_CD45RA CD4 | 1.7 | 92668_Coronery Artery | 0.9 |
| lymphocyte_anti- | | SMC_resting | |
| CD28/anti-CD3 | | | |
| 93352_CD45RO CD4 | 0.2 | 92669_Coronery Artery | 1.3 |
| lymphocyte_anti- | | SMC_TNFa (4 ng/ml) and | |
| CD28/anti-CD3 | | IL1b (1 ng/ml) | |
| 93251_CD8 | 0.0 | 93107_astrocytes_restin | 13.3 |
| Lymphocytes_anti- | | g | |
| CD28/anti-CD3 | | | |
| 93353_chronic CD8 | 0.3 | 93108_astrocytes_TNFa | 9.8 |
| Lymphocytes 2ry_resting | | (4 ng/ml) and IL1b (1 | |
| dy 4-6 in IL-2 | | ng/ml) | |
| 93574_chronic CD8 | 0.0 | 92666_KU-812 | 0.0 |
| Lymphocytes | | (Basophil)_resting | |
| 2ry_activated CD3/CD28 | | | |
| 93354_CD4_none | 0.0 | 92667_KU-812 | 0.0 |
| | | (Basophil)_PMA/ionoycin | |
| 93252_Secondary | 0.3 | 93579_CCD1106 | 0.3 |
| Th1/Th2/Tr1_anti-CD95 | | (Keratinocytes)_none | |
| CH11 | | | |
| 93103_LAK cells_resting | 0.0 | 93580_CCD1106 | 0.0 |
| | | (Keratinocytes)_TNFa | |
| | | and IFNg ** | |
| 93788_LAK cells_IL-2 | 0.0 | 93791_Liver Cirrhosis | 0.1 |
| 93787_LAK cells_IL- | 0.0 | 93577_NCI-H292 | 0.5 |
| 2 + IL-12 | | | |
| 93789_LAK cells_IL- | 0.5 | 93358_NCI-H292_IL-4 | 0.0 |
| 2 + IFN gamma | | | |
| 93790_LAK cells_IL-2 + | 0.7 | 93360_NCI-H292_IL-9 | 1.0 |
| IL-18 | | | |
| 93104_LAK | 0.0 | 93359_NCI-H292_IL-13 | 0.0 |
| cells_PMA/ionomycin and | | | |
| IL-18 | | | |
| 93578_NK Cells IL- | 0.8 | 93357_NCI-H292_IFN | 0.3 |
| 2_resting | | gamma | |
| 93109_Mixed Lymphocyte | 0.8 | 93777_HPAEC_- | 0.0 |
| Reaction_Two Way MLR | | | |
| 93110_Mixed Lymphocyte | 0.0 | 93778_HPAEC_IL-1 | 0.0 |
| Reaction_Two Way MLR | | beta/TNA alpha | |
| 93111_Mixed Lymphocyte | 0.0 | 93254_Normal Human Lung | 4.2 |
| Reaction_Two Way MLR | | Fibroblast_none | |
| 93112_Mononuclear Cells | 0.0 | 93253_Normal Human Lung | 14.4 |
| (PBMCs)_resting | | Fibroblast_TNFa (4 | |
| | | ng/ml) and IL-1b (1 | |
| | | ng/ml) | |
| 93113_Mononuclear Cells | 0.0 | 93257_Normal Human Lung | 4.1 |
| (PBMCs)_PWM | | Fibroblast_IL-4 | |
| 93114_Mononuclear Cells | 0.0 | 93256_Normal Human Lung | 3.0 |
| (PBMCS)_PHA-L | | Fibroblast_IL-9 | |
| 93249_Ramos (B | 0.2 | 93255_Normal Human Lung | 4.2 |
| cell)_none | | Fibroblast_IL-13 | |
| 93250_Ramos (B | 0.0 | 93258_Normal Human Lung | 4.2 |
| cell)_ionomycin | | Fibroblast_IFN gamma | |
| 93349_B lymphocytes_PWM | 0.0 | 93106_Dermal | 3.6 |
| | | Fibroblasts | |
| | | CCD1070_resting | |
| 93350_B | 0.2 | 93361_Dermal | 1.5 |
| lymphoytes_CD40L and | | Fibroblasts CCD1070_TNF | |
| IL-4 | | alpha 4 ng/ml | |
| 92665_EOL-1 | 0.0 | 93105_Dermal | 2.2 |
| (Eosinophil)_dbcAMP | | Fibroblasts CCD1070_IL- | |
| differentiated | | 1 beta 1 ng/ml | |
| 93248_EOL-1 | 0.0 | 93772_dermal | 9.5 |
| (Eosinophil)_dbcAMP/PMA | | fibroblast_IFN gamma | |
| ionomycin | | | |
| 93356_Dendritic | 0.0 | 93771_dermal | 30.2 |
| Cells_none | | fibroblast_IL-4 | |
| 93355_Dendritic | 100.0 | 93892_Dermal | 6.1 |
| Cells_LPS 100 ng/ml | | fibroblasts_none | |
| 93775_Dendritic | 0.0 | 99202_Neutrophils_TNFa + | 0.0 |
| Cells_anti-CD40 | | LPS | |
| 93774_Monocytes_resting | 0.0 | 99203_Neutrophils_none | 0.0 |
| 93776_Monocytes_LPS 50 | 0.8 | 735010_Colon_normal | 0.9 |
| ng/ml | | | |
| 93581_Macrophages_resting | 6.9 | 735019_Lung_none | 3.1 |
| 93582_Macrophages_LPS | 1.0 | 64028-1_Thymus_none | 8.3 |
| 100 ng/ml | | | |
| 93098_HUVEC | 0.0 | 64030-1_Kidney_none | 1.5 |
| (Endothelial)_none | | | |
| 93099_HUVEC | 0.0 | | |
| (Endothelial)_starved | | | |

Panel 1.2 Summary: Ag902 Results from two replicate experiments using the same probe/primer set are in reasonable agreement. Expression in adipose is skewed by the presence of genomic DNA contamination in this sample. The AC068507A gene encodes a putative cell-surface protein of the immunoglobulin superfamily. This gene is expressed at varying levels across the majority of samples on this panel. However, expression of the AC068507A gene is highest in skeletal muscle (CT value=24) and adrenal gland (CT value=25-26). As a putative cell-surface protein with a cytoplasmic domain, the AC068507A gene product may therefore bind extracellular ligands and play a role in signal transduction. Thus, this gene may be a drug target for the treatment of diseases involving skeletal muscle or the adrenal gland. In addition, AC068507A gene expression is also high in the following metabolically related tissues: pancreas, pituitary gland, thyroid, and heart. This observation may suggest that the AC068507A gene plays a role in normal metabolic and neuroendocrine function and that disregulation of this gene may contribute to metabolic diseases (such as obesity and diabetes) or neuroendocrine disorders.

Expression of the AC068507A gene is also high in many regions of the brain, including amygdala, thalamus, cerebellum, hippocampus and cerebral cortex. The protein encoded by the AC068507A gene is a homolog of NOPE, which appears to function as a guidance receptor in the developing CNS (refs 1 and 2). Similarly, the AC068507A gene is also expressed in the developing brain, as well as in the mature CNS. Therefore, manipulation of levels of the AC068507A protein may be of use in inducing and/or directing a compensatory synaptogenic response to neuronal death in the treatment of Alzheimer's disease, Parkinson's disease, Huntington's disease, spinocerebellar ataxia, progressive supranuclear palsy, ALS, head trauma, stroke, or any other disease/condition associated with neuronal loss.

Interestingly, expression of the AC068507A gene appears to be higher in fetal tissues compared to adult tissues, especially in fetal liver, lung, brain and kidney. This pattern of expression suggests that the AC068507A gene might be involved in tissue development and hence therapeutic modulation of the expression of this gene could be of use in the regeneration of disease tissue suffering degeneration.

Panel 2D Summary: Ag902 Results from two replicate experiments using the same probe/primer set are in good agreement. The AC068507A gene is expressed in a number of tissues in panel 2D. Of particular interest is the over-expression of the AC068507A gene in 7/9 kidney cancer samples, and to a lesser degree in colon cancer, when compared to their normal adjacent tissues. Thus, the expression of the AC068507A gene is of potential utility in the diagnosis of kidney cancer. In addition, therapeutic modulation of this gene using inhibitory monoclonal antibodies or small molecule therapeutics might be of use in the treatment of kidney cancer.

Panel 3D Summary: AR902 The AC068507A gene is expressed in a number of cancer cell line samples in panel 3D. Highest expression of this gene was detected in a colon cancer cell line (CT value=30.1), which may confirm its potential role in colon cancer. The AC068507A gene is also expressed in kidney cancer cell lines in Panel 3D, consistent with the results obtained in Panel 2D. These observations suggest that this gene may be playing a role in the pathogenesis of kidney and colon cancer, or other cancers. Therefore, therapeutic modulation of the AC068507A gene using inhibitory monoclonal antibodies or small molecule therapeutics might be of use in the treatment of multiple types of cancer.

Panel 4.1D Summary: Ag902 The AC068507A transcript is induced by LPS (100X) in dendritic cells and by IL-4 in dermal fibroblasts. There is very little expression in normal tissues represented in panel 4. This transcript codes for a putative plasma membrane molecule has high homology to guidance receptors (see reference 1). Dendritic cells and dermal fibroblasts may utilize the protein encoded for by this transcript as a receptor that controls interactions between themselves and other cell types perhaps in the context of antigen presentation, apoptosis (see reference 2) or unique functions. Antibody or small molecule therapeutics designed against the protein encoded for by this transcript could inhibit or block inflammation in diseases such as asthma, arthritis, psoriasis, allergy and other diseases in which dendritic cell or dermal fibroblasts play important roles.

The novel mouse gene Nope was identified due to its proximity to the Punc gene on chromosome 9. With a domain structure of four immunoglobulin domains, five fibronectin type III repeats, a single transmembrane domain, and a cytoplasmic domain, Nope encodes a new member of the immunoglobulin superfamily of cell surface proteins. It displays a high level of similarity to Punc, as well as to guidance receptors such as the Deleted in Colorectal Cancer protein and Neogenin. Nope is expressed during embryonic development in the notochord, in developing skeletal muscles, and later in the ventricular zone of the nervous system. In the adult brain, Nope can be detected in the hippocampus. Radiation hybrid mapping of Nope, Punc, and Neogenin placed all three genes in close vicinity on mouse chromosome 9. See generally, Salbaum J. M., Kappen C. (2000) Cloning and expression of nope, a new mouse gene of the immunoglobulin superfamily related to guidance receptors. Genomics 64: 15-23.

The formation of precise connections between neurons and their targets during development is dependent on extracellular guidance cues that allow growing axons to navigate to their targets. One family of such guidance molecules, conserved across all species examined, is that of the netrin/UNC-6 proteins. Netrins act to both attract and repel the growing axons of a broad range of neuronalcell types during development and are also involved in controlling neuronal cell migration. These actions are mediated by specific receptor complexes containing either the colorectal cancer (DCC) or neogenin protein, in the case of the attractive receptor, or UNC-5-related proteins, in the case of the repellent receptor. Recent work has identified a key role for intracellular cyclic nucleotide levels in regulating the nature of the response of the growing axon to netrins as either attractive or repulsive. Netrin-DCC signaling has also been shown to regulate cell death in epithelial cells in vitro, raising the interesting possibility that netrins may also regulate cell death in the developing nervous system. See generally, Livesey F. J. (1999) Netrins and netrin receptors. Cell Mol. Life Sci. 56: 62-68.

### EXAMPLE 3

### Quantitative expression analysis of NOV2 expression in various cells and tissues

Expression of gene SC101760703_A was assessed using the primer-probe set Ag1311, described in Table BA. Results of the RTQ-PCR runs are shown in Tables BB and BC.

TABLE 15
Probe Name Ag1311

| | | | | Start |
|---|---|---|---|---|
| Primers | Sequences | TM | Length | Position |
| Forward | 5′-TCCAGTACCTGAGCTGGTAGTT-3′ | 58 | 22 | 594 |
| | SEQ ID NO:51 | | | |
| Probe | TET -5′-TGGACCGAGAGAACCGCTCACACTAT- | 70 | 26 | 626 |
| | 3′-TAMRA | | | |
| | SEQ ID NO:52 | | | |
| Reverse | 5′-ATCATAGGCCTCCAGCTGTAG-3′ | 58.5 | 21 | 655 |
| | SEQ ID NO:53 | | | |

TABLE 16
Panel 1.2

| | Relative | | Relative |
|---|---|---|---|
| | Expression (%) | | Expression (%) |
| | 1.2tm1451t_ | | 1.2tm1451t_ |
| Tissue Name | ag1311 | Tissue Name | ag1311 |
| Endothelial cells | 30.1 | Renal ca. 786-0 | 0.0 |
| Fetal heart | 100.0 | Renal ca. A498 | 0.0 |
| Pancreas | 3.3 | Renal ca. RXF 393 | 0.0 |
| Pancreatic ca. CAPAN 2 | 0.0 | Renal ca. ACEN | 0.0 |
| Adrenal Gland (new | 8.4 | Renal ca. UO-31 | 0.1 |
| lot*) | | | |
| Thyroid | 2.7 | Renal ca. TK-10 | 0.0 |
| Salavary gland | 4.8 | Liver | 5.8 |
| Pituitary gland | 4.8 | Liver (fetal) | 3.3 |
| Brain (fetal) | 10.9 | Liver ca. (hepatoblast) | 0.2 |
| | | HepG2 | |
| Brain (whole) | 4.7 | Lung | 4.9 |
| Brain (amygdala) | 3.8 | Lung (fetal) | 7.0 |
| Brain (cerebellum) | 4.5 | Lung ca. (small cell) | 0.2 |
| | | LX-1 | |
| Brain (hippocampus) | 7.2 | Lung ca. (small cell) | 0.9 |
| | | NCI-H69 | |
| Brain (thalamus) | 2.9 | Lung ca. (s.cell var.) | 0.0 |
| | | SHP-77 | |
| Cerebral Cortex | 25.7 | Lung ca. (large | 1.1 |
| | | cell) NCI-H460 | |
| Spinal cord | 4.2 | Lung ca. (non-sm. cell) | 0.1 |
| | | A549 | |
| CNS ca. (glio/astro) | 0.3 | Lung ca. (non-s.cell) | 0.2 |
| U87-MG | | NCI-H23 | |
| CNS ca. (glio/astro) U- | 2.2 | Lung ca (non-s.cell) | 4.4 |
| 118-MG | | HOP-62 | |
| CNS ca. (astro) SW1783 | 1.0 | Lung ca. (non-s.cl) | 1.3 |
| | | NCI-H522 | |
| CNS ca.* (neuro; met) | 22.5 | Lung ca. (squam.) SW | 0.2 |
| SK-N-AS | | 900 | |
| CNS ca. (astro) SF-539 | 2.1 | Lung ca. (squam.) NCI- | 0.6 |
| | | H596 | |
| CNS ca. (astro) SNB-75 | 0.7 | Mammary gland | 12.6 |
| CNS ca. (glio) SNB-19 | 4.6 | Breast ca.* (pl. | 0.0 |
| | | effusion) MCF-7 | |
| CNS ca. (glio) U251 | 0.2 | Breast ca.* (pl.ef) | 0.0 |
| | | MDA-MB-231 | |
| CNS ca. (glio) SF-295 | 0.2 | Breast ca.* (pl. | 0.0 |
| | | effusion) T47D | |
| Heart | 36.9 | Breast ca. BT-549 | 0.1 |
| Skeletal Muscle (new | 5.8 | Breast ca. MDA-N | 0.2 |
| lot*) | | | |
| Bone marrow | 0.3 | Ovary | 41.5 |
| Thymus | 2.2 | Ovarian ca. OVCAR-3 | 0.3 |
| Spleen | 2.7 | Ovarian ca. OVCAR-4 | 0.0 |
| Lymph node | 5.0 | Ovarian ca. OVCAR-5 | 0.1 |
| Colorectal | 3.1 | Ovarian ca. OVCAR-8 | 1.0 |
| Stomach | 9.4 | Ovarian ca. IGROV-1 | 0.0 |
| Small intestine | 9.3 | Ovarian ca.* (ascites) | 4.0 |
| | | SK-OV-3 | |
| Colon ca. SW480 | 0.0 | Uterus | 12.4 |
| Colon ca.* (SW480 | 0.1 | Placenta | 19.6 |
| met) SW620 | | | |
| Colon ca. HT29 | 0.0 | Prostate | 7.0 |
| Colon ca. HCT-116 | 0.0 | Prostate ca.* (bone | 0.1 |
| | | met) PC-3 | |
| Colon ca. CaCo-2 | 0.4 | Testis | 3.2 |
| 83219 CC Well to Mod | 4.1 | Melanoma Hs688 (A) .T | 4.6 |
| Diff (ODO3866) | | | |
| Colon ca. HCC-2998 | 0.1 | Melanoma* (met) | 3.0 |
| | | Hs688 (B) .T | |
| Gastric ca.* (liver | 0.0 | Melanoma UACC-62 | 0.3 |
| met) NCI-N87 | | | |
| Bladder | 9.3 | Melanoma M14 | 0.1 |
| Trachea | 2.5 | Melanoma LOX IMVI | 0.0 |
| Kidney | 7.6 | Melanoma* (met) SK-MEL- | |
| | | 5 | |
| Kidney (fetal) | 26.8 | Adipose | 11.5 |

TABLE 17
Panel 4D

| | Relative | | Relative |
|---|---|---|---|
| | Expression (%) | | Expression (%) |
| | 4Dtm1887t_ | | 4Dtm1887t_ |
| Tissue Name | ag1311 | Tissue Name | ag1311 |
| 93768_Secondary | 0.4 | 93100_HUVEC | 22.4 |
| Th1_anti-CD28/anti-CD3 | | (Endothelial)_IL-1b | |
| 93769_Secondary | 2.0 | 93779_HUVEC | 100.0 |
| Th2_anti-CD28/anti-CD3 | | (Endothelial)_IFN gamma | |
| 93770_Secondary | 1.6 | 93102_HUVEC | |
| Tr1_anti-CD28/anti-CD3 | | (Endothelial)_TNF alpha + | 11.7 |
| | | IFN gamma | |
| 93573_Secondary | 0.2 | 93101_HUVEC | 24.5 |
| Th1_resting day 4-6 in | | (Endothelial)_TNF alpha + | |
| IL-2 | | IL4 | |
| 93572_Secondary | 0.1 | 93781_HUVEC | 38.2 |
| Th2_resting day 4-6 in | | (Endothelial)_IL-11 | |
| IL-2 | | | |
| 93571_Secondary | 0.3 | 93583_Lung | 54.3 |
| Tr1_resting day 4-6 in | | Microvascular | |
| IL-2 | | Endothelial Cells_none | |
| 93568_primary Th1_anti- | 0.7 | 93584_Lung | 24.3 |
| CD28/anti-CD3 | | Microvascular | |
| | | Endothelial Cells_TNFa | |
| | | (4 ng/ml) and IL1b (1 | |
| | | ng/ml) | |
| 93569_primary Th2_anti- | 1.2 | 92662_Microvascular | 79.0 |
| CD28/anti-CD3 | | Dermal endothelium_none | |
| 93570_primary Tr1_anti- | 0.6 | 92663_Microsvasular | 51.4 |
| CD28/anti-CD3 | | Dermal endothelium_TNFa | |
| | | (4 ng/ml) and IL1b (1 | |
| | | ng/ml) | |
| 93565_primary | 2.8 | 93773_Bronchial | 0.0 |
| Th1_resting dy 4-6 in | | epithelium_TNFa (4 | |
| IL-2 | | ng/ml) and IL1b (1 | |
| | | ng/ml) ** | |
| 93566_primary | 2.4 | 93347_Small Airway | 0.0 |
| Th2_resting dy 4-6 in | | Epithelium_none | |
| IL-2 | | | |
| 93567_primary | 0.7 | 93348_Small Airway | 1.2 |
| Tr1_resting dy 4-6 in | | Epithelium_TNFa (4 | |
| IL-2 | | ng/ml) and IL1b (1 | |
| | | ng/ml) | |
| 93351_CD45RA CD4 | 11.8 | 92668_Coronery Artery | 2.1 |
| lymphocyte_anti- | | SMC_resting | |
| CD28/anti-CD3 | | | |
| 93352_CD45RO CD4 | 2.2 | 92669_Coronery Artery | 3.5 |
| lymphocyte_anti- | | SMC_TNFa (4 ng/ml) and | |
| CD28/anti-CD3 | | IL1b (1 ng/ml) | |
| 93251_CD8 | 1.4 | 93107_astrocytes_resting | 19.3 |
| Lymphocytes_anti- | | | |
| CD28/anti-CD3 | | | |
| 93353_chronic CD8 | 1.2 | 93108_astrocytes_TNFa | 8.2 |
| Lymphocytes 2ry_resting | | (4 ng/ml) and IL1b (1 | |
| dy 4-6 in IL-2 | | ng/ml) | |
| 93574_chronic CD8 | 0.3 | 92666_KU-812 | 0.3 |
| Lymphocytes | | (Basophil)_resting | |
| 2ry activated CD3/CD28 | | | |
| 93354_CD4_none | 8.5 | 92667_KU-812 | 0.0 |
| | | (Basophil)_PMA/ionoycin | |
| 93252_Secondary | 0.5 | 93579_CCD1106 | 0.7 |
| Th1/Th2 /Tr1_anti-CD95 | | (Keratinocytes)_none | |
| CH11 | | | |
| 93103_LAK cells_resting | 5.9 | 93580_CCD1106 | 0.6 |
| | | (Keratinocytes)_TNFa | |
| | | and IFNg** | |
| 93788_LAK cells_IL-2 | 0.6 | 93791_Liver Cirrhosis | 3.3 |
| 93787_LAK cells_IL- | 2.2 | 93792_Lupus Kidney | 1.4 |
| 2 + IL-12 | | | |
| 93789_LAK cells_IL- | 2.5 | 93577_NCI-H292 | 1.6 |
| 2 + IFN gamma | | | |
| 93790_LAK cells_IL-2 + | 1.3 | 93358_NCI-H292_IL-4 | 1.4 |
| IL-18 | | | |
| 93104_LAK | 8.5 | 93360_NCI-H292_IL-9 | 0.7 |
| cells_PMA/ionomycin and | | | |
| IL-18 | | | |
| 93578_NK Cells IL- | 3.7 | 93359_NCI-H292_IL-13 | 1.7 |
| 2_resting | | | |
| 93109_Mixed Lymphocyte | 1.7 | 93357_NCI-H292_IFN | 2.1 |
| Reaction_Two Way MLR | | gamma | |
| 93110_Mixed Lymphocyte | 2.4 | 93777_HPAEC_- | 56.6 |
| Reaction_Two Way MLR | | | |
| 93111_Mixed Lymphocyte | 2.4 | 93778_HPAEC_IL-1 | 41.8 |
| Reaction_Two Way MLR | | beta/TNA alpha | |
| 93112_Mononuclear Cells | 1.4 | 93254_Normal Human Lung | 22.4 |
| (PBMCs)_resting | | Fibroblast_none | |
| 93113_Mononuclear Cells | 1.2 | 93253_Normal Human Lung | 14.8 |
| (PBMCs)_PWM | | Fibroblast_TNFa (4 | |
| | | ng/ml) and IL-1b (1 | |
| | | ng/ml) | |
| 93114_Mononuclear Cells | 1.7 | 93257_Normal Human Lung | 33.4 |
| (PBMCs)_PHA-L | | Fibroblast_IL-4 | |
| 93249_Ramos (B | 0.3 | 93256_Normal Human Lung | 23.2 |
| cell)_none | | Fibroblast_IL-9 | |
| 93250_Ramos (B | 1.4 | 93255_Normal Human Lung | 50.7 |
| cell)_ionomycin | | Fibroblast_IL-13 | |
| 93349_B lymphocytes_PWM | 1.6 | 93258_Normal Human Lung | 52.1 |
| | | Fibroblast_IFN gamma | |
| 93350_B | 1.0 | 93106_Dermal | 23.5 |
| lymphoytes_CD40L and | | Fibroblasts | |
| IL-4 | | CCD1070_resting | |
| 92665_EOL-1 | 0.1 | 93361 Dermal | 19.3 |
| (Eosinophil)_dbcAMP | | Fibroblasts CCD1070_TNF | |
| differentiated | | alpha 4 mg/ml | |
| 93248_EOL-1 | 0.0 | 93105_Dermal | 19.9 |
| (Eosinophil)_dbcAMP/PMA | | Fibroblasts CCD1070_IL- | |
| ionomycin | | 1 beta 1 ng/ml | |
| 93356_Dendritic | 2.8 | 93772_dermal | 29.7 |
| Cells_none | | fibroblast_IFN gamma | |
| 93355_Dendritic | 0.6 | 93771_dermal | 62.0 |
| Cells_LPS 100 ng/ml | | fibroblast_IL-4 | |
| 93775_Dendritic | 0.9 | 93259_IBD Colitis 1** | 9.7 |
| Cells_anti-CD40 | | | |
| 93774_Monocytes_resting | 1.0 | 93260_IBD Colitis 2 | 2.1 |
| 93776_Monocytes_LPS 50 | 1.1 | 93261_IBD Crohns | 2.6 |
| ng/ml | | | |
| 93581_Macrophages_resting | 1.7 | 735010_Colon_normal | 20.0 |
| 93582_Macrophages_LPS | 1.4 | 735019_Lung_none | 75.3 |
| 100 ng/ml | | | |
| 93098_HUVEC | 45.7 | 64028-1_Thymus_none | 29.7 |
| (Endothelial)_none | | | |
| 93099_HUVEC | 75.8 | 64030-1_Kidney_none | 32.5 |
| (Endothelial)_starved | | | |

Panel 1.2 Summary: Ag1311 The protein encoded by the SC101760703_A gene is homologous to cadherin, a cell-adhesion protein. The SC101760703_A gene is highly expressed in a number of samples on panel 1.2. Specifically, the highest expression is detected in fetal heart (CT value=22.6), although it is also highly expressed in adult heart. This may suggest a potential role for the SC101760703_A gene in cardiovascular diseases such as cardiomyopathy, atherosclerosis, hypertension, congenital heart defects, aortic stenosis, atrial septal defect (asd), atrioventricular (a-v) canal defect, ductus arteriosus, pulmonary stenosis, subaortic stenosis, ventricular septal defect (vsd), and valve diseases. Overall, SC101760703_A gene expression is associated with normal tissues rather than cancer cell lines. Loss of function of the related E-cadherin protein has been described in many tumors, along with an increased invasiveness and a decreased prognosis of many carcinomas, including tumors of endocrine glands and their target systems (ref 1). Thus, the SC101760703_A gene product might similarly be useful as a protein therapeutic to treat a variety of tumors, since it is found in normal cells but missing from cancer cells.

In addition, the SC101760703_A gene is highly expressed in pituitary gland, adrenal gland, thyroid, pancreas, skeletal muscle, and liver, reflecting the widespread role of cadherins in cell-cell adhesion. This observation may suggest that the SC101760703_A gene plays a role in normal metabolic and neuroendocrine function and that disregulated expression of this gene may contribute to metabolic diseases (such as obesity and diabetes) or neuroendocrine disorders. Please note that expression in adipose is skewed by the presence of genomic DNA contamination in this sample.

Expression of the SC101760703_A gene is also high in many regions of the brain, including the amygdala, thalamus, cerebellum, and cerebral cortex, with highest expression in the hippocampus. Expression is also detected in the spinal cord. Cadherins can act as axon guidance and cell adhesion proteins, specifically during development and in the response to injury (ref 2). Manipulation of levels of this protein may be of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Panel 4D Summary: Ag1311 Expression of the SC101760703_A transcript is primarily in endothelial cells and in fibroblasts. However, this transcript is also expressed in the kidney, thymus, lung and colon. The expression of the transcript is high in normal tissue and untreated cells and is not affected by most treatments with the exception of IL-1 alpha and TNFbeta, which reduce expression of the transcript by half in treated HUVECs and reduce expression 10-fold in gamma interferon treated HUVECs. Therefore, the protein encoded for by the SC101760703_A gene may be important in normal function of endothelium and fibroblasts. Protein therapeutics designed with the protein encoded for by this transcript could reduce or block inflammation in diseases such as asthma, emphysema, allergy, arthritis, IBD and psoriasis.

Cell-cell adhesion, as mediated by the cadherin-catenin system, is a prerequisite for normal cell function and the preservation of tissue integrity. With recent progress in our understanding, beta-catenin as a component of a complex signal transduction pathway may serve as a common switch in central processes that regulate cellular differentiation and growth. The function of the cadherin-catenin system in cell adhesion as well as in intracellular signaling, appears to be subjected to multifactorial control by a variety of different mechanisms, and data on a hormonal control of these signaling pathways, even though scarce to date, suggest an important regulatory influence in many cellular systems. Loss of E-cadherin-catenin function was described in many tumors along with an increased invasiveness and a decreased prognosis of many carcinomas, including tumors of endocrine glands and their target systems, and a causal role of this loss-of-function in the multifactorial process of tumorigenesis was recently proven in genetic mouse models. Modification of E-caderin-catenin function in endocrine and nonendocrine tumors may involve germline and somatic gene mutations, epigenetic mechanisms such as gene silencing due to promotor-hypermethylation, and posttranscriptional events, likely to be involved in many endocrine tissues and their target organs. Such events may converge on nuclear activation of oncogenes such as c-myc by the beta-catenin/TCF4 complex. The expression and functional status of the components of the cadherin-catenin system may serve as prognostic markers for endocrine and nonendocrine tumors. The frequent involvement of functional dysregulation in many tumors raises hopes that better definition of the regulation of all components of the cadherin-catenin system and their response to extracellular modulators may eventually lead to new therapeutic approaches for these tumors and help to prevent, more specifically, growth, invasion, and metastasis of these carcinomas. See generally, Potter E., Bergwitz C., Brabant G. (1999) The cadherin-catenin system: implications for growth and differentiation of endocrine tissues. Endocr. Rev. 20: 207-239.

The formation of the myriad of neuronal connections within the vertebrate nervous system relies on expression of molecular tags that match extending axon populations with synaptic target sites. Recent work suggests that cadherins, a group of calcium-dependent cell adhesion molecules, are candidates to serve such a role. The diversity of the cadherin family in the nervous system allows for a multitude of interactions to specify neuronal connections. Specific cadherin types demarcate subpopulations of developing axons that interconnect within neuronal circuits. Expression of different cadherin species at select synapse populations raises exciting prospects for this molecule class in controlling adhesive interactions during synaptogenesis and plasticity. Regulation of cadherin-mediated adhesive strength is an attractive mechanism to explain the different cadherin functions in axon growth and at synapses. Ranscht B. (2000) Cadherins: molecular codes for axon guidance and synapse formation. Int. J. Dev. Neurosci. 18: 643-651.

### EXAMPLE 4

### Quantitative expression analysis of NOV4a expression in various cells and tissues

Expression of gene SC30236456_EXT1 was assessed using the primer-probe sets Ag1322 (identical sequence to Ag1322b) and Ag2071 (identical sequence to Ag2098), described in Tables CA and CB. Results of the RTQ-PCR runs are shown in Tables CC and CD.

TABLE 18
Probe Name Ag1322/Ag1322b

| | | | | Start |
|---|---|---|---|---|
| Primers | Sequences | TM | Length | Position |
| Forward | 5′-GGTATGTGCCTGCCCTATTATT-3′ SEQ ID NO:55 | 59.3 | 22 | 975 |
| Probe | FAM-5′-CCACCAGTATCATTAAGGATCTTTTACCTG-3′-TAMRA SEQ ID NO:56 | 64.8 | 30 | 997 |
| Reverse | 5′-GCCATTCTGTTTGCAATTATGT-3′ SEQ ID NO:57 | 59 | 22 | 1033 |

TABLE 19
Probe Name Ag2071/Ag2098

| | | | | Start |
|---|---|---|---|---|
| Primers | Sequences | TM | Length | Position |
| Forward | 5′-TGCCAGAAATTCATTTCCTAAA-3′ SEQ ID NO:58 | 58.7 | 22 | 2236 |
| Probe | FAM-5′-CCTTGGAAAGCCTGCCCACTAGTTTT-3′-TAMRA SEQ ID NO:59 | 69 | 26 | 2275 |
| Reverse | 5′-AGTGTGATGTAGTGGGGACTTG-3′ SEQ ID NO:60 | 59 | 22 | 2302 |

TABLE 20
Panel 1.2

| | Relative | | Relative |
|---|---|---|---|
| | Expression (%) | | Expression (%) |
| | 1.2tm1539f_ | | 1.2tm1539f_ |
| Tissue Name | ag1322 | Tissue Name | ag1322 |
| Endothelial cells | 0.0 | Renal ca. 786-0 | 0.0 |
| Fetal heart | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal Gland (new | 0.2 | Renal ca. UO-31 | 0.0 |
| lot*). | | | |
| Thyroid | 0.0 | Renal ca. TK-10 | 0.0 |
| Salavary gland | 0.0 | Liver | 0.0 |
| Pituitary gland | 0.0 | Liver (fetal) | 0.2 |
| Brain (fetal) | 0.0 | Liver ca. (hepatoblast) | 0.0 |
| | | HepG2 | |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) | 0.0 |
| | | LX-1 | |
| Brain (hippocampus) | 0.0 | Lung ca. (small cell) | 0.0 |
| | | NCI-H69 | |
| Brain (thalamus) | 0.0 | Lung ca. (s.cell var.) | 0.0 |
| | | SHP-77 | |
| Cerebral Cortex | 0.0 | Lung ca. (large | 0.0 |
| | | cell) NCI-H460 | |
| Spinal cord | 0.0 | Lung ca. (non-sm. cell) | 0.0 |
| | | A549 | |
| CNS ca. (glio/astro) | 0.0 | Lung ca. (non-s.cell) | 0.0 |
| U87-MG | | NCI-H23 | |
| CNS ca. (glio/astro) U- | 0.0 | Lung ca (non-s.cell) | 0.0 |
| 118-MG | | HOP-62 | |
| CNS ca. (astro) SW1783 | 0.0 | Lung ca. (non-s.cl) | 0.0 |
| | | NCI-H522 | |
| CNS ca.* (neuro; met ) | 0.0 | Lung ca. (squam.) SW | 0.0 |
| SK-N-AS | | 900 | |
| CNS ca. (astro) SF-539 | 0.0 | Lung ca. (squam.) NCI- | 0.0 |
| | | H596 | |
| CNS ca. (astro) SNB-75 | 0.0 | Mammary gland | 0.0 |
| CNS ca. (glio) SNB-19 | 0.0 | Breast ca.* (pl. | 0.0 |
| | | effusion) MCF-7 | |
| CNS ca. (glio) U251 | 0.0 | Breast ca.* (pl.ef) | 0.0 |
| | | MDA-MB-231 | |
| CNS ca. (glio) SF-295 | 0.0 | Breast ca.* (pl. | 0.0 |
| | | effusion) T47D | |
| Heart | 0.0 | Breast ca. BT-549 | 0.0 |
| Skeletal Muscle (new | 0.0 | Breast ca. MDA-N | 0.0 |
| lot*) | | | |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 0.0 |
| Colorectal | 0.0 | Ovarian ca. OVCAR-8 | 0.0 |
| Stomach | 0.0 | Ovarian ca. IGROV-1 | 0.0 |
| Small intestine | 0.0 | Ovarian ca.* (ascites) | 0.0 |
| | | SK-OV-3 | |
| Colon ca. SW480 | 0.0 | Uterus | 0.0 |
| Colon ca.* (SW480 | 0.0 | Placenta | 0.0 |
| met) SW620 | | | |
| Colon ca. HT29 | 0.0 | Prostate | 2.1 |
| Colon ca. HCT-116 | 0.0 | Prostate ca.* (bone | 0.0 |
| | | met) PC-3 | |
| Colon ca. CaCo-2 | 0.0 | Testis | 100.0 |
| 83219 CC Well to Mod | 0.0 | Melanoma Hs688 (A) .T | 0.0 |
| Diff (ODO3866) | | | |
| Colon ca. HCC-2998 | 0.0 | Melanoma* (met) | 0.0 |
| | | Hs688 (B) .T | |
| Gastric ca.* (liver | 0.0 | Melanoma UACC-62 | 0.0 |
| met) NCI-N87 | | | |
| Bladder | 0.1 | Melanoma M14 | 0.0 |
| Trachea | 0.0 | Melanoma LOX IMVI | 0.0 |
| Kidney | 0.0 | Melanoma* (met) SK-MEL- | 0.0 |
| | | 5 | |
| Kidney (fetal) | 0.0 | Adipose | 0.0 |

TABLE 21
Panel 1.3D

| | Relative | Relative |
|---|---|---|
| | Expression (%) | Expression (%) |
| | 1.3dtm3287f | 1.3dx4tm535 |
| Tissue Name | _ag2071 | 2f_ag2098_a1 |
| Liver adenocarcinoma | 0.0 | 0.0 |
| Pancreas | 0.0 | 0.0 |
| Pancreatic ca. CAPAN 2 | 0.0 | 0.0 |
| Adrenal gland | 0.0 | 0.0 |
| Thyroid | 0.0 | 0.0 |
| Salivary gland | 0.0 | 0.0 |
| Pituitary gland | 0.0 | 0.0 |
| Brain (fetal) | 0.0 | 0.0 |
| Brain (whole) | 0.0 | 0.0 |
| Brain (amygdala) | 0.0 | 0.0 |
| Brain (cerebellum) | 0.0 | 0.0 |
| Brain (hippocampus) | 0.0 | 0.0 |
| Brain (substantia nigra) | 0.0 | 0.0 |
| Brain (thalamus) | 0.0 | 0.0 |
| Cerebral Cortex | 0.0 | 0.0 |
| Spinal cord | 0.0 | 0.0 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met ) SK-N-AS | 0.0 | 0.0 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.0 | 0.0 |
| CNS ca. (glio) SF-295 | 0.0 | 0.0 |
| Heart (fetal) | 0.0 | 0.0 |
| Heart | 0.0 | 0.0 |
| Fetal Skeletal | 0.0 | 0.0 |
| Skeletal muscle | 0.0 | 0.0 |
| Bone marrow | 0.0 | 0.0 |
| Thymus | 0.0 | 2.0 |
| Spleen | 0.0 | 0.0 |
| Lymph node | 0.0 | 0.0 |
| Colorectal | 0.0 | 0.0 |
| Stomach | 0.0 | 0.0 |
| Small intestine | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| 83219 CC Well to Mod Diff | 0.0 | 0.0 |
| (ODO3866) | | |
| Colon ca. HCC-2998 | 0.0 | 0.0 |
| Gastric ca.* (liver met) NCI-N87 | 0.0 | 0.0 |
| Bladder | 0.0 | 0.0 |
| Trachea | 0.0 | 0.0 |
| Kidney | 0.0 | 0.0 |
| Kidney (fetal) | 0.0 | 0.0 |
| Renal ca. 786-0 | 0.0 | 0.0 |
| Renal ca. A498 | 0.6 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s.cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s.cell) NCI-H23 | 0.0 | 0.0 |
| Lung ca. (non-s.cell) HOP-62 | 0.0 | 0.0 |
| Lung ca. (non-s.cl) NCI-H522 | 0.0 | 0.0 |
| Lung ca. (squam.) SW 900 | 0.0 | 0.0 |
| Lung ca. (squam.) NCI-H596 | 0.0 | 0.0 |
| Mammary gland | 0.0 | 0.0 |
| Breast ca.* (pl. effusion) MCF-7 | 0.0 | 0.0 |
| Breast ca.* (pl.ef) MDA-MB-231 | 0.0 | 0.0 |
| Breast ca.* (pl. effusion) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.0 |
| Uterus | 0.0 | 0.0 |
| Placenta | 0.0 | 0.0 |
| Prostate | 1.6 | 0.0 |
| Prostate ca.* (bone met) PC-3 | 0.0 | 0.0 |
| Testis | 100.0 | 100.0 |
| Melanoma Hs688 (A) .T | 0.0 | 0.0 |
| Melanoma* (met) Hs688 (B) .T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 0.0 | 0.0 |

Panel 1.2 Summary: Agz1322 Expression of the SC30236456_EXT1 gene is highest in testis (CT value=29). However, much lower expression is also seen in prostate (CT value=34.6). Both of these tissues are specific to the male reproductive tract and as such the expression of the SC30236456_EXT1 gene can be used as a marker for these tissues. In addition, the therapeutic modulation of the expression of this gene might be of use in diseases specific to these tissues, including fertility. The SC30236456_EXT1 gene encodes a protein with homology to ADAM proteins, which are membrane disintegrin-metalloproteases. The expression of several other ADAM proteins has been also been shown to be testis-specific and these proteins are thought to play a role in fertilization (ref 1).

Panel 1.3D Summary: Ag2017/Ag2098 Two replicate experiments performed using the same probe/primer set gave similar results to what was observed in Panel 1.2, except in one case in which expression in prostate was not detected. Therefore, expression of the SC30236456_EXT1 gene appears to be restricted to testis and to a lesser extent prostate.

Panel 4D Summary: Ag1322b/Ag2071/A22098. Expression of this gene is low to undetectable (CT values>35) in all of the samples on this panel and thus the data is not shown.

Two novel membrane disintegrin-metalloproteases, ADAM 20 and ADAM 21 were cloned from a human testis cDNA library. Their predicted translation products share 50% sequence identity with each other. Among previously characterized ADAMs, the best similarity was to sperm cell-specific fertilins-alpha and -beta, and meltrin-gamma (ADAM 9) which is ubiquitously expressed. Both ADAM 20 and 21 mRNAs are exclusively expressed in testis, presumably, in analogy to all other testis-specific ADAMs, on mature spermatocytes. Both cDNAs were mapped on the genome, and found to be tightly linked to the same marker (SHGC-36001) on chromosome 14q24.1. This region is not syntenic with the loci of mouse sperm-specific ADAMs 1-5. ADAM 20, but not 21, encodes a consensus Zn2+ binding site of active adamalysin metzincin metalloproteases, and both 20 and 21 encode putative cell-fusion peptides, required for sperm-egg fusion. Based on these characteristics it is possible that ADAM 20 and/or 21 is the functional equivalent of sperm fertilin-alpha, as it was recently reported that this gene is non-functional in humans. See generally, Hooft van Huijsduijnen R. (1998) ADAM 20 and 21; two novel human testis-specific membrane metalloproteases with similarity to fertilin-alpha. Gene 206: 273-282.

### EXAMPLE 5

### Quantitative expression analysis of NOV5a expression in various cells and tissues

Expression of gene SC_{—}86058175_A was assessed using the primer-probe sets Ag1358 and Ag1396 (identical sequences), described in Table DA.

TABLE 22

Probe Name Ag1358/Ag1396

| | | | | |
|---|---|---|---|---|
| | | | | Start |
| Primers | Sequences | TM | Length | Position |

| | | | | |
|---|---|---|---|---|
| Forward | 5′-TTTGATGGGTACCCTCATATGA-3′ | 59.2 | 22 | 63 |
| | SEQ ID NO:61 | | | |
| Probe | TET-5′-TCCAATTAGCCAAGATGAACCTCATGG-3′-TAMRA | 68.8 | 27 | 94 |
| | SEQ ID NO:62 | | | |
| Reverse | 5′-AGGGAGAAGATCTTGGTGATGT-3′ | 59.1 | 22 | 121 |
| | SEQ ID NO:63 | | | |

Expression of this gene in panels 1.2, 1.3D, 2D, 3D and 4D was low/undetectable (CT values > 35) in all samples.

### EXAMPLE 6

### Quantitative expression analysis of NOV6 expression in various cells and tissues

Expression of gene SC_{—}124881299_A was assessed using the primer-probe sets Ag2940 and Ag610, described in Tables EA and EB. Results of the RTQ-PCR runs are shown in Tables EC, ED, EE, and EF.

TABLE 23

Probe Name Ag2940

| | | | | |
|---|---|---|---|---|
| | | | | Start |
| Primers | Sequences | TM | Length | Position |

| | | | | |
|---|---|---|---|---|
| Forward | 5′-CTGAGCTGCATGAAGTATCTGA-3′ | 58.3 | 22 | 15 |
| | SEQ ID NO:64 | | | |
| Probe | TET-5′-TCAATTTCTTCATATTTCTGGGCGGG-3′-TAMRA | 68.5 | 26 | 46 |
| | SEQ ID NO:65 | | | |
| Reverse | 5′-GTCCACCATGACCCAGATG-3′ | 59.7 | 19 | 110 |
| | SEQ ID NO:66 | | | |

TABLE 24

Probe Name Ag610

| | | | | Start |
|---|---|---|---|---|
| Primers | Sequences | TM | Length | Position |
| Forward | 5′-GCACTACCAGGGCAATAACGA-3′ | | 21 | 373 |
| | SEQ ID NO:67 | | | |
| Probe | FAM-5′-ACGTCTTCTCTGCCACCTGGAACTCG-3′-TAMRA | | 26 | 399 |
| | SEQ ID NO:68 | | | |
| Reverse | 5′-GCAGCAACCAAATGTGATCATG-3′ | | 22 | 427 |
| | SEQ ID NO:69 | | | |

TABLE 25
Panel 1.1

| | Relative | | Relative |
|---|---|---|---|
| | Expression (%) | | Expression (%) |
| | 1.1tm767f_{—} | | 1.1tm767f_{—} |
| Tissue Name | ag610 | Tissue Name | ag610 |
| Adipose | 1.8 | Renal ca. TK-10 | 12.0 |
| Adrenal gland | 30.6 | Renal ca. UO-31 | 8.0 |
| Bladder | 5.5 | Renal ca. RXF 393 | 5.1 |
| Brain (amygdala) | 1.7 | Liver | 8.5 |
| Brain (cerebellum) | 85.3 | Liver (fetal) | 3.7 |
| Brain (hippocampus) | 8.2 | Liver ca. | 0.0 |
| | | (hepatoblast) HepG2 | |
| Brain (substantia | 7.5 | Lung | 9.2 |
| nigra) | | | |
| Brain (thalamus) | 5.7 | Lung (fetal) | 13.0 |
| Cerebral Cortex | 2.6 | Lung ca (non-s.cell) | 15.3 |
| | | HOP-62 | |
| Brain (fetal) | 23.8 | Lung ca. (large | 0.0 |
| | | cell) NCI-H460 | |
| Brain (whole) | 6.9 | Lung ca. (non-s.cell) | 1.3 |
| | | NCI-H23 | |
| CNS ca. (glio/astro) | 0.0 | Lung ca. (non-s.cl) | 4.6 |
| U-118-MG | | NCI-H522 | |
| CNS ca. (astro) SF-539 | 0.7 | Lung ca. (non-sm. | 0.3 |
| | | cell) A549 | |
| CNS ca. (astro) SNB-75 | 1.2 | Lung ca. (s.cell var.) | 0.0 |
| | | SHP-77 | |
| CNS ca. (astro) SW1783 | 2.3 | Lung ca. (small cell) | 0.0 |
| | | LX-1 | |
| CNS ca. (glio) U251 | 0.0 | Lung ca. (small cell) | 0.4 |
| | | NCI-H69 | |
| CNS ca. (glio) SF-295 | 9.0 | Lung ca. (squam.) SW | 0.2 |
| | | 900 | |
| CNS ca. (glio) SNB-19 | 0.0 | Lung ca. (squam.) NCI- | 0.6 |
| | | H596 | |
| CNS ca. (glio/astro) | 0.0 | Lymph node | 4.6 |
| U87-MG | | | |
| CNS ca.* (neuro; met.) | 49.7 | Spleen | 3.3 |
| SK-N-AS | | | |
| Mammary gland | 9.7 | Thymus | 1.0 |
| Breast ca. BT-549 | 0.0 | Ovary | 12.1 |
| Breast ca. MDA-N | 0.0 | Ovarian ca. IGROV-1 | 1.6 |
| Breast ca.* (pl. | 0.0 | Ovarian ca. OVCAR-3 | 4.9 |
| effusion) T47D | | | |
| Breast ca.* (pl. | 0.0 | Ovarian ca. OVCAR-4 | 0.5 |
| effusion) MCF-7 | | | |
| Breast ca.* (pl.ef) | 0.0 | Ovarian ca. OVCAR-5 | 2.5 |
| MDA-MB-231 | | | |
| Small intestine | 17.6 | Ovarian ca. OVCAR-8 | 0.0 |
| Colorectal | 4.0 | Ovarian ca.* (ascites) | 8.8 |
| | | SK-OV-3 | |
| Colon ca. HT29 | 0.0 | Pancreas | 8.3 |
| Colon ca. CaCo-2 | 5.4 | Pancreatic ca. CAPAN 2 | 9.7 |
| Colon ca. HCT-15 | 0.0 | Pituitary gland | 6.5 |
| Colon ca. HCT-116 | 4.7 | Placenta | 15.8 |
| Colon ca. HCC-2998 | 0.0 | Prostate | 4.8 |
| Colon ca. SW480 | 0.0 | Prostate ca.* (bone | 0.0 |
| | | met) PC-3 | |
| Colon ca.* (SW480 | 0.0 | Salavary gland | 4.1 |
| met) SW620 | | | |
| Stomach | 9.9 | Trachea | 2.9 |
| Gastric ca.* (liver | 0.0 | Spinal cord | 7.2 |
| met) NCI-N57 | | | |
| Heart | 100.0 | Testis | 4.1 |
| Fetal Skeletal | 27.4 | Thyroid | 10.1 |
| Skeletal muscle | 16.6 | Uterus | 11.1 |
| Endothelial cells | 84.7 | Melanoma M14 | 0.0 |
| Heart (fetal) | 55.1 | Melanoma LOX IMVI | 0.0 |
| Kidney | 43.8 | Melanoma UACC-62 | 0.0 |
| Kidney (fetal) | 12.3 | Melanoma SK-MEL-28 | 0.0 |
| Renal ca. 786-0 | 0.0 | Melanoma* (met) SK- | 2.0 |
| | | MEL-5 | |
| Renal ca. A498 | 0.0 | Melanoma Hs688 (A) .T | 10.1 |
| Renal ca. ACHN | 2.2 | Melanoma* (met) | 3.7 |
| | | Hs688 (B) .T | |
| Liver adenocarcinoma | 0.5 | Kidney (fetal) | 5.6 |
| Pancreas | 2.2 | Renal ca. 786-0 | 0.0 |
| Pancreatic ca. CAPAN 2 | 44.6 | Renal ca. A498 | 10.9 |
| Adrenal gland | 44.6 | Renal ca. RXF 393 | 35.9 |
| Thyroid | 15.3 | Renal ca. ACHN | 1.9 |
| Salivary gland | 1.1 | Renal ca. UO-31 | 2.9 |
| Pituitary gland | 5.9 | Renal ca. TK-10 | 2.7 |
| Brain (fetal) | 38.5 | Liver | 3.8 |
| Brain (whole) | 29.4 | Liver (fetal) | 13.6 |
| Brain (amygdala) | 9.9 | Liver ca. (hepatoblast) | 0.0 |
| | | HepG2 | |
| Brain (cerebellum) | 100.0 | Lung | 24.5 |
| Brain (hippocampus) | 31.7 | Lung (fetal) | 18.2 |
| Brain (substantia | 3.8 | Lung ca. (small cell) | 0.0 |
| nigra) | | LX-1 | |
| Brain (thalamus) | 29.2 | Lung ca. (small cell) | 0.0 |
| | | NCI-H69 | |
| Cerebral Cortex | 2.4 | Lung ca. (s.cell var.) | 0.0 |
| | | SHP-77 | |
| Spinal cord | 18.8 | Lung ca. (large | 0.5 |
| | | cell) NCI-H460 | |
| CNS ca. (glio/astro) | 0.0 | Lung ca. (non-sm. cell) | 0.2 |
| U87-MG | | A549 | |
| CNS ca. (glio/astro) U- | 0.0 | Lung ca. (non-s.cell) | 4.2 |
| 118-MG | | NCI-H23 | |
| CNS ca. (astro) SW1783 | 9.8 | Lung ca (non-s-cell) | 3.2 |
| | | HOP-62 | |
| CNS ca.* (neuro; met | 43.8 | Lung ca. (non-s.cl) | 0.0 |
| SK-N-As | | NCI-H522 | |
| CNS ca. (astro) SF-539 | 3.2 | Lung ca. (squam.) SW | 0.0 |
| | | 900 | |
| CNS ca. (astro) SNB-75 | 17.5 | Lung ca. (squam.) NCI- | 0.2 |
| | | H596 | |
| CNS ca. (glio) SNB-19 | 0.4 | Mammary gland | 15.9 |
| CNS ca. (glio) U251 | 0.3 | Breast ca.* (pl. | 0.0 |
| | | effusion) MCF-7 | |
| CNS ca. (glio) SF-295 | 8.1 | Breast ca.* (pl.ef) | 0.0 |
| | | MDA-MB-231 | |
| Heart (fetal) | 58.6 | Breast ca.* (pl. | 0.0 |
| | | effusion) T47D | |
| Heart | 61.4 | Breast ca. BT-549 | 0.0 |
| Fetal Skeletal | 32.1 | Breast ca. MDA-N | 0.0 |
| Skeletal muscle | 18.1 | Ovary | 12.9 |
| Bone marrow | 1.6 | Ovarian ca. OVCAR-3 | 2.0 |
| Thymus | 3.5 | Ovarian ca. OVCAR-4 | 1.0 |
| Spleen | 13.5 | Ovarian ca. OVCAR-5 | 1.2 |
| Lymph node | 20.9 | Ovarian ca. OVCAR-8 | 0.0 |
| Colorectal | 7.0 | Ovarian ca. IGROV-1 | 0.6 |
| Stomach | 17.8 | Ovarian ca.* (ascites) | 9.1 |
| | | SK-OV-3 | |
| Small intestine | 59.9 | Uterus | 75.2 |
| Colon ca. SW480 | 0.0 | Placenta | 18.5 |
| Colon ca.* (SW480 | 0.0 | Prostate | 10.6 |
| met) SW620 | | | |
| Colon ca. HT29 | 0.0 | Prostate ca.* (bone | 0.0 |
| | | met) PC-3 | |
| Colon ca. HCT-116 | 4.7 | Testis | 10.8 |
| Colon ca. CaCo-2 | 3.3 | Melanoma Hs688 (A) .T | 9.2 |
| 83219 CC Well to Mod | 8.0 | Melanoma* (met) | 1.7 |
| Diff (ODO3866) | | Hs688 (B) .T | |
| Colon ca. HCC-2998 | 0.0 | Melanoma UACC-62 | 0.0 |
| Gastric ca.* (liver | 0.7 | Melanoma M14 | 0.0 |
| met) NCI-N87 | | | |
| Bladder | 1.5 | Melanoma LOX IMVI | 0.0 |
| Trachea | 6.8 | Melanoma* (met) SK-MEL- | 3.7 |
| | | 5 | |
| Kidney | 15.4 | Adipose | 7.8 |

TABLE 27
Panel 2D

| | Relative | | Relative |
|---|---|---|---|
| | Expression (%) | | Expression (%) |
| | 2dtm5571f_{—} | | 2dtm5571f_{—} |
| Tissue Name | ag610 | Tissue Name | ag610 |
| Normal Colon GENPAK | 59.5 | Kidney NAT Clontech | 33.4 |
| 061003 | | 8120608 | |
| 83219 CC Well to Mod | 10.2 | Kidney Cancer Clontech | 14.2 |
| Diff (ODO3866) | | 8120613 | |
| 83220 CC NAT | 15.2 | Kidney NAT Clontech | 70.7 |
| (ODO3866) | | 8120614 | |
| 83221 CC Gr.2 | 5.0 | Kidney Cancer Clontech | 27.9 |
| rectosigmoid | | 9010320 | |
| (ODO3868) | | | |
| 83222 CC NAT | 21.8 | Kidney NAT Clontech | 76.8 |
| (ODO3868) | | 9010321 | |
| 83235 CC Mod Diff | 14.3 | Normal Uterus GENPAK | 14.7 |
| (ODO3920) | | 061018 | |
| 83236 CC NAT | 21.0 | Uterus Cancer GENPAK | 33.2 |
| (ODO3920) | | 064011 | |
| 83237 CC Gr.2 ascend | 9.8 | Normal Thyroid | 19.2 |
| colon (ODO3921) | | Clontech A + 6570-1 | |
| 83238 CC NAT | 12.9 | Thyroid Cancer GENPAK | 3.2 |
| (ODO3921) | | 064010 | |
| 83241 CC from | | | |
| Partial Hepatectomy | 12.3 | Thyroid Cancer | 3.5 |
| (ODO4309) | | INVITROGEN A302152 | |
| 83242 Liver NAT | 7.5 | Thyroid NAT INVITROGEN | 6.9 |
| (ODO4309) | | A302153 | |
| 87472 Colon mets to | 3.2 | Normal Breast GENPAK | 12.7 |
| lung (ODO4451-01) | | 061019 | |
| 87473 Lung NAT | 7.6 | 84877 Breast Cancer | 6.7 |
| (ODO4451-02) | | (ODO4566) | |
| Normal Prostate | 26.8 | 85975 Breast Cancer | 12.0 |
| Clontech A + 6546-1 | | (ODO4590-01) | |
| 84140 Prostate | 12.5 | 85976 Breast Cancer | 26.8 |
| Cancer (ODO4410) | | Mets (ODO4590-03) | |
| 84141 Prostate NAT | 28.9 | 87070 Breast Cancer | 7.1 |
| (OD04410) | | Metastasis (ODO4655- | |
| | | 05) | |
| 87073 Prostate | 11.3 | GENPACK Breast Cancer | 4.2 |
| Cancer (ODO4720-01) | | 064006 | |
| 87074 Prostate NAT | 36.6 | Breast Cancer Res. | 8.4 |
| (ODO4720-02) | | Gen. 1024 | |
| Normal Lung GENPACK | 37.6 | Breast Cancer Clontech | 12.1 |
| 061010 | | 9100266 | |
| 83239 Lung Met to | 5.1 | Breast NAT Clontech | 9.9 |
| Muscle (ODO4286) | | 9100265 | |
| 83240 Muscle NAT | 8.1 | Breast Cancer | 19.1 |
| (ODO4286) | | INVITROGEN A209073 | |
| 84136 Lung Malignant | 9.8 | Breast NAT INVITROGEN | 6.3 |
| Cancer (ODO3126) | | A2090734 | |
| 84137 Lung NAT | 43.5 | Normal Liver GENPAK | 1.7 |
| (ODO3126) | | 061009 | |
| 84871 Lung Cancer | 44.4 | Liver Cancer GENPAK | 4.1 |
| (ODO4404) | | 064003 | |
| 84872 Lung NAT | 13.7 | Liver Cancer Research | 4.3 |
| (ODO4404) | | Genetics RNA 1025 | |
| 84875 Lung Cancer | 7.1 | Liver Cancer Research | 10.4 |
| (ODO4565) | | Genetics RNA 1026 | |
| 84876 Lung NAT | 9.4 | Paired Liver Cancer | 5.1 |
| (ODO4565) | | Tissue Research | |
| | | Genetics RNA 6004-T | |
| 85950 Lung Cancer | 5.3 | Paired Liver Tissue | 1.6 |
| (ODO4237-01) | | Research Genetics RNA | |
| | | 6004-N | |
| 85970 Lung NAT | 36.9 | Paired Liver Cancer | 9.1 |
| (ODO4237-02) | | Tissue Research | |
| | | Genetics RNA 6005-T | |
| 83255 Ocular Mel Met | 0.6 | Paired Liver Tissue | 3.7 |
| to Liver (ODO4310) | | Research Genetics RNA | |
| | | 6005-N | |
| 83256 Liver NAT | 6.1 | Normal Bladder GENPAK | 3.2 |
| (ODO4310) | | 061001 | |
| 84139 Melanoma Mets | 0.5 | Bladder Cancer | 3.6 |
| to Lung (ODO4321) | | Research Genetics RNA | |
| | | 1023 | |
| 84138 Lung NAT | 51.4 | Bladder Cancer | 2.9 |
| (ODO4321) | | INVITROGEN A302173 | |
| Normal Kidney GENPAK | 29.1 | 87071 Bladder Cancer | 5.5 |
| 061008 | | (ODO4718-01) | |
| 83786 Kidney Ca, | 41.8 | 87072 Bladder Normal | 46.0 |
| Nuclear grade 2 | | Adjacent (ODO4718- | |
| (ODO4338) | | 03) | |
| 83787 Kidney NAT | 29.5 | Normal Ovary Res. Gen. | 13.5 |
| (ODO4338) | | | |
| 83788 Kidney Ca | 7.1 | Ovarian Cancer GENPAK | 20.7 |
| Nuclear grade ½ | | 064008 | |
| (ODO4339) | | | |
| 83789 Kidney NAT | 46.7 | 87492 Ovary Cancer | 2.3 |
| (ODO4339) | | (ODO4768-07) | |
| 83790 Kidney Ca, | 100.0 | 87493 Ovary NAT | 18.3 |
| Clear cell type | | (ODO4768-08) | |
| (ODO4340) | | | |
| 83791 Kidney NAT | 22.7 | Normal Stomach GENPAK | 47.0 |
| (ODO4340) | | 061017 | |
| 83792 Kidney Ca, | 4.6 | Gastric Cancer | 28.3 |
| Nuclear grade 3 | | Clontech 9060358 | |
| (ODO4348) | | | |
| 83793 Kidney NAT | 11.8 | NAT Stomach Clontech | 10.5 |
| (ODO4348) | | 9060359 | |
| 87474 Kidney Cancer | 26.6 | Gastric Cancer | 43.5 |
| (ODO4622-01) | | Clontech 9060395 | |
| 87475 Kidney NAT | 5.7 | NAT Stomach Clontech | 16.4 |
| (ODO4622-03) | | 9060394 | |
| 85973 Kidney Cancer | 32.8 | Gastric Cancer | 12.7 |
| (ODO4450-01) | | Clontech 9060397 | |
| 85974 Kidney NAT | 12.9 | NAT Stomach Clontech | 4.5 |
| (ODO4450-03) | | 9060396 | |
| Kidney Cancer Clontech | 17.4 | Gastric Cancer GENPAK | 16.7 |
| 8120607 | | 064005 | |

TABLE 28
Panel 4D

| | Relative | Relative |
|---|---|---|
| | Expression (%) | Expression (%) |
| | 4dtm5412t_{—} | 4dxtm5136f_{—} |
| Tissue Name | ag2940 | ag610_b1 |
| 93768_Secondary Th1_anti-CD28/anti-CD3 | 0.7 | 0.5 |
| 93769_Secondary Th2_anti-CD28/anti-CD3 | 0.5 | 0.5 |
| 93770_Secondary Tr1_anti-CD28/anti-CD3 | 0.4 | 0.4 |
| 93573_Secondary Th1_resting day 4-6 in IL-2 | 7.1 | 8.3 |
| 93572_Secondary Th2_resting day 4-6 in IL-2 | 9.7 | 6.8 |
| 93571_Secondary Tr1_resting day 4-6 in IL-2 | 14.0 | 9.1 |
| 93568_primary Th1_anti-CD28/anti-CD3 | 0.9 | 0.3 |
| 93569_primary Th2_anti-CD28/anti-CD3 | 0.7 | 0.6 |
| 93570_primary Tr1_anti-CD28/anti-CD3 | 0.5 | 0.5 |
| 93565_primary Th1_resting dy 4-6 in IL-2 | 39.5 | 52.7 |
| 93566_primary Th2_resting dy 4-6 in IL-2 | 12.6 | 15.7 |
| 93567_primary Tr1_resting dy 4-6 in IL-2 | 13.7 | 15.6 |
| 93351_CD45RA CD4 lymphocyte_anti-CD28/anti-CD3 | 0.5 | 0.6 |
| 93352_CD45RO CD4 lymphocyte_anti-CD28/anti-CD3 | 0.9 | 1.6 |
| 93251_CD8 Lymphocytes_anti-CD28/anti-CD3 | 0.7 | 0.2 |
| 93353_chronic CD8 Lymphocytes 2ry_resting dy 4- | 0.7 | 1.9 |
| 6 in IL-2 | | |
| 93574_chronic CD8 Lymphocytes 2ry_activated | 0.8 | 0.4 |
| CD3/CD28 | | |
| 93354_CD4_none | 10.4 | 9.4 |
| 93252_Secondary Th1/Th2/Tr1_anti-CD95 CH11 | 6.4 | 13.7 |
| 93103_LAK cells_resting | 1.0 | 2.0 |
| 93788_LAK cells_IL-2 | 0.8 | 0.7 |
| 93787_LAK cells_IL-2 + IL-12 | 0.5 | 1.4 |
| 93789_LAK cells_IL-2 + IFN gamma | 1.7 | 2.5 |
| 93790_LAK cells_IL-2 + IL-18 | 2.2 | 1.4 |
| 93104_LAK cells_PMA/ionomycin and IL-18 | 0.4 | 0.5 |
| 93578_NK cells IL-2_resting | 1.4 | 0.6 |
| 93109_Mixed Lymphocyte Reaction_Two Way MLR | 1.7 | 1.2 |
| 93110_Mixed Lymphocyte Reaction_Two Way MLR | 1.4 | 0.7 |
| 93111_Mixed Lymphocyte Reaction_Two Way MLR | 1.0 | 0.2 |
| 93112_Mononuclear Cells (PBMCs)_resting | 7.7 | 6.0 |
| 93113_Mononuclear Cells (PBMCs)_PWM | 1.9 | 2.3 |
| 93114_Mononuclear Cells (PBMCs)_PHA-L | 1.7 | 3.6 |
| 93249_Ramos (B cell)_none | 0.0 | 0.0 |
| 93250_Ramos (B cell)_ionomycin | 0.0 | 0.0 |
| 93349_B lymphocytes_PWM | 0.9 | 2.1 |
| 93350_B lymphoytes_CD40L and IL-4 | 1.1 | 0.7 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.3 |
| 93776 Monocytes_LPS 50 ng/ml | 0.0 | 0.0 |
| 93581 Macrophages_resting | 0.1 | 0.4 |
| 93582 Macrophages_LPS 100 ng/ml | 0.0 | 0.0 |
| 93098_HUVEC (Endothelial)_none | 24.8 | 25.0 |
| 93099_HUVEC (Endothelial)_starved | 51.4 | 70.2 |
| 93100_HUVEC (Endothelial)_IL-1b | 21.5 | 24.4 |
| 93779_HUVEC (Endothelial)_IFN gamma | 61.6 | 36.6 |
| 93102_HUVEC (Endothelial)_TNF alpha + IFN | 6.9 | 6.6 |
| gamma | | |
| 93101_HUVEC (Endothelial)_TNF alpha + IL4 | 6.9 | 4.8 |
| 93781_HUVEC (Endothelial)_IL-11 | 35.8 | 32.6 |
| 93583_Lung Microvascular Endothelial Cells_none | 100.0 | 89.1 |
| 93584_Lung Microvascular Endothelial Cells_TNFa | 27.5 | 38.0 |
| (4 ng/ml) and IL1b (1 ng/ml) | | |
| 92662_Microvascular Dermal endothelium_none | 94.6 | 100.0 |
| 92663_Microsvasular Dermal endothelium_TNFa (4 | 32.5 | 45.0 |
| ng/ml) and IL1b (1 ng/ml) | | |
| 93773_Bronchial epithelium_TNFa (4 ng/ml) and | 0.0 | 0.0 |
| IL1b (1 ng/ml)** | | |
| 93347_Small Airway Epithelium_none | 0.0 | 0.0 |
| 93348_Small Airway Epithelium_TNFa (4 ng/ml) | 0.3 | 0.1 |
| and IL1b (1 ng/ml) | | |
| 92668_Coronery Artery SMC_resting | 0.0 | 0.2 |
| 92669_Coronery Artery SMC_TNFa (4 ng/ml) and | 0.0 | 0.0 |
| IL1b (1 ng/ml) | | |
| 93107_astrocytes_resting | 10.5 | 11.6 |
| 93108_astrocytes_TNFa (4 ng/ml) and IL1b (1 | 9.2 | 9.0 |
| ng/ml) | | |
| 92666_KU-812 (Basophil)_resting | 0.2 | 1.9 |
| 92667_KU-812 (Basophil)_PMA/ionoycin | 3.2 | 4.6 |
| 93579_CCD1106 (Keratinocytes)_none | 0.0 | 0.0 |
| 93580_CCD1106 (Keratinocytes)_TNFa and IFNg** | 0.0 | 0.0 |
| 93791_Liver Cirrhosis | 1.1 | 1.4 |
| 93792_Lupus Kidney | 2.2 | 2.9 |
| 93577_NCI-H292 | 1.0 | 1.1 |
| 93358_NCI-H292_IL-4 | 0.9 | 1.9 |
| 93360_NCI-H292_IL-9 | 0.5 | 1.7 |
| 93359_NCI-H292_IL-13 | 1.7 | 1.1 |
| 93357_NCI-H292_IFN gamma | 0.9 | 1.4 |
| 93777_HPAEC_- | 27.2 | 21.4 |
| 93778_HPAEC_IL-1 beta/TNA alpha | 16.8 | 8.9 |
| 93254_Normal Human Lung Fibroblast_none | 0.0 | 0.0 |
| 93253_Normal Human Lung Fibroblast_TNFa (4 | 0.0 | 0.0 |
| ng/ml) and IL-1b (1 ng/ml) | | |
| 93257_Normal Human Lung Fibrobiast_IL-4 | 0.0 | 0.1 |
| 93256_Normal Human Lung Fibrobiast_IL-9 | 0.0 | 0.0 |
| 93255_Normal Human Lung Fibroblast_IL-13 | 0.0 | 0.2 |
| 93258_Normal Human Lung Fibroblast_IFN gamma | 0.0 | 0.0 |
| 93106_Dermal Fibroblasts CCD1070_resting | 1.3 | 2.1 |
| 93361_Dermal Fibrobiasts CCD1070_TNF alpha 4 | 7.3 | 10.5 |
| ng/ml | | |
| 93105_Dermal Fibroblasts CCD1070_IL-1 beta 1 | 0.8 | 0.7 |
| ng/ml | | |
| 93772_dermal fibroblast_IFN gamma | 0.0 | 0.0 |
| 93771_dermal fibroblast_IL-4 | 0.7 | 0.2 |
| 93259_IBD Colitis 1** | 7.3 | 8.1 |
| 93260_IBD Colitis 2 | 2.0 | 1.5 |
| 93261_IBD Crohns | 4.1 | 2.5 |
| 735010_Colon normal | 33.7 | 26.9 |
| 735019_Lung_none | 52.8 | 62.1 |
| 64028-1_Thymus_none | 48.6 | 41.9 |
| 64030-1_Kidney_none | 2.4 | 3.4 |

Panel 1.1 Summary: Ag610 The SC_{—}124881299_A gene is highly expressed in a number of samples on this panel. Highest expression is detected in adult heart (CT value=22.7). This observation suggests that the SC_{—}124881299_A gene may play a role in heart homeostasis. Thus, therapeutic modulation of the expression of this gene might be useful in the treatment of heart diseases, including cardiomyopathy, atherosclerosis, hypertension, congenital heart defects, aortic stenosis, atrial septal defect (asd), atrioventricular (a-v) canal defect, ductus arteriosus, pulmonary stenosis, subaortic stenosis, ventricular septal defect (vsd), and valve diseases, or may aid recovery after damage to the heart. In general, expression of this gene is associated with normal tissues but not with cancer cell lines. Expression of the SC_{—}124881299_A gene is high in many regions of the brain, including the amygdala, thalamus, cerebellum, and cerebral cortex, with highest expression in the cerebellum (CT value=22.9). This observation suggests that the SC_{—}124881299A gene may be involved in normal brain function and that disregulation of its expression may play a role in neurological diseases. This gene is also moderately expressed in adrenal gland, pituitary gland, thyroid, skeletal muscle, liver, and pancreas. Expression in the metabolic tissues skeletal muscle, liver and pancreas suggest that the SC_{—}124881299_A gene may be involved in metabolic control processes and serve as a drug target for metabolic diseases, including obesity and diabetes. In addition, this gene may play a role in normal neuroendocrine function and disregulation may lead to disease. Interestingly, the gene is expressed to very high levels in normal mammary gland (CT value=26) but appears to be absent in 5/5 breast cancer cell lines. The SC_{—}124881299_A gene is also relatively under expressed in several CNS cancer cell lines relative to the normal brain. Therefore, the SC_{—}124881299_A gene product has potential utility as a protein therapeutic in the treatment of breast and CNS cancers. Please note that expression in adipose is skewed by the presence of genomic DNA contamination in this sample.

Panel 1.3D Summary: Ag610 Expression of the SC_{—}124881299_A gene is associated primarily with normal tissue samples rather than the samples derived from the cultured cell lines on this panel, consistent with what was observed on Panel 1.1. Strikingly, this gene appears to be under expressed in ovarian, breast and lung cancer cell lines relative to normal controls. These observations suggest that the SC_{—}124881299_A gene product may have utility as a protein therapeutic in the treatment of ovarian, breast and lung cancers.

The SC_{—}124881299_A gene is also moderately expressed in many regions of the brain, including the amygdala, thalamus, hippocampus, and cerebral cortex, with highest expression in the cerebellum (CT value=28.6). Expression is also detected in the spinal cord. The gene encoded by the SC_{—}124881299_A gene encodes a putative neural tetraspanin. Tetraspanins are involved in neuron to astrocyte signalling (ref 1). Astrocytes are of interest in neuronal regeneration as they form glial scars in response to CNS injury (i.e., spinal cord injury, brain trauma, etc). Glial scars form a physical barrier to growing axons and dendrites, limiting the amout of CNS repair possible. Astrocytes are also critical to the process of compensatory synaptogenesis in that they are integral in the brain's cholesterol transport system and are involved in the transport of hydrophobic membrane/synapse components to neurons. The selective modulations and/or activation of this protein could therefore be of therapeutic value in the treatment of CNS injury (stroke, head trauma, spinal cord injury) or neurodegeneration (Alzheimer's, Parkinson's, Huntington's, spinocerebellar ataxia, etc).

In addition, low expression of this gene is detected in pancreas, liver and adipose with moderate expression in adrenal gland, thyroid, pituitary gland, heart, and skeletal muscle. Expression in the metabolic tissues skeletal muscle, liver and pancreas suggest that the SC_{—}124881299_A gene may be involved in metabolic control processes and serve as a drug target for metabolic diseases, including obesity and diabetes. In addition, this gene may play a role in normal neuroendocrine function and disregulation may lead to diseases of the endocrine system.

Panel 2D Summary: Ag610 The SC_{—}124881299_A gene is widely expressed among the samples in panel 2D with the highest expression occurring in a kidney cancer sample (CT value =28.2). Of specific interest is the differential over expression of the SC_{—}124881299_A gene in 4/9 kidney cancers and 4/4 gastric cancers relative to the adjacent normal tissue controls. In addition, there is also slight under expression in 2/2 prostate cancers and 5/6 colon cancers relative to the adjacent normal tissue controls. Thus, therapeutic modulation of the expression of the SC_{—}124881299_A gene could have beneficial consequences to the treatment of several types of cancers.

Panel 4D Summary: Ag610/AR2940 The SC_{—}124881299_A transcript is expressed in normal organs, untreated endothelial cells and polarized resting T cells. Furthermore, expression is reduced in endothelial cells treated with IL-1 and TNF alpha. The expression pattern is consistent in two experiments using different probe/primer sets. Protein therapeutics designed with the protein encoded for by this transcript could interact with the cognate ligand for the SC_{—}124881299_A protein to reduce or inhibit inflammation due to the exposure of endothelium to the pro-inflammatory cytokines. Alternatively, since many tetraspanins are involved as part of a receptor complexes, the putative tetraspanin encoded by the SC_{—}124881299_A gene may actually function in the initial steps of activation and, therefore, an antibody against the protein encoded for by this transcript may block subsequent steps of endothelial cell activation. Both of these therapeutics may be important in the treatment of diseases such as asthma, emphysema, arthritis, allergy, psoriasis and IBD.

Panel CNSD.01 Summary: Ag610 The SC_{—}124881299_A gene is expressed at low to undetectable levels (CT values>34.5) in all of the samples on this panel; however, the brain tissues in which highest expression was observed in Panels 1.1 and 1.3D are not represented here.

Astrocytes respond to contact with neurons by cell-cycle arrest and complex process formation. In our effort to discover the molecular mechanisms that underlie this phenomenon we have identified a known tetraspanin, CD81, as a critical component of astrocyte responses to neuronal differentiation signals. Here we show that CD81 is expressed on the surface of the astrocyte and that its expression level can be modulated by contact with neurons. Further, using three separate antibodies, 2F7, Eat1, and Eat2, which recognize unique epitopes in the extracellular domains of the CD81 protein, we show that there is a unique domain, recognized by Eat1, that is required for astrocyte cell-cycle withdrawal in response to neurons. This is likely due to conformational changes in the CD81 molecule, as inclusion of 2F7 actually augments neuron-induced astrocyte growth arrest. The critical nature of CD81 in normal astrocyte-neuron biology was confirmed by using mice in which CD81 had been deleted by homologous recombination. Astrocytes null at the CD81 locus were blind to the proliferative arrest encoded on the neuronal cell surface. Taken together, these data strongly suggest that CD81 is a critical regulator of neuron-induced astrocytic differentiation. See generally, Kelic S., Levy S., Suarez C., Weinstein D. E. (2001) CD81 regulates neuron-induced astrocyte cell-cycle exit. Mol. Cell. Neurosci. 17: 551-560.

### EXAMPLE 7

### Quantitative expression analysis of NOV7 expression in various cells and tissues

Expression of gene SC_{—}18468704_A was assessed using the primer-probe sets Ag651 and Ag652, described in Tables FA and FB. Results of the RTQ-PCR runs are shown in Tables FC and FD.

TABLE 29

Probe Name Ag651

| | | | | |
|---|---|---|---|---|
| | | | | Start |
| Primers | Sequences | TM | Length | Position |

| | | | | |
|---|---|---|---|---|
| Forward | 5′-CGGGAAAGGAAGATCAACTC-3′ | 58.7 | 20 | 1079 |
| | SEQ ID NO:70 | | | |
| Probe | FAM-5′-CTCCCAGACCGCGTGCTGAACTT-3′-TAMRA | 70.9 | 23 | 1109 |
| | SEQ ID NO:71 | | | |
| Reverse | 5′-CATCAGGAAGTGGTCCTTGAG-3′ | 59.7 | 21 | 1133 |
| | SEQ ID NO:72 | | | |

TABLE 30

Probe Name Ag652

| | | | | Start |
|---|---|---|---|---|
| Primers | Sequences | TM | Length | Position |
| Forward | 5′-CGCGACTGTCAAAACTACATC-3′ | 58.5 | 21 | 104 |
| | SEQ ID NO:73 | | | |
| Probe | TET-5′-AGTCACCTGTTCACCTGTGGCACAG-3′-TAMRA | 68.9 | 25 | 149 |
| | SEQ ID NO:74 | | | |
| Reverse | 5′-GTAGGTACACATGGGGCTGAA-3′ | 59.9 | 21 | 179 |
| | SEQ ID NO:75 | | | |

TABLE 31
Panel 1.1

| | Relative | Relative |
|---|---|---|
| | Expression (%) | Expression (%) |
| | 1.1tm812f_ | 1.1tm812t_ |
| Tissue Name | ag651 | ag652 |
| Adipose | 1.0 | 0.8 |
| Adrenal gland | 13.9 | 9.5 |
| Bladder | 37.9 | 27.0 |
| Brain (amygdala) | 3.2 | 2.1 |
| Brain (cerebellum) | 14.8 | 12.4 |
| Brain (hippocampus) | 8.5 | 5.9 |
| Brain (substantia nigra) | 20.4 | 17.7 |
| Brain (thalamus) | 5.4 | 4.2 |
| Cerebral Cortex | 15.1 | 13.4 |
| Brain (fetal) | 11.8 | 8.5 |
| Brain (whole) | 10.8 | 7.6 |
| CNS ca. (glio/astro) U-118-MG | 1.7 | 1.7 |
| CNS ca. (astro) SF-539 | 4.6 | 4.4 |
| CNS ca. (astro) SNB-75 | 1.4 | 1.2 |
| CNS ca. (astro) SW1783 | 4.9 | 5.1 |
| CNS ca. (glio) U251 | 14.5 | 14.3 |
| CNS ca. (glio) SF-295 | 7.5 | 9.2 |
| CNS ca. (glio) SNB-19 | 8.8 | 9.5 |
| CNS ca. (glio/astro) U87-MG | 21.5 | 23.5 |
| CNS ca.* (neuro; met ) SK-N-AS | 15.6 | 15.0 |
| Mammary gland | 20.7 | 17.0 |
| Breast ca. BT-549 | 15.3 | 14.9 |
| Breast ca. MDA-N | 2.7 | 2.5 |
| Breast ca.* (pl. effusion) T47D | 16.3 | 16.3 |
| Breast ca.* (pl. effusion) MCF-7 | 17.2 | 17.4 |
| Breast ca.* (pl.ef) MDA-MB-231 | 9.6 | 9.1 |
| Small intestine | 14.8 | 11.9 |
| Colorectal | 12.0 | 8.7 |
| Colon ca. HT29 | 31.6 | 36.1 |
| Colon ca. CaCo-2 | 4.6 | 4.2 |
| Colon ca. HCT-15 | 5.9 | 6.7 |
| Colon ca. HCT-116 | 8.0 | 8.8 |
| Colon ca. HCC-2998 | 21.5 | 20.6 |
| Colon ca. SW480 | 2.3 | 2.1 |
| Colon ca.* (SW480 met) SW620 | 5.5 | 5.0 |
| Stomach | 12.5 | 9.5 |
| Gastric ca.* (liver met) NCI-N87 | 21.5 | 19.5 |
| Heart | 17.0 | 14.4 |
| Fetal Skeletal | 3.2 | 3.5 |
| Skeletal muscle | 10.0 | 8.0 |
| Endothelial Cells | 8.2 | 6.7 |
| Heart (fetal) | 15.6 | 19.8 |
| Kidney | 22.7 | 19.2 |
| Kidney (fetal) | 12.2 | 9.7 |
| Renal ca. 786-0 | 41.8 | 43.2 |
| Renal ca. A498 | 68.3 | 86.5 |
| Renal ca. ACHN | 17.6 | 17.2 |
| Renal ca. TK-10 | 13.7 | 18.3 |
| Renal ca. UO-31 | 10.6 | 11.8 |
| Renal ca. RXF 393 | 18.3 | 16.5 |
| Liver | 7.7 | 4.6 |
| Liver (fetal) | 5.1 | 4.5 |
| Liver ca. (hepatoblast) HepG2 | 5.5 | 6.1 |
| Lung | 9.3 | 6.2 |
| Lung (fetal) | 9.6 | 6.2 |
| Lung ca (non-s.cell) HOP-62 | 100.0 | 100.0 |
| Lung ca. (large cell) NCI-H460 | 14.2 | 15.7 |
| Lung ca. (non-s.cell) NCI-H23 | 15.3 | 15.9 |
| Lung ca. (non-s.cl) NCI-H522 | 10.7 | 12.2 |
| Lung ca. (non-sm. cell) A549 | 28.9 | 30.6 |
| Lung ca. (s.cell var.) SHP-77 | 5.2 | 4.5 |
| Lung ca. (small cell) LX-1 | 20.4 | 18.8 |
| Lung ca. (small cell) NCI-H69 | 1.9 | 2.2 |
| Lung ca. (squam.) SW 900 | 18.9 | 16.8 |
| Lung ca. (squam.) NCI-H596 | 3.7 | 4.2 |
| Lymph node | 6.2 | 4.9 |
| Spleen | 4.6 | 3.5 |
| Thymus | 3.9 | 4.1 |
| Ovary | 17.2 | 14.4 |
| Ovarian ca. IGROV-1 | 39.8 | 42.6 |
| Ovarian ca. OVCAR-3 | 12.4 | 11.3 |
| Ovarian ca. OVCAR-4 | 5.9 | 7.3 |
| Ovarian ca. OVCAR-5 | 66.4 | 70.2 |
| Ovarian ca. OVCAR-8 | 3.4 | 4.5 |
| Ovarian ca.* (ascites) SK-OV-3 | 68.8 | 68.3 |
| Pancreas | 39.2 | 39.2 |
| Pancreatic ca. CAPAN 2 | 16.7 | 17.4 |
| Pituitary gland | 14.8 | 14.1 |
| Placenta | 17.9 | 15.9 |
| Prostate | 12.7 | 10.6 |
| Prostate ca.* (bone met) PC-3 | 71.2 | 62.8 |
| Salavary gland | 26.8 | 22.5 |
| Trachea | 21.0 | 20.3 |
| Spinal cord | 8.4 | 7.6 |
| Testis | 5.3 | 4.7 |
| Thyroid | 25.5 | 24.0 |
| Uterus | 3.0 | 2.1 |
| Melanoma M14 | 5.8 | 5.8 |
| Melanoma LOX IMVI | 10.1 | 9.2 |
| Melanoma UACC-62 | 4.3 | 3.8 |
| Melanoma SK-MEL-28 | 12.3 | 12.2 |
| Melanoma* (met) SK-MEL-5 | 3.8 | 3.1 |
| Melanoma Hs688 (A) .T | 0.5 | 0.4 |
| Melanoma* (met) Hs688 (B) .T | 0.6 | 0.5 |

TABLE 32
Panel 1.2

| | Relative |
|---|---|
| | Expression (%) |
| Tissue Name | 1.2tm1255t_ag652 |
| Endothelial cells | 4.4 |
| Heart (fetal) | 27.0 |
| Pancreas | 5.4 |
| Pancreatic ca. CAPAN 2 | 25.2 |
| Adrenal Gland (new | 26.2 |
| lot*) | |
| Thyroid | 10.6 |
| Salavary gland | 27.5 |
| Pituitary gland | 6.6 |
| Brain (fetal) | 7.9 |
| Brain (whole) | 16.8 |
| Brain (amygdala) | 8.5 |
| Brain (cerebellum) | 13.2 |
| Brain (hippocampus) | 14.6 |
| Brain (thalamus) | 8.3 |
| Cerebral Cortex | 31.2 |
| Spinal cord | 8.8 |
| CNS ca. (glio/astro) | 15.9 |
| U87-MG | |
| CNS ca. (glio/astro) | 0.9 |
| U-118-MG | |
| CNS ca. (astro) SW1783 | 4.1 |
| CNS ca.* (neuro; met) | 6.0 |
| SK-N-AS | |
| CNS ca. (astro) SF-539 | 1.5 |
| CNS ca. (astro) SNB-75 | 1.5 |
| CNS ca. (glio) SNB-19 | 15.2 |
| CNS ca. (glio) U251 | 23.8 |
| CNS ca. (glio) SF-295 | 6.9 |
| Heart | 15.0 |
| Skeletal Muscle (new | 11.4 |
| lot*) | |
| Bone marrow | 5.9 |
| Thymus | 4.1 |
| Spleen | 7.7 |
| Lymph node | 11.2 |
| Colorectal | 7.4 |
| Stomach | 41.8 |
| Small intestine | 11.8 |
| Colon ca. SW480 | 0.6 |
| Colon ca.* (SW480 | 2.2 |
| met) SW620 | |
| Colon ca. HT29 | 21.6 |
| Colon ca. HCT-116 | 7.0 |
| Colon ca. CaCo-2 | 3.1 |
| 83219 CC Well to Mod | 5.4 |
| Diff (ODO3866) | |
| Colon ca. HCC-2998 | 20.6 |
| Gastric ca.* (liver | 17.9 |
| met) NCI-N87 | |
| Bladder | 26.2 |
| Trachea | 100.0 |
| Kidney | 10.9 |
| Kidney (fetal) | 27.7 |
| Renal ca. 786-0 | 50.7 |
| Renal ca. A498 | 56.3 |
| Renal ca. RXF 393 | 26.6 |
| Renal ca. ACHN | 13.4 |
| Renal ca. UO-31 | 6.2 |
| Renal ca. TK-10 | 12.2 |
| Liver | 11.2 |
| Liver (fetal) | 12.9 |
| Liver ca. (hepatoblast) | 7.6 |
| HepG2 | |
| Lung | 28.1 |
| Lung (fetal) | 9.2 |
| Lung ca. (small cell) | 8.0 |
| LX-1 | |
| Lung ca. (small cell) | 1.2 |
| NCI-H69 | |
| Lung ca. (s. cell var.) | 5.4 |
| SHP-77 | |
| Lung ca. (large | 24.7 |
| cell) NCI-H460 | |
| Lung ca. (non-sm. cell) | 31.9 |
| A549 | |
| Lung ca. (non-s. cell) | 23.8 |
| NCI-H23 | |
| Lung ca (non-s. cell) | 16.5 |
| HOP-62 | |
| Lung ca. (non-s. cl) | 7.2 |
| NCI-H522 | |
| Lung ca. (squam.) | 27.2 |
| SW900 | |
| Lung ca. (squam.) | 1.8 |
| NCI-H596 | |
| Mammary gland | 18.3 |
| Breast ca.* (pl. | 22.7 |
| effusion) MCF-7 | |
| Breast ca.* (pl. ef) | 7.5 |
| MDA-MB-231 | |
| Breast ca.* (pl. | 14.2 |
| effusion) T47D | |
| Breast ca. BT-549 | 43.5 |
| Breast ca. MDA-N | 3.2 |
| Ovary | 12.7 |
| Ovarian ca. OVCAR-3 | 4.9 |
| Ovarian ca. OVCAR-4 | 7.4 |
| Ovarian ca. OVCAR-5 | 50.3 |
| Ovarian ca. OVCAR-8 | 6.6 |
| Ovarian ca. IGROV-1 | 23.0 |
| Ovarian ca.* (ascites) | 69.7 |
| SK-OV-3 | |
| Uterus | 8.1 |
| Placenta | 28.7 |
| Prostate | 23.8 |
| Prostate ca.* (bone | 73.7 |
| met) PC-3 | |
| Testis | 8.3 |
| Melanoma Hs688(A).T | 0.0 |
| Melanoma* (met) | 0.2 |
| Hs688(B).T | |
| Melanoma UACC-62 | 4.8 |
| Melanoma M14 | 4.4 |
| Melanoma LOX IMVI | 7.5 |
| Melanoma* (met) SK-MEL-5 | 2.8 |
| Adipose | 10.9 |

Panel 1.1 Summary: Ag651/Ag652 The results from two replicate experiments performed using different probe/primer sets are in excellent agreement. The SC_{—}18468704_A gene appears to be expressed at very high levels in all the samples on this panel. It appears to have its highest expression in one lung cancer cell line (CT value=22) and the gene is over-expressed in a prostate cancer cell line as well as ovarian cancer cell lines, relative to their corresponding normal controls. These observations indicate that the SC_{—}18468704₁₃ A gene may play a role in the development or progression of lung, prostate or ovarian cancer and, as such, therapeutic modulation of this gene might be of utility in the treatment of these diseases. In particular, antibodies raised against the SC_{—}18468704_A protein or small molecules that inhibit the activity of this protein could be beneficial for the treatment of these cancers.

Panel 1.2 Summary: Ag652 The SC_{—}18468704_A gene is expressed widely across the samples of Panel 1.2. The gene appears to be most highly expressed in normal trachea (CT value=23.5). In addition, over-expression of the SC_{—}18468704 A gene is seen in a sample from a prostate cancer cell line as well as in 3/6 ovarian cancer cell lines. These results are consistent with what was seen in Panel 1.1. These observations indicate that the SC18468704A gene may play a role in prostate and ovarian cancer and in the normal tissue homeostasis of the trachea. As such, therapeutic modulation of this gene might be of utility in the treatment of these diseases.

The SC_{—}18468704_A gene is also highly expressed in many regions of the brain, including the amygdala, thalamus, hippocampus, and cerebellum, with highest expression in the cerebral cortex (CT value=25.2). High expression is also detected in the spinal cord. These results are consistent with what was seen in Panel 1.1. The SC_{—}18468704_A gene encodes a putative semaphorin. Semaphorins can act as axon guidance proteins, specifically as chemorepellents that inhibit CNS regenerative capacity. Therefore, manipulation of levels of this protein may be of use in inducing a compensatory synaptogenic response to neuronal death in Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss.

Furthermore, the SC_{—}18468704_A gene is highly expressed (CT values<28) in adrenal gland, skeletal muscle, liver and pancreas. These results are consistent with what was seen in Panel 1.1. The role of semaphorin C in these tissues is not known, but as a transmembrane protein, semaphorin C may be involved in signal transduction pathways. Therefore, the SC_{—}18468704_A gene product may be a drug target for the treatment of diseases involving these tissues, including diabetes, Von Hippel-Lindau (VHL) syndrome, pancreatitis, obesity, adrenoleukodystrophy, congenital adrenal hyperplasia, muscular dystrophy, Lesch-Nyhan syndrome, and myasthenia gravis.

Semaphorins, the plexin family of semaphorin receptors, and scatter factor receptors share evolutionarily conserved protein modules, such as the semaphorin domain and Met Related Sequences (MRS). All these proteins also have in common a role in mediating cell guidance cues. During development, scatter factor receptors control cell migration, epithelial tubulogenesis, and neurite extension. Semaphorins and their receptors are known signals for axon guidance; they are also suspected to regulate developmental processes involving cell migration and morphogenesis, and have been implicated in immune function and tumor progression. Scatter factors and secreted semaphorins are diffusible ligands, whereas membrane-bound semaphorins signal by cell-cell interaction. Cell guidance control by semaphorins requires plexins, alone or in a receptor complex with neuropilins. Semaphorins, besides their role in axon guidance, are expected to have multiple functions in morphogenesis and tissue remodeling by mediating cell-repelling cues through plexin receptors. See generally, Artigiani S., Comoglio P. M., Tamagnone L. (1999) Plexins, semaphorins, and scatter factor receptors: a common root for cell guidance signals? IUBMB Life 48: 477-482.

Progressive axon outgrowth during neural development contrasts with the failure of regenerative neurite growth in the mature mammalian central nervous system (CNS). During neuroembryogenesis, spatiotemporal patterns of repellent and attractant activities in the vicinity of the growth cone favor neurite outgrowth. In the mature CNS, however, a relative balance between forces supporting and restricting axon growth has been established, only allowing subtle morphological changes in existing neuritic arbors and synapses. Following CNS injury, this balance shifts towards enhanced expression of growth-inhibiting molecules and diminished availability of their growth-promoting counterparts. Evidence is now emerging that the proteins governing developmental axon guidance critically contribute to the failure of injured central neurons to regenerate. As a first step toward elucidation of the role of chemorepulsive axon guidance signals in axonal regeneration, the effects of lesions of the central and peripheral nervous system on the expression of Semaphorin3A, the prototype and founding member of the semaphorin family of axon guidance signals, and of the Semaphorin3A receptor proteins neuropilin-1 and plexin-A1 have recently been examined. Here we review the first evidence indicating that (i) lesion-induced changes in the expression of chemorepulsive semaphorins relate to the success or failure of injured neurons to regenerate and (ii) semaphorins may represent important molecular signals controlling multiple aspects of the cellular response that follows CNS injury. In the future, genetic manipulation of the injury-induced changes in the availability of semaphorins and/or of their receptors will provide further insight into the mechanisms by which semaphorins influence neural regeneration. See generally, Pasterkamp R. J., Verhaagen J. (2001) Emerging roles for semaphorins in neural regeneration. Brain Res. Brain Res. Rev. 35: 36-54.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ IDNOS: 2, 4, 6, 8, 10, 12, 14, 16, and 18;
(b) a variant of a mature form of an amino acid sequence selected from the group consisting of SEQ ID NOS :2, 4, 6, 8, 10, 12, 14, 16, and 18, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, and 18; and
(d) a variant of an amino acid sequence selected from the group consisting of SEQ ID NOS: 2, 4, 6, 8, 10, 12, 14, 16, and 18; wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, and 18.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, and 17.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16 and 18;
(b) a variant of a mature form of an amino acid sequence selected from the group consisting of SEQ ID NOS: 2, 4, 6, 8, 10, 12, 14, 16, and 18, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, and 18;
(d) a variant of an amino acid sequence selected from the group consisting SEQ ID NOS: 2, 4, 6, 8, 10, 12, 14, 16, and 18, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, and 18, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, and 17.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, and 17;
(b) a nucleotide sequence differing by one or more nucleotides from a nucleotide sequence selected from the group consisting of SEQ ID NOS: 1, 3, 5, 7, 9, 11, 13, 15, and 17, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, and 17, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26, wherein said subject is a human.

28. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

29. The method of claim 28, wherein said subject is a human.

30. A method of treating or preventing a NOVX-associated disorder, said method comprising administering to a subject in which such treatment or prevention is desired the antibody of claim 15 in an amount sufficient to treat or prevent said NOVX-associated disorder in said subject.

31. The method of claim 30, wherein the subject is a human.

32. A pharmaceutical composition comprising the polypeptide of claim 1 and a pharmaceutically-acceptable carrier.

33. A pharmaceutical composition comprising the nucleic acid molecule of claim 5 and a pharmaceutically-acceptable carrier.

34. A pharmaceutical composition comprising the antibody of claim 15 and a pharmaceutically-acceptable carrier.

35. A kit comprising in one or more containers, the pharmaceutical composition of claim 32.

36. A kit comprising in one or more containers, the pharmaceutical composition of claim 33.

37. A kit comprising in one or more containers, the pharmaceutical composition of claim 34.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

39. The method of claim 38 wherein the predisposition is to cancers.

40. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

41. The method of claim 40 wherein the predisposition is to a cancer.

42. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, and 18, or a biologically active fragment thereof.

43. A method of treating a pathological state in a mammal, the method comprising administering to the mammal the antibody of claim 15 in an amount sufficient to alleviate the pathological state.
